SEQUENCE LISTING (44 sequences)

SEQ ID NO: 1 - DNA, 1380 bp, Arabidopsis thaliana
atggcaccagcacgaacccgagtcaactcactcaacgtggcagtgatcggagccggagccgccggactcgtagctgcaagagagctccgccgcgagaatcacaccgtcgtcgttttcgaacgtgactcaaaagtcggaggtctctgggtatacacacctaacagcgaaccagacccgcttagcctcgatccaaaccgaaccatcgtccattcaagcgtctatgattctctccgaaccaatctcccacgagagtgcatgggttacagagacttccccttcgtgcctcgacctgaagatgacgaatcaagagactcgagaaggtaccctagtcacagagaagttcttgcttaccttgaagacttcgctagagaattcaaacttgtggagatggttcgatttaagaccgaagtagttcttgtcgagcctgaagataagaaatggagggttcaatccaaaaattcagatgggatctccaaagatgagatctttgatgctgttgttgtttgtaatggacattatacagaacctagagttgctcatgttcctggtatagattcatggccagggaagcagattcatagccacaattaccgtgttcctgatcaattcaaagaccaggtggtggtagtgataggaaattttgcgagtggagctgatatcagcagggacataacgggagtggctaaagaagtccatatcgcgtctagatcgaatccatctaagacatactcaaaacttcccgggtcaaacaatctatggcttcactctatgatagaaagtgtacacgaagatgggacgattgtttttcagaacggtaaggttgtacaagctgataccattgtgcattgcactggttacaaatatcacttcccatttctcaacaccaatggctatattactgttgaggataactgtgttggaccgctttacgaacatgtctttccgcctgcgcttgctcccgggctttccttcatcggtttaccctggatgacactgcaattctttatgtttgagctccaaagcaagtgggtggctgcagctttgtctggccgggtcacacttccttcagaagagaaaatgatggaagacgttaccgcctactatgcaaagcgtgaggctttcgggcaacctaagagatacacacatcgacttggtggaggtcaggttgattaccttaattggatagcagagcaaattggtgcaccgcccggtgaacaatggagatatcaggaaataaatggcggatactacagacttgctacacaatcagacactttccgtgataagtgggacgatgatcatctcatagttgaggcttatgaggatttcttgagacagaagctgattagtagtcttccttctcagttattggaatcttga

SEQ ID NO: 2 - protein, 459 aa, Arabidopsis thaliana
MAPARTRVNSLNVAVIGAGAAGLVAARELRRENHTVVVFERDSKVGGLWVYTPNSEPDPLSLDPNRTIVHSSVYDSLRTNLPRECMGYRDFPFVPRPEDDESRDSRRYPSHREVLAYLEDFAREFKLVEMVRFKTEVVLVEPEDKKWRVQSKNSDGISKDEIFDAVVVCNGHYTEPRVAHVPGIDSWPGKQIHSHNYRVPDQFKDQVVVVIGNFASGADISRDITGVAKEVHIASRSNPSKTYSKLPGSNNLWLHSMIESVHEDGTIVFQNGKVVQADTIVHCTGYKYHFPFLNTNGYITVEDNCVGPLYEHVFPPALAPGLSFIGLPWMTLQFFMFELQSKWVAAALSGRVTLPSEEKMMEDVTAYYAKREAFGQPKRYTHRLGGGQVDYLNWIAEQIGAPPGEQWRYQEINGGYYRLATQSDTFRDKWDDDHLIVEAYEDFLRQKLISSLPSQLLES

SEQ ID NO: 3 - DNA, 1552 bp, Brassica rapa
gcacgaggcaaaaaaacaaacataacattaacatttgaaaaatggcaccagctcaaaacctagtcagttcgaaacacgtagcggtgatcggagccggagcatccgggttaatagcggccagagagctccatcgtgaaggtcacaccgtcgtcgtttttgagcgggagaaacaagtgggaggtctctggatttactcacctaaatctgaatccgacccgcttggtctcgacccgacaagacctatagttcactcgagtgtctacgagtctctccgaaccaacctcccgagagagtgtatgggtttcagggattttccgttcgtgccatgtgttgatgacttttcaagagactcgagaaggtatccgagccacagggaagttcttgcgtaccttcaagactttgctagagagtttaaaatagaggagatggtccggttcgagaccgaggtggttcgggttgagccggttgatggaaaatggagggtccgatccaaaaactccgatgatctctccgaagatgagatctttgacgcagtcgttgtttgcagtgggcattataccgaaccttatgttgctcatattcctgggataaaatcatggccaggaaagcagatccatagccataactacagagttccgggtccattcaaaaatgaggtggtggtggtcatcggaaattttgcgagcggtgccgatattagtagagacgtagctaaggtcgccaaagaagtccacgttgcgtctagagggagtgaagctagtacgtatgagaagctttccgtgcccaccaacaatctatggattcattctgagatagagactgcatgtgatgatggttcaattgttttcaaaaatgggaaggcggttcatgcagatactgttgtgtattgtaccgggtacaagtataagtttccatttcttgaaaccaatggttatatgagcattgatgataaccgcgttgaacctttgtacaaacatgtctttccaccggcgcttgccccagggctttcttttgttggtttaccagggatgggcatacaattcgtcatgtttgaaatccaaagcaaatgggtagctgcagttttgtctggacgagttacacttcctgcaccagaaaaaatgatggaagatcttattgcatcgtatgccatgcttgaagcgttaggtattcccaagagatatacacataaattgggtaaaattcagtctaattatcttgactgggtcgcagaagaatgtggttgtcagcctgttgagccttggagaactcaacaagttgaccgtggttatgagagacttgtttctaaccctgaaaattaccgcgatgaatgggacgacgatgatctcataaaagaagcgtacgaggattttgctagtaagaagttgattagctttcttccttcttatttccccaaatcaggaagatgatatcccataatggtgcctacttgtttttaagggtctacttgtattatttttaaaaatgttggttttaataaagctgaatgtaagggttgcttgttatacaatggctactacttttccctcgtgcc

SEQ ID NO: 4 - protein, 461 aa, Brassica rapa
MAPAQNLVSSKHVAVIGAGASGLIAARELHREGHTVVVFEREKQVGGLWIYSPKSESDPLGLDPTRPIVHSSVYESLRTNLPRECMGFRDFPFVPCVDDFSRDSRRYPSHREVLAYLQDFAREFKIEEMVRFETEVVRVEPVDGKWRVRSKNSDDLSEDEIFDAVVVCSGHYTEPYVAHIPGIKSWPGKQIHSHNYRVPGPFKNEVVVVIGNFASGADISRDVAKVAKEVHVASRGSEASTYEKLSVPTNNLWIHSEIETACDDGSIVFKNGKAVHADTVVYCTGYKYKFPFLETNGYMSIDDNRVEPLYKHVFPPALAPGLSFVGLPGMGIQFVMFEIQSKWVAAVLSGRVTLPAPEKMMEDLIASYAMLEALGIPKRYTHKLGKIQSNYLDWVAEECGCQPVEPWRTQQVDRGYERLVSNPENYRDEWDDDDLIKEAYEDFASKKLISFLPSYFPKSGR

SEQ ID NO: 5 - DNA, 1526 bp, Cucumis sativus
gaaaacatgaataaacgaatcttatcataatttgcaaaaatcgaaaccaaattagttgacaaccacatcgaacaagaatcatcaataatccaattcccttttctaatcggaaaatcaaacggatgttatctcctctcaatttcctcccaacttcccgccgcgtggcagtaatcggcgccggtgccggtggcctcgtcactgcccgtgagctcggccgcgagggccaccatgtcgtcgttttcgaacgtaatactcgaatcggagggacctgggtatattcctcagagattgaatccgacccacttggactcgacccaaatcggacccgaattcacagcagtctctacaaatctctacgcaccaatctccccagagaactcatgggggtccgcgattacccttttgttcctcgagaaggggaggatcgagatcccaggcgatttccaagtcaccgggaggttctgaagtatttagaagatttcgctaatgaatttgggatttgtaaattggtgagatttggaactgaggtggtatttgctggtctggaggaggttgggaaatggaggattgaatttagatgtgaaaatggggatgttgaagaagacctttttgatgctctggttgtttgtgttggcaattattcacagcctcgagtggcagagattcctgggattgatggatggcctggggagcaagtgcatagtcacaattatcgtgatcccgaaccatttcggggtaaggttgttgtcttgataggttattcttcgagtggtacagacatttctcaggagctcattggggttgccaaagaaattcatattgcttggagatcaactaaaacagagcttttgaacacagaatcaattaacagtaatgtgtcatttcatccaatgattgaaagtgtccataaagatggggcagtggtttttcaagacgggtgcgttgttttggctgatattattctgcattgcactgggtacaaatatcatttcccttttcttgaaaccaatggcattgttacggtggacaacaaccgtgtaggacccctatacaagcatgtcttccccccagcattggccccagggctttcctttgttgggttaccatttaaggctgttcctttgcccatctttgagcttcaaagcaattggattgctggtgttttatcaaacaggattgcacttccatcaaaagaggaaatgttggcagatgttaaagctttctatgaaaatcttgaagcttttgggaagcccaagcatcggacccatgaattgggtgatgatatgcctgtgtattgtaactggcttgcaacaacttgtggttgtccagcctttgaagaatggaggaagaaaatgtacattgctattggtatttataaaaaggccaatctcgagacatatcgtgatgattggcaggacaatgagttgattcgtcaagcttacgaggaattcagcaagtataaatacaaatgaaaggacactcaaaaccacatagttttgaatgcttcataagattggttctatatg

SEQ ID NO: 6 - protein, 449 aa, Cucumis sativus
MLSPLNFLPTSRRVAVIGAGAGGLVTARELGREGHHVVVFERNTRIGGTWVYSSEIESDPLGLDPNRTRIHSSLYKSLRTNLPRELMGVRDYPFVPREGEDRDPRRFPSHREVLKYLEDFANEFGICKLVRFGTEVVFAGLEEVGKWRIEFRCENGDVEEDLFDALVVCVGNYSQPRVAEIPGIDGWPGEQVHSHNYRDPEPFRGKVVVLIGYSSSGTDISQELIGVAKEIHIAWRSTKTELLNTESINSNVSFHPMIESVHKDGAVVFQDGCVVLADIILHCTGYKYHFPFLETNGIVTVDNNRVGPLYKHVFPPALAPGLSFVGLPFKAVPLPIFELQSNWIAGVLSNRIALPSKEEMLADVKAFYENLEAFGKPKHRTHELGDDMPVYCNWLATTCGCPAFEEWRKKMYIAIGIYKKANLETYRDDWQDNELIRQAYEEFSKYKYK

SEQ ID NO: 7 - DNA, 1541 bp, Cucumis sativus
atggaattcatcgctacttgccaccctgactttcctccccctccggcctcacctcaacccacgacgatgcaacactcccgccgcgtggcagtgatcggcgccggtggcgcaggcctcatctccgcccgccaactttcccgggagggccaccaagtcgtggtcttcgaacggaataatcagatcggaggggtctgggtatattcgcccgaaattgaatccgacccacttggagttcaccctaagcggactcgaatacatagcagcctctacaaatctctacgaaccaatatccccagagaagtcatgggggtccgtgatttcccctttgttcctcgagaaggggaggatcgagatcccaggcgatttccaagtcaccgggaggttctgaagtatttagaagatttcgctaatgaatttgggatttgtaaattggtgagatttagaactgaggtggtgtttgctggtttggagaagcttggcaaatggagggttgaattcagatgtgagaatggggatgttcattatgacatttttgatgctgtagttgtttgtgttggcaatttttcgcagcctcgagtagcagagattccagggattgatggatggcctggggagcaagtgcatagtcacaattatcgtgatcccgaaccatttcgcggtaaggttgttgtgttgataggttattcttcgagtggtacggacatttctcaggagctcattggggttgccaaagaaattcatattgcttgcaggccagctaaaacagagtcttcggacgaaaaatcaattattagtaacgtctcatttcatccaatgatcgaaagtgtccataaagatggaacggtggtctttcaagacgggtccgtcgtttcggctgatgttattctgcattgtactgggtacaaatatcatttcccgtttcttgaaaccaatggcactgttacggtggacgacaaccgtgtaggacctcttttcaagcatgtcttccccccagcattggccccagggctttccttcgttgggttaccatttaaggttgttccttttgtcatatttgagcttcaaagcaattggattgctggtgttttatcaaacaggattgcacttccatcaaaagaggaaatgttggcagatgttaaagctttttatgaagaactcgaagctcgtggcaagcccaagcatcggacccataaattgggtggttatacgcctgcctactgtaactggcttgcagcaacttgtggttgtcctccctatgaagaatggagaaaggaaatgtttgttgctactgatattaataaagtggccaatcttgagtcataccgtgatgattggcatgacgatgagttgattcatcaagcttatgaagaatttggcaagtatactactacaaatgaaggaagtcaaaaccactcgaatttgaatgtttaataagtttggttctatatatttgtacattgcacaatcatgtgtcttgattataaatgttggatcttgatttataaataaaaatgaaaataatattagaccagattatgaca

SEQ ID NO: 8 - protein, 476 aa, Cucumis sativus
MEFIATCHPDFPPPPASPQPTTMQHSRRVAVIGAGGAGLISARQLSREGHQVVVFERNNQIGGVWVYSPEIESDPLGVHPKRTRIHSSLYKSLRTNIPREVMGVRDFPFVPREGEDRDPRRFPSHREVLKYLEDFANEFGICKLVRFRTEVVFAGLEKLGKWRVEFRCENGDVHYDIFDAVVVCVGNFSQPRVAEIPGIDGWPGEQVHSHNYRDPEPFRGKVVVLIGYSSSGTDISQELIGVAKEIHIACRPAKTESSDEKSIISNVSFHPMIESVHKDGTVVFQDGSVVSADVILHCTGYKYHFPFLETNGTVTVDDNRVGPLFKHVFPPALAPGLSFVGLPFKVVPFVIFELQSNWIAGVLSNRIALPSKEEMLADVKAFYEELEARGKPKHRTHKLGGYTPAYCNWLAATCGCPPYEEWRKEMFVATDINKVANLESYRDDWHDDELIHQAYEEFGKYTTTNEGSQNHSNLNV

SEQ ID NO: 9 - DNA, 1570 bp, Cucumis sativus
atgtggagtagagttggtagcacttcagctatcattcatacttttatcaaaaaagattcccatttcttacccataaatccatgttctactcaattagctacactcaatttcctcccctcccctcaaccatcaacgatgcctcactccagccgcgtggcagtgatcggcgccggcgccggaggcctcgtctcagcccgggaactttcccgggaggaccaccatgtggttgtattcgaacggaatactcaaattggaggggcctgggtatattcaccggaaattgaatccgacccacttggagtcgacccggatcggacccgaatccatagcagcctcttcaaatctcttcgaaccaatatacctagagaactcatgggggtccgggatttcccgtttgttcctcgagaaggggaggatcgagatccgaggcgatttccaagtcatcaggaggttcgcaagtatttggaagatttcgctaatgaatttggggtttacaaatttgtgagatttggaactgaggttgtgtttgctggtttggaggagcttgggaaatggaggattgaatttagatgtgaaaatggggacgttgattatgagatttttgatgctgtggttgtttgtgttgggaattattcgcagcctcgagtagcagagattcctgggattgatggatggcctggagagcaagtgcatagtcacaattatcgtgatcccgaaccatttcggggtaaggttgttgtgttgataggttattcttcgagtggaacagacatttctcaggagctcattggggttgccaaagaaattcatattgtttggagatcacctaaaacagagcttttggacagagaatcaattattagtaatgtttcatttcatccaatgattgaaagtgtgtgtaaagatgggacagtggtctttcaagacgggtgtgttgtttcggctgatgtaattttgcattgcactgggtacaactatcatttccctttccttgaaaccaatggcaatgttacagtggacgacaaccgtgtaggacctctatacaagcatgtcttccccccagcattggccccggggctttcctttgttggattaccattcaaggttattccttttcccttgtttgagcttcaaagcaattgggttgctggtgttttatcaaaaaggattgcacttccatcaaaagaggaaatgttggcagatgttaaagctttctatgaagatcttgaagctcttggcaagcccaagcatcggacccatttattgggtgattatatgatgcctgcctattgtaattgggttgcaacaacttgtggttgtcctccctatgaagaatggagaaaggaaatgaacatttctgttcatctttatagattgcccaatctcaagacgtaccgtgatgattggcacgatgatgagttgattcgtcaagcttacgaggagtttagcaagtataatacaaatgtaagaagtcaaaacaactcaaatttgaatgcttcataagatttgttgtatatgtgtacatttacatatttatgttgtcattgatccttcctcctcgttacaaata

SEQ ID NO: 10 - protein, 500 aa, Cucumis sativus
MWSRVGSTSAIIHTFIKKDSHFLPINPCSTQLATLNFLPSPQPSTMPHSSRVAVIGAGAGGLVSARELSREDHHVVVFERNTQIGGAWVYSPEIESDPLGVDPDRTRIHSSLFKSLRTNIPRELMGVRDFPFVPREGEDRDPRRFPSHQEVRKYLEDFANEFGVYKFVRFGTEVVFAGLEELGKWRIEFRCENGDVDYEIFDAVVVCVGNYSQPRVAEIPGIDGWPGEQVHSHNYRDPEPFRGKVVVLIGYSSSGTDISQELIGVAKEIHIVWRSPKTELLDRESIISNVSFHPMIESVCKDGTVVFQDGCVVSADVILHCTGYNYHFPFLETNGNVTVDDNRVGPLYKHVFPPALAPGLSFVGLPFKVIPFPLFELQSNWVAGVLSKRIALPSKEEMLADVKAFYEDLEALGKPKHRTHLLGDYMMPAYCNWVATTCGCPPYEEWRKEMNISVHLYRLPNLKTYRDDWHDDELIRQAYEEFSKYNTNVRSQNNSNLNAS

SEQ ID NO: 11 - DNA, 1590 bp, Cucumis sativus
aacatgaataaacgaatcttatcataatttgcaaaaatcgaaaccaaattagttgacaaccacatcgaacaagaatcatcaataatccaattcccttttctaatcggaaaatcaaacggatgttatctcctctcaatttcctcccaacttcccgccgcgtggcagtaatcggcgccggtgccggtggcctcgtcactgcccgtgagctcggccgcgagggccaccatgtcgtcgttttcgaacgtaatactcgaatcggagggacctgggtatattcctcagagattgaatccgacccacttggactcgacccaaatcggacccgaattcacagcagtctctacaaatctctacgcaccaatctccccagagaactcatgggggtccgcgattacccttttgttcctcgagaaggggaggatagagatccgaggcgatttccaagtcaccgggaggttctgaagtatttagaagatttcgctaatgaatttgggatttgtaaattggtgagatttggaactgaggtggtatttgctggtctggaggaggttgggaaatggaggattgaatttagatgtgaaaatggggatgttgaagaagacctttttgatgctctggttgtttgtgttggcaattattcacagcctcgagtggcagagattcctgggattgatggatggcctggggagcaattacatagtcacaattatcgtgatcccgaaccatttcggggtaaggttgttgtcttgataggttattcttcgagtggtacagacatttctcaggagctcattggggttgccaaagaaattcatattgcttggagatcaactaaaacagagcttttgaacacagaatcaattaacagtaatgtgtcatttcatccaatgattgaaagtgtccataaagatggggcagtggtttttcaagacgggtgcgttgttttggctgatattattctgcattgcactgggtacaaatatcatttcccttttcttgaaaccaatggcattgttacggtggacaacaaccgtgtagggcccctatacaagcatgtcttccccccagcattggccccagggctttcctttgttgggttaccatttaaggttgttccttttcccttgtttgagcttcaaagcaattggattgctggtgttttatcaaacaggattgcacttccatcaaaagaggaaatgttggcagatgttaaagctttctatgaaaatcttgaagcttttgggaagcccaagcatcggacccatgaattgggtgatgatatgcctgcctacttggactggcttgcagcagtatgtggttgtcctgcctatgaagaatggagaaaggaaatgtacattgctactcatatgaataaagtggccaatctcaggtcataccgtgacgattggcacgacaatgagttgattcgtcaagcttatgaagaatttagcaagtatgcaacaaatgaaggaagtgggaaccactcaaaattgagtgtttgataagattggttgtatacatgttacataatttatgtgttgttgattaatgaaaataatagtagtatgggatcgcccattttctttacaa

SEQ ID NO: 12 - protein, 460 aa, Cucumis sativus
MLSPLNFLPTSRRVAVIGAGAGGLVTARELGREGHHVVVFERNTRIGGTWVYSSEIESDPLGLDPNRTRIHSSLYKSLRTNLPRELMGVRDYPFVPREGEDRDPRRFPSHREVLKYLEDFANEFGICKLVRFGTEVVFAGLEEVGKWRIEFRCENGDVEEDLFDALVVCVGNYSQPRVAEIPGIDGWPGEQLHSHNYRDPEPFRGKVVVLIGYSSSGTDISQELIGVAKEIHIAWRSTKTELLNTESINSNVSFHPMIESVHKDGAVVFQDGCVVLADIILHCTGYKYHFPFLETNGIVTVDNNRVGPLYKHVFPPALAPGLSFVGLPFKVVPFPLFELQSNWIAGVLSNRIALPSKEEMLADVKAFYENLEAFGKPKHRTHELGDDMPAYLDWLAAVCGCPAYEEWRKEMYIATHMNKVANLRSYRDDWHDNELIRQAYEEFSKYATNEGSGNHSKLSV

SEQ ID NO: 13 - DNA, 1600 bp, Medicago truncatula
atgaatgaacatgttcatactgtaagcattcaattcgattccaagccaatgaaattcatcatgtccaccgcaacaccacttctcacaccccgccacgtggcagtcatcggagccggcgccggaggcttagtagcagcacgcgagctccgacgagaaggacatcaagtagtagtcttcgagcgaggagaagaattgggcggttcatgggtctacacttcagaggtagaatccgacccactcggtttggacccgaaccggaagcttatccactcgagcctatacaattcactccgaaccaatttgcctcgggagagtatgggtttccgagattacccttttaggaggaaagaagagaaggggagagattctagaaggttcccgagtcatggagaggtattgatgtatttgaaggattttgctgcggattttgagattagtgatttggtgaggttgaagacagaggtggtgtttgctggggtgggtgaaggtggaaaatggacggtgagatctagatcagtggagagagaatgtgtggatgagatttatgatgctgttgttgtttgcaatggacattattttcaaccaagacttcccaatattcctggcattaatgcatggccagggaagcaaatgcatagccataattacagaacacccgagccctttcaagatcaagttgtagttctaattggtggtgctgccagtgcggttgatatttctcgagacgtggcaaccgttgctaaagaagttcatattgcagctaggtctgttgaagaagataagcttggaaagttacctggccatgataacatgtggcttcattctatgattgacagtgttcatgaagatggtgcagtggtttttaaagatggaaatgcagttatcgctgacttcattgtacattgcacagggtacaagtatgattttcctttccttgaaaccaacagcgtggtgactgtagatgacaatcgtgttggaccactctacaagcatgtttttccaccggcgttagctccatggctttcctttgttgggttaccttggaaggttgctcccttccctttgtttgaattgcagagtaagtggatagctggagttttgtctaatcgcattgcccttccttcagaagaggagatgactaaagatattgaagctttttacttgtcacttgaagaatctggcattcctaagaggcacactcataatatgggcacgggcacggccgatgttcagtgggactacaataactggcttgcagatcagtgtggtgttcctgctatggaagaatggagaaggcaaatgtatatggctacatcgaagaacaggctcttgcgacctgagacttatcgtgatgagtgggacgatgatgacattgttcaactagctgagcatgaatttgctaagtatcagatataatgttgtattgttttgagatttaccaagtacaagtcattcatgcgttatacgcctagttcagtgttattcttaacgatcaaaaatcagctttaaagtgcaaataagaatgtaaattatatatgtttggaatactttcaataattcattaatgaacatgtgataatgatgtgatctctttttattt

SEQ ID NO: 14 - protein, 471 aa, Medicago truncatula
MNEHVHTVSIQFDSKPMKFIMSTATPLLTPRHVAVIGAGAGGLVAARELRREGHQVVVFERGEELGGSWVYTSEVESDPLGLDPNRKLIHSSLYNSLRTNLPRESMGFRDYPFRRKEEKGRDSRRFPSHGEVLMYLKDFAADFEISDLVRLKTEVVFAGVGEGGKWTVRSRSVERECVDEIYDAVVVCNGHYFQPRLPNIPGINAWPGKQMHSHNYRTPEPFQDQVVVLIGGAASAVDISRDVATVAKEVHIAARSVEEDKLGKLPGHDNMWLHSMIDSVHEDGAVVFKDGNAVIADFIVHCTGYKYDFPFLETNSVVTVDDNRVGPLYKHVFPPALAPWLSFVGLPWKVAPFPLFELQSKWIAGVLSNRIALPSEEEMTKDIEAFYLSLEESGIPKRHTHNMGTGTADVQWDYNNWLADQCGVPAMEEWRRQMYMATSKNRLLRPETYRDEWDDDDIVQLAEHEFAKYQI

SEQ ID NO: 15 - DNA, 2747 bp, Oryza sativa
catgcctacgcagcctcatgtccagtcgagtgtaaccacaagcccacgggaatttgctgtccaatgaagaccccacaaaacgacaaactccaataccacacacctccgcttccctccaaatcgcaacagaaatccgaagcgaaatcgcgcacgcaccgtctcgcgatgccgtccccgtcgctccgcctcgccgtcgtcggcgcgggcgccgccggcctggtggcggcgcgggagctccgccgggagggccactcccccgtggtgttcgagcgcgccgcctccgtgggcggcacgtggctctacgacgccgcccccgccacctccgacccgctcgccgccggcgccgcccactccagcctctacgcctcgctccgcaccaacctgccgcgggaggtgatgggcttcctcgactttcccttcgcctcctccgccgcggaggccggcggcggcggcgacacgcgcaggttccccggccacgacgaggtgctccggtatctggaggagttcgcgcggcggttcgacctgtacggcctcgtccgcttcgggacggaggtggttagggttcggagggatggcggcggcggcggcgggaggtgggcggtgacgtcgaggaagatcggggagaaggggaggcgtgaggaggaggaggaggtgtatgatgccatcgtggtttgcaatggccattacacggagcctcgcgtcgcccacatacctggtaatctcctcgtcactagcaaagttgcaatctaatcaattttgcttgaactactcctaatcatggacagattcaaggaattaaataatttggtacttcgtactgttgccaatgtatggttgcaatgttgtgatcgtcgaatccaggaaggttaatttaactccaaattgaactcaaccgtttgaagttttgaaaatagattgggattgatttcaaatctgtatttttttaacactgttatgatgatcatcaaatccaggaaggttaagtaaactcaaaattaaactcaactgttaggttgggattgacttcaaatatgtattctttaaacactgacaggggtggaggcttggcctggaaagcagatgcatagccacaattaccgcgttccagagcctttccacgatcaagtaactgtctttctttacctgtgcaatctttcctatcatgcatttgtgcttaaatgttatcttggttgatgcgttgcacttgtaggtagtgatcataatcggggcatcagcaagtgcagtagacatctcaagggaccttgcaggtgttgcagaagaggttcatgttgctgatagatcagcacctgcctgcacttgcaaaaggcagcctggatatgataatatgtggctccattccatggtaaacgcccttttctcgtggtgagtgatagcatatggtagctttatccgctgaaagggctgccacatttagcacaactagaaaactaattttcaagctgcagattgatcatgcacaagaagatggctgcgtggtgtttcaggatggcagctcaatcaaagccgatgtcatcatgcactgtactgggtatgtaaacctgcactaccctgcaacccatttctcggcttcttgtgcgaaattgcatttttgttactacctccgtttcaggttatgcctagattcattaatatcaatatgaatatgagcaatgctagaaagtcttataacctaaaacggaggaagtacttcagttgaaactaacaatgtgttcctttcatctgcctgtcgactagctacttgtatgattttccattccttgaggatgatagcgccatcaccgttgatgacaactgtgtcgatccactatacaagcacgttttcccaccagaagtagcacctcacctgtccttcatcggattgccatggaaggtcattatgtgtgcaaaaagtgatgccattcactttagatgcacttgtaattaagttgtcttgatcttgtgatgatcataggatgtaaagcattcccctgccttgcttcttgcaggtcattccttttccattgtttgaactccaaagcaaatgggttgccggcgtgctatcaggacgagtcaagcttccttcgagcgaagaaatgatggaagatgtgaaagccttccactcgaaaatggaagcgcgtggatggcctaagagatacgcccacaacttttcagactgtcaggtagcctggagatgctttgagtgtcagttaccaaagttctaatgttttgaaacgaaattaataaatatgattgtcatctacctgcaatttttcagtagtttcgtttatgctcccttgataagcttgttttcatttccagtttgaatatgatgattggcttgcggagcaatgtggccatccaccaattgaacaatggaggaagctgatgtatgctgctaattcagagaacaaggctgctcgtccggagagttaccgcgatgagtgggacgatgatcatcttgtggcagaagcagcagaagatttcaagaaatacttgtaaaatctcaagaagatttcattcaatgtacatgattgcaaatttgcaatgcagaaaacatcagagaataattctgtacacccaaaatctcaattcatgtctggaatgggcacaaatgctcgtcatcagatagttggtttacttgtgtattatttgatcatttgatgcctgtagattgtaataataacctgaagcaaaaacaagagaataattctgtgcatgagaaaggagaaaccttgagtctggaacgg

SEQ ID NO: 16 - protein, 482 aa, Oryza sativa
MKTPQNDKLQYHTPPLPSKSQQKSEAKSRTHRLAMPSPSLRLAVVGAGAAGLVAARELRREGHSPVVFERAASVGGTWLYDAAPATSDPLAAGAAHSSLYASLRTNLPREVMGFLDFPFASSAAEAGGGGDTRRFPGHDEVLRYLEEFARRFDLYGLVRFGTEVVRVRRDGGGGGGRWAVTSRKIGEKGRREEEEEVYDAIVVCNGHYTEPRVAHIPGVEAWPGKQMHSHNYRVPEPFHDQVVIIIGASASAVDISRDLAGVAEEVHVADRSAPACTCKRQPGYDNMWLHSMIDHAQEDGCVVFQDGSSIKADVIMHCTGYLYDFPFLEDDSAITVDDNCVDPLYKHVFPPEVAPHLSFIGLPWKVIPFPLFELQSKWVAGVLSGRVKLPSSEEMMEDVKAFHSKMEARGWPKRYAHNFSDCQFEYDDWLAEQCGHPPIEQWRKLMYAANSENKAARPESYRDEWDDDHLVAEAAEDFKKYL

SEQ ID NO: 17 - DNA, 1552 bp, Vitis vinifera
ctctactatcttcccatggcgccctccatctccctgttcaaatcacgtgacgttgccgtcatcggggctggcgctgccggtttagtcgccgcccgtgagctccgccgtgaaggccacaaggtcgttgtcttcgagcgggaacgccaagtgggtgggacctgggtctacacgcccacagtggagacggatccacttggctccgacccgtctcgacacatagtccactccagcctctacgcctccctccgcaccaacctccctagagaggtcatgggttttctggactaccccttcgtatccactggtgaaccacatagggaccccagaaggtttccgggtcaccgagaggtctcgctttatctcaaggattttgcggttgggtttggactcaatgaattaatccgcttcgagacggaggtagtttatgctggtttggtcgaggatgagaagtggagggtgaagtctagaagcggaaacgatgcggcaattgatgtggaggagatttttgatgctgtggttgtttgcaatggccattacacagagccccgtcttgcagaaattcctggcattgatgcatggccaggaaagcatatgcatagtcacaattatcgtattcctgagccctttcgagatcaggttgtagttttgatagggggtgctgcaagtgctgtcgacatctctatggacattgctcaagttgctaaagcagttcatattgcatctagatcagttgaggctggaatcttgaaaaagttatctggcaatgccattgataacatgtggcttcatcctatgatagaaagtgtccagaaagatggtactgtgatattttatgatgggagtgtggttcttgctgatgtaattctgcactgcacgggatacaagtatcatttcccttttcttgacaccagtggaattgtgactgtggatgacaatcgtgtgggacctctatacaagcatatttttccaccacatttggctccagggctttcctttgttggtttgccatggaaggtcctccctttccccatgtttgaattccaaagcaaatggatagcaggtgctctctcaggtcggattggactcccatcgcaggaggagatgatggcagatgtttcagccttttatttgtcactagaagcttctgacacaccaaagcactacactcacaacttggctgattctcaggtaaatttgaactcttatataagtgggttaggatactgtcatgttcatttttcttactggttatctctcaaagtaatgttgaaactgttcttggatggcattttgcagtttgagtatgatgattggcttgccttggaatgcgggattccaggcgttgaagaatggagaaagaaaatgtatgaagcaactgccaagaacaagaaggtccgaccagacaaataccgcgacaaatgggaagatgaagacttaatgttggaagctcagaaggacttcgctggatgccgcctgaatggggctggtgacaattgaaaccaacctcccctacaaaataagcaatgaaaaaaaaaaaaaagcccgataaaga

SEQ ID NO: 18 - protein, 493 aa, Vitis vinifera
MAPSISLFKSRDVAVIGAGAAGLVAARELRREGHKVVVFERERQVGGTWVYTPTVETDPLGSDPSRHIVHSSLYASLRTNLPREVMGFLDYPFVSTGEPHRDPRRFPGHREVSLYLKDFAVGFGLNELIRFETEVVYAGLVEDEKWRVKSRSGNDAAIDVEEIFDAVVVCNGHYTEPRLAEIPGIDAWPGKHMHSHNYRIPEPFRDQVVVLIGGAASAVDISMDIAQVAKAVHIASRSVEAGILKKLSGNAIDNMWLHPMIESVQKDGTVIFYDGSVVLADVILHCTGYKYHFPFLDTSGIVTVDDNRVGPLYKHIFPPHLAPGLSFVGLPWKVLPFPMFEFQSKWIAGALSGRIGLPSQEEMMADVSAFYLSLEASDTPKHYTHNLADSQVNLNSYISGLGYCHVHFSYWLSLKVMLKLFLDGILQFEYDDWLALECGIPGVEEWRKKMYEATAKNKKVRPDKYRDKWEDEDLMLEAQKDFAGCRLNGAGDN

SEQ ID NO: 19 - DNA, 1444 bp, Vitis vinifera
ctctactatcttcccatggcgccctccatctccctgttcaaatcacgtgacgttgccgtcatcggggctggcgctgccggtttagtcgccgcccgtgagctccgccgtgaaggccacaaggtcgttgtcttcgagcgggaacgccaagtgggtgggacctgggtctacacgcccacagtggagacggatccacttggctccgacccgtctcgacacatagtccactccagcctctacgcctccctccgcaccaacctccctagagaggtcatgggttttctggactaccccttcgtatccactggtgaaccacatagggaccccagaaggtttccgggtcaccgagaggtctcgctttatctcaaggattttgcggttgggtttggactcaatgaattaatccgcttcgagacggaggtagtttatgctggtttggtcgaggatgagaagtggagggtgaagtctagaagcggaaacgatgcggcaattgatgtggaggagatttttgatgctgtggttgtttgcaatggccattacacagagccccgtcttgcagaaattcctggcattgatgcatggccaggaaagcatatgcatagtcacaattatcgtattcctgagccctttcgagatcaggttgtagttttgatagggggtgctgcaagtgctgtcgacatctctatggacattgctcaagttgctaaagcagttcatattgcatctagatcagttgaggctggaatcttgaaaaagttatctggcaatgccattgataacatgtggcttcatcctatgatagaaagtgtccagaaagatggtactgtgatattttatgatgggagtgtggttcttgctgatgtaattctgcactgcacgggatacaagtatcatttcccttttcttgacaccagtggaattgtgactgtggatgacaatcgtgtgggacctctatacaagcatatttttccaccacatttggctccagggctttcctttgttggtttgccatggaaggtcctccctttccccatgtttgaattccaaagcaaatggatagcaggtgctctctcaggtcggattggactcccatcgcaggaggagatgatggcagatgtttcagccttttatttgtcactagaagcttctgacacaccaaagcactacactcacaacttggctgattctcagtttgagtatgatgattggcttgccttggaatgcgggattccaggcgttgaagaatggagaaagaaaatgtatgaagcaactgccaagaacaagaaggtccgaccagacaaataccgcgacaaatgggaagatgaagacttaatgttggaagctcagaaggacttcgctggatgccgcctgaatggggctggtgacaattgaaaccaacctcccctacaaaataagcaatgaaaaaaaaaaaaaagcccgataaaga

SEQ ID NO: 20 - protein, 457 aa, Vitis vinifera
MAPSISLFKSRDVAVIGAGAAGLVAARELRREGHKVVVFERERQVGGTWVYTPTVETDPLGSDPSRHIVHSSLYASLRTNLPREVMGFLDYPFVSTGEPHRDPRRFPGHREVSLYLKDFAVGFGLNELIRFETEVVYAGLVEDEKWRVKSRSGNDAAIDVEEIFDAVVVCNGHYTEPRLAEIPGIDAWPGKHMHSHNYRIPEPFRDQVVVLIGGAASAVDISMDIAQVAKAVHIASRSVEAGILKKLSGNAIDNMWLHPMIESVQKDGTVIFYDGSVVLADVILHCTGYKYHFPFLDTSGIVTVDDNRVGPLYKHIFPPHLAPGLSFVGLPWKVLPFPMFEFQSKWIAGALSGRIGLPSQEEMMADVSAFYLSLEASDTPKHYTHNLADSQFEYDDWLALECGIPGVEEWRKKMYEATAKNKKVRPDKYRDKWEDEDLMLEAQKDFAGCRLNGAGDN

SEQ ID NO: 21 - DNA, 1674 bp, Vitis vinifera
ctccactttcttcccatggcgccctccatctccctgttcaaatcacgtgacgttgccgtcatcggggctggcgctgccggtttagttgccgcccgtgagctccgccgtgaaggccacaaggtcgttgtcttcgagcgggaacgccaagtgggtgggacctgggtctacacgcccacagtggagacggatccacttggcgccgacccgtctcgacacatagtccactccagcctctacgcctccctccgcaccaacctccccagagaggtcatgggttttctggactaccccttcgtatccactggtgaacctcatagggaccccagaaggtttccgggtcaccgagaggtctcgctttatctcaaggattttgtggttgggtttggactcaatgaattaatccgcttcgagacggaggtggtttatgctggtttggttgaggatgagaagtggggagtgaagtctagaagcggaaacgatgcggcaattgatgtggaggagatttttgatgctgtggttgtttgcaatggccattacacagagccccgtcttgcagaaattcctggcattgatgcatggccaggaaagcatatgcatagtcacaattatcgtactcctgagccctttcgagatcaggttgtagttttgatagggagtgctgcaagtgctgttgacatctctatggacattgctcaagttgctaaagcagttcatattgcatctagatcagttgaggctggaatcttggaaaagttatctggcaatgctgttgataacatgtggcttcatcctatgatagaaagtgtccagaaagatggtactgtgatattttatgatgggagtgtggttcttgctgatgtaattctgcactgcacgggatacaagtatcatttcccttttcttgacaccagtggaattgtgactgtggatgacaatcgtgtgggacctctatacaagcatatttttccaccacatttggctccagggctttcctttgttggtctgctatggaaggtcctccctttccccatgtttgaattccaaagcaaatggatagcaggtgctctctcaggtcggattggactcccatcgcaggaggagatgatggcagatgtttcagccttttatttgtcacgagaagcttctgacacaccaaagcactacactcacaacttggctgattctcaggtaaatttgagctcttatataagtgggttaggatactgtcattttcatttttcttactggttatctctcaaagtaatgttgaaactgttcttggatgctattttgcagtttgagtatgatgattggcttgccttggaatgcgggattccaggcgttgaagaatggagaaagaaaatgtatcaagcaactgctaagaataagaaggtccgaccagacaaataccgcgacgaatgggaagatgaagacttaacgttggaagctcagaaggacttcgccagatgccgcccgaatgggggtggcgacaattgaaaccaacctcccctacaaaataagcaatgaaaaaaaaaaaaaagcccgataaagatggatctggatattgtcttggttgagtcattcattgttcttctcttgagacttgaggtatattaattgaataatcagccatagcgtagataatttttttttttatagcgattgttttgatcg

SEQ ID NO: 22 - protein, 457 aa, Vitis vinifera
MAPSISLFKSRDVAVIGAGAAGLVAARELRREGHKVVVFERERQVGGTWVYTPTVETDPLGADPSRHIVHSSLYASLRTNLPREVMGFLDYPFVSTGEPHRDPRRFPGHREVSLYLKDFVVGFGLNELIRFETEVVYAGLVEDEKWGVKSRSGNDAAIDVEEIFDAVVVCNGHYTEPRLAEIPGIDAWPGKHMHSHNYRTPEPFRDQVVVLIGSAASAVDISMDIAQVAKAVHIASRSVEAGILEKLSGNAVDNMWLHPMIESVQKDGTVIFYDGSVVLADVILHCTGYKYHFPFLDTSGIVTVDDNRVGPLYKHIFPPHLAPGLSFVGLLWKVLPFPMFEFQSKWIAGALSGRIGLPSQEEMMADVSAFYLSREASDTPKHYTHNLADSQFEYDDWLALECGIPGVEEWRKKMYQATAKNKKVRPDKYRDEWEDEDLTLEAQKDFARCRPNGGGDN

SEQ ID NO: 23 - DNA, 887 bp, Gossypium hirsutum
ctagatcagtggcggatgaaacgtatatgaaacagcctggttacgataatttgtggttccattccatgatagatcatgcacatgaggatggcatggtggttttccgaaatgggaaaacagtgcttgctgatctcattatgcactgcactgggtacaagtatcacttccctttccttgacacaaaaggcattgtgactgtggacgataatcgtcttggaccactatacaagcacgtctttcccccagccttagccccatacctttcatttattgggataccatggaagattgttcctttccccttatttgagtttcaaagcaaatggatagccggtattttgtccggtcgtattacacttccatcacaaaaggaaatgatggaagatattcaagcattttactcggcacttgaagattctagtataccaaaacggtatactcattgcattggtcaatctcaggttgaatacaataattggcttgctacacaatgtggttgccaaggtgttgaaaaatggagagaagcaatgtattctatggcttcggagaatcggcgtcttctaccagagatgtaccgtgatgaatgggatgatcaccacctggtttcagaagcttatgaggatttcattaagtacccttcagcatcaaacctttagagacaaaaaataaaaataaaaatcaatttacaatggtgaaggatgtcattcaccctgttgtatacaacccgggtctgtaccgtggcagggtactattctaccactagaccactggtgcttgtgcgatgaaagtctcgatatacataaatctagcacaagagttactaaacgaagagaattgaaggagaatcaactatatgaattttattgaataaaaaaaaaaaaaaaaaa

SEQ ID NO: 24 - protein, 217 aa, Gossypium hirsutum
RSVADETYMKQPGYDNLWFHSMIDHAHEDGMVVFRNGKTVLADLIMHCTGYKYHFPFLDTKGIVTVDDNRLGPLYKHVFPPALAPYLSFIGIPWKIVPFPLFEFQSKWIAGILSGRITLPSQKEMMEDIQAFYSALEDSSIPKRYTHCIGQSQVEYNNWLATQCGCQGVEKWREAMYSMASENRRLLPEMYRDEWDDHHLVSEAYEDFIKYPSASNL

SEQ ID NO: 25 - DNA, 1262 bp, Zea mays
atggaatgttgttgctgggctggctacagtacagtacaggtgcaggcttcattcctccagtggcggcggacaacgccgaacagacagaagaatactgtagagaagatcgtcgcacccaccggaggacgagacgggccctaggcacaccacgcaatcagccgccccgcgcccccgcccgcggttggcgatgttgccgtgacgtcttccagggaggagaagccaacatcacaaactccacgacgaataactggcagtagaagccgagaaggtaggcccgctcgttccaacatcacaaactccacacggctctcctgtctccgctgcccgctccacctccctccatgccgtcggcttccctccgcctcgccgtcgtcggcgcgggcgcggcgggcctggttgccgcccgcgagctacgccgcgagggccatgcgcccgtcgtcttcgagcgcgccgccgccgttgggggcacttggctctacacgcctcccgccacgtcctccgacccgctcggcgccgcggcgacgcattccagcctctacgcatcgctccgcaccaacctgccacgcgagaccatgggcttcctcgacttccccttcgccgctggcgccgcgggctcccgagacccccgccggtttcccgggcacgaggaggtgctccgctacctggaggcgttcgcgcgccggttcgacctgctccggctcgtccgcttcgagacggaggtgctcagtgtgaggagggaagacggagggaggtgggctgtgacgtcgaggaagctcggggataaggggagcggcgaggaggagttctatgatgccgtcgtggtctgcaatggtcactacacggagccacgcctcgccgtcattcccgtttgagtatgatgattggctcgctgagcaatgtggccatccaccagtcgaagaatggaggaagcagatgtatgctgtaacttcaatgaacaaggcagctcgtcctgagagttaccgtgatgaatgggatgacgagcatctggtggccgaagcaaatgaatacttcaagaaattcttgtaaattctttcttactattctcatcccatattctttcggcatacccgaggctgatctcaactgcaatatgcaaatatgaataaccatttaagtgatgtggattggatacacttctgggttagcatttcatcgatcattcgatcgatgtatatatatgagactgttctggtagtaaacaatcttgtagtaaactgtggattggtcatcaattaacaaca

SEQ ID NO: 26 - protein, 176 aa, Zea mays
MPSASLRLAVVGAGAAGLVAARELRREGHAPVVFERAAAVGGTWLYTPPATSSDPLGAAATHSSLYASLRTNLPRETMGFLDFPFAAGAAGSRDPRRFPGHEEVLRYLEAFARRFDLLRLVRFETEVLSVRREDGGRWAVTSRKLGDKGSGEEEFYDAVVVCNGHYTEPRLAVIPV

SEQ ID NO: 27 - DNA, 1359 bp, Populus trichocarpa
atgcaaacatcaaatgctacttcgctcacctctcgccacgtagctgtcatcggtgccggcgccgccggtttagtggcggcacgtgagctccggcgtgaaggtcaccaagtggttgtctttgagaaagatagccaaattggtgggacatgggtgtacactccacaggtcgaaaccgaccctcttgggctagacccgacccgacacatcgtccacaccagtttatacaagtccctccggaccaacttgccgagagagtcgatgggctttatggattatccattcgtgacccgagcgggtgaagggagcgaccctagaaggttcccgggtcatgcagaagtgttgaagtatctgcaagattttgcaagggagtttgggattgaagaaatggtgaggtttgagtgtgaagtggttagtgtggagatggttgataatgagaaattgaaagtgaagtgtaaaaggatgagacctgatggtggtgatgatgatctgctagatgaggtttttgatgctgttgttgtttgtaatggacatttcacataccctcgtattgctgaaatccctggcatcaacttgtggcccggaatgcaaatacatagccataactatcgtactcctgaactcttcaaggataaagttgtaattttaattggcagttctgcaagtgctattgatttatcccttgagattggtggaattgccaaagaggtgcacattgcatctagatcagttgccaatgatacatatgaaaagcgggctgaatgtgataatatatggctacattctatgataaaaagcgcacataaagatggttctgtggctttccgagatggtaacactatcgtcgctgatattattctgcattgcacagggtacaagtattacttcccattcctcaaaaccaatggcattgtgactgtggatgacaatcgtgttggaccactctacaagcatgttttcccacccatttttgccccgcagctttcctttgtcggactaccctacaggagtttacctttcccaatctttgaaattcaaagcaagtggatttctggtgttctatctgatcgaattgtgctcccttcacaagaggacatgatggaagatgttaacaccttctactcgacacttgaagattctggtgtgcctaagcatcacactcatagcatgggggacacaatgattgactacaatgcttgggttgcttctctgtgtcaatgtccttgctttgaagaatggagagtacaaatgttctatgaaacggccaagagattgaacgccaacccaaagacatttcgcgatgaatgggaagatgacaacctggtcttgcaagcctgtgaagatttcagcaaatacatctga

SEQ ID NO: 28 - protein, 452 aa, Populus trichocarpa
MQTSNATSLTSRHVAVIGAGAAGLVAARELRREGHQVVVFEKDSQIGGTWVYTPQVETDPLGLDPTRHIVHTSLYKSLRTNLPRESMGFMDYPFVTRAGEGSDPRRFPGHAEVLKYLQDFAREFGIEEMVRFECEVVSVEMVDNEKLKVKCKRMRPDGGDDDLLDEVFDAVVVCNGHFTYPRIAEIPGINLWPGMQIHSHNYRTPELFKDKVVILIGSSASAIDLSLEIGGIAKEVHIASRSVANDTYEKRAECDNIWLHSMIKSAHKDGSVAFRDGNTIVADIILHCTGYKYYFPFLKTNGIVTVDDNRVGPLYKHVFPPIFAPQLSFVGLPYRSLPFPIFEIQSKWISGVLSDRIVLPSQEDMMEDVNTFYSTLEDSGVPKHHTHSMGDTMIDYNAWVASLCQCPCFEEWRVQMFYETAKRLNANPKTFRDEWEDDNLVLQACEDFSKYI

SEQ ID NO: 29 - DNA, 1395 bp, Populus trichocarpa
atgcaagcatcaccgaatttgctctcctcccaccacgtcgccgtgatcggggcaggagccgccggtctggtggctgcacgtgagctccaccgagagggtcacaaagtggtggtctttgagaaagatgaccaagttggtggtctctggatgtacgatccccgtgtagaacccgaccctctcgggcttgacctaacccgacctgttgttcactcaagtctctacgagtctctcaggaccaacttgccaagggagacgatgggttttatggactacccgtttgtgacccgagagggtgagggaagagacccgagaaggtttccgggtcatagagaagtgttgatgtatttgcaggattatgccagggaatttgggattgaagagatggtaaggtttgggtgtgaggtggtgaatgtagagatgattgatagtgggaaatggaaagtgaagtcaaaaaggaagagacttgatgataatgatagaggtgatgattttgctgatcatgaggattttgatgctgttgttgtttgcgttggacattacacccaacctcgtatcgctgaaattcctggcatcaatttgtggccggggaagcagatacacagccacaactatcgtattcctgagcctttcagggatcaaatcataattttgataggagcttctgcgagcgctgctgatatatccgtggaaattgctggacttgccaaagaggttcacattgctcgtagatcggctgtagatgatgatacatacgaaaaaaagcctggatatgataacatatggcttcattccacgatagaaagagcatgtgaagatggtactgtcattttccgagatggcagtgttatcctagctgacgttattctgcactgcaccgggtacaaatatggcttcccttttctgaaaactgatggcatcgtgactgtggatgacaatcgtgtggggccattgtacaagcatgttttccctccaatcttggccccgtggctttcctttgtcgggataccctattggactttccctttcccaacgttcgaagttcaaagcaagtggattgctggtgttttatcaggtcgaattgctcttccttcacaagaggacatggtggaagatgttaagatctactactctgaacttgaagcttctggtgtacctaagcatcacactcataacttagctcattctacaaatgactacaacatgtggcttgcctcccagtgtcagtgttcatgctttgaagaatggagaattgaaatgtcccatgaaattcttaagaactggcgtgccaggccaaatatgtatcgtgacgaatgggacgacgaccacctgatcttgcaagcccatgaagacttcaacagacgcatctcaaacaaagccagtaatggtcatatctga

SEQ ID NO: 30 - protein, 464 aa, Populus trichocarpa
MQASPNLLSSHHVAVIGAGAAGLVAARELHREGHKVVVFEKDDQVGGLWMYDPRVEPDPLGLDLTRPVVHSSLYESLRTNLPRETMGFMDYPFVTREGEGRDPRRFPGHREVLMYLQDYAREFGIEEMVRFGCEVVNVEMIDSGKWKVKSKRKRLDDNDRGDDFADHEDFDAVVVCVGHYTQPRIAEIPGINLWPGKQIHSHNYRIPEPFRDQIIILIGASASAADISVEIAGLAKEVHIARRSAVDDDTYEKKPGYDNIWLHSTIERACEDGTVIFRDGSVILADVILHCTGYKYGFPFLKTDGIVTVDDNRVGPLYKHVFPPILAPWLSFVGIPYWTFPFPTFEVQSKWIAGVLSGRIALPSQEDMVEDVKIYYSELEASGVPKHHTHNLAHSTNDYNMWLASQCQCSCFEEWRIEMSHEILKNWRARPNMYRDEWDDDHLILQAHEDFNRRISNKASNGHI

SEQ ID NO: 31 - DNA, 1680 bp, Populus trichocarpa
tggggaccctgctgacgctagcatttctcaagtgtccaaaaaaccgaactctcttcgacaggtcaccacaacaacaccaatcaaacatttatcaatgccaccacctcaacttcctccaccaatctcccgccacgtggcggtgatcggtgccggagccgccggcctcgttagtgcccgtgagctccggagagagggtcatgatgttgtagtctttgaaagagacaaccaagtaggtggcacatgggtgtacaatccccgagtcgagcccgacccgttaagcctcgacccgaatcgacgcataattcactcgagcctctatagctccctccggaccaacctcccaagagaagtaatgggtttcaaagattatccctttatagcaaaaaatgataaaaagagagaccagagaaggtttccgggccatcgagaggtgttgttgtatttgcaggattttgcaagtgagtttgggattgaagaaatggtgaggtttgatactgaagtggttcatgtggggcctgttgaggataatattggaaagtggattgtgaggtctaaaaggaaaataagtgatgatgatagggaggttagttttggatttgatgttgacgaggagatttatgatgctgttgttatctgtaatggacattacactgaacctcgtattgctcaaataccagggatcagttcatggccaggaaaacagatgcatagccacaattatcgtactcctgagggctttcaagatcaagtggcaattttgattggaagttcagctagttctgatgatatatccagagaaattgctggagttgctaaagaggtccatgttgcctcaagatcagttgcggacgaaacatatcaagagcagcctggatatgataatatgtggcttcattctatgatagaaagtgtgcatgatgatggttctgtgatcttcagaaatgggagagttgtcgttgctgacattattctacattgcactgggtacaagtatcacttcccttttctagacaccaatggcattgtgaccatggatgaaaatcgtgtggcccccctgtacaagcaagtttttccaccagttctggccccatggctttcatttgttgggttaccgtggaaggttgtcccttttcccttggttgaacttcaaaccaagtggattgctggtgttttatcaggtcatattgcacttccgtcacctgaggagatgatggaagatgttaaagccttctatgagacactagaatcttccaacaaacccaaacactacactcataatttgggtggttgtcagttcgagtacgacaactggcttgcttctcagtgcggttgcccagggatcgaagaatggagaaggcaaatgtatgatgcagctagcaagagtaagcggctccggccagagatataccgtgatgaatgggatgatgatgacctggtcttggaagcctacggggacttcacaaagtacacttgaaaaagttgaagcaacagctgcatctctcaatggaggtgttcgaaggataggaaaggaagacgataaattattaggctggcctaattgtcaacatctgaatttgtgggatcaatcatcatcgttgttaatttggacttgtatttccatgattcgcagttctttacgtgaataaagaatcaatgaagatatccatatccatatatgaa

SEQ ID NO: 32 - protein, 459 aa, Populus trichocarpa
MPPPQLPPPISRHVAVIGAGAAGLVSARELRREGHDVVVFERDNQVGGTWVYNPRVEPDPLSLDPNRRIIHSSLYSSLRTNLPREVMGFKDYPFIAKNDKKRDQRRFPGHREVLLYLQDFASEFGIEEMVRFDTEVVHVGPVEDNIGKWIVRSKRKISDDDREVSFGFDVDEEIYDAVVICNGHYTEPRIAQIPGISSWPGKQMHSHNYRTPEGFQDQVAILIGSSASSDDISREIAGVAKEVHVASRSVADETYQEQPGYDNMWLHSMIESVHDDGSVIFRNGRVVVADIILHCTGYKYHFPFLDTNGIVTMDENRVAPLYKQVFPPVLAPWLSFVGLPWKVVPFPLVELQTKWIAGVLSGHIALPSPEEMMEDVKAFYETLESSNKPKHYTHNLGGCQFEYDNWLASQCGCPGIEEWRRQMYDAASKSKRLRPEIYRDEWDDDDLVLEAYGDFTKYT

SEQ ID NO: 33 - DNA, 1347 bp, Glycine max
atgatgtccagcgcagtcacactcctgacgccgcgccacgtggcagtgatcggcgcgggcgccgccggcctagtggcggctcgggagctccggcgagaagggcatcgcgtggtggttttcgagaaaggggaggaagtgggtggcatgtgggtgtacagtccggaggtggattcggatccgctgggtttggaggcgaagcggagattagtccactcgagcctctacgattcgctccgaacgaatctgtctcgggagagcatgagtttccgagattaccctttcaggaggagggaggggaaagggagggattctcgaaggttcccgggtcacagagaggtgttactgtacttgcaggatttcgctgctgaatttgaaatcggagaattggtgaggtttggaacggaggttttgtttgctggattggatcagtgtggaaagtggaggctgacttcaacatcaccccatactcatcctgtggatgagatttacgacgcccttatcatttgcaacggccattacgttcagcctcgtcttcctcatatccccgggattaatgcatggccagggaagcagatgcatagccataattatagaacacctgagccctttcaagatcaagttgtagttctaattggtagttctgctagtgcggttgatatctctcgagatatcgcaacagttgctaaagaagtccacattgcagctaggtcagttgaagaagataagctaggaaaggtgcctggccatgagaatatgtggcttcattctatgattgacagcgttcatgaagatggtacagtggtttttcaagatggaaatgcagttggtgctgacttcatcatacattgcacagggtacaagtatgattttcccttccttgaaaccaatggggaggtgactgtagatgacaaccgtgtaggaccactctacaaacatgttttcccaccagccttggctccatggctttcttttgttgggttgccttggaaggttgctcccttctccttgttcgaactgcagagcaagtggatagctggaatcttgtctaatcgcattgcacttccttcgaaagaggagatggctaaagacgttgatgctttttactcatcacttgaagcctctggcactcctaagcgttacactcataatatgggcattcttcagtgggactacaataactggattgcggatcagtgtggggttccttctattgaagaatggagaaggcaaatgtatatagccacatctaagaacagggtgctgcgacccgagtcttaccgtgacgagtgggacgatgatgacttggttctgcaagctcaacaggattttgccaattatctcacttga

SEQ ID NO: 34 - protein, 448 aa, Glycine max
MMSSAVTLLTPRHVAVIGAGAAGLVAARELRREGHRVVVFEKGEEVGGMWVYSPEVDSDPLGLEAKRRLVHSSLYDSLRTNLSRESMSFRDYPFRRREGKGRDSRRFPGHREVLLYLQDFAAEFEIGELVRFGTEVLFAGLDQCGKWRLTSTSPHTHPVDEIYDALIICNGHYVQPRLPHIPGINAWPGKQMHSHNYRTPEPFQDQVVVLIGSSASAVDISRDIATVAKEVHIAARSVEEDKLGKVPGHENMWLHSMIDSVHEDGTVVFQDGNAVGADFIIHCTGYKYDFPFLETNGEVTVDDNRVGPLYKHVFPPALAPWLSFVGLPWKVAPFSLFELQSKWIAGILSNRIALPSKEEMAKDVDAFYSSLEASGTPKRYTHNMGILQWDYNNWIADQCGVPSIEEWRRQMYIATSKNRVLRPESYRDEWDDDDLVLQAQQDFANYLT

SEQ ID NO: 35 - DNA, 1703 bp, Solanum lycopersicum
ggtggcgtgacactcccgctgcctttagtagatccgcccccgctgaatgtggtgaggaatcaaccaacaaatccacatgcaaagatactcaaatacaaaactcaaccaaacaaatccacatgcaaactcaaacacaacataaaatattactttagtcatgattcttaaaaatgtcatcttttcaaaccaaccttatactcgtacacttgtatgccatttgcccatgtctcaaaattcctccaaaaacgtcgccgttattggtgcgggctccgcgggcctcgttgcggcccgagaactccaacgagaaggtcatagagtagttgtattcgaacgagaaaatcaattaggaggcacatgggtttacacgcccgatacagaatccgacccggttgggatcgacccgaatcgggagattgttcattcaagtctttattcatctctccgtgttaatcttccccgggaagtaatgggttttggggattacccgtttgtggccaagaaaaagcccggtagagacccgagaaggtatccgagtcatggggaggtgttggagtatttgaatgattttgctgttgattttgggattattggggttgtgaggtttgggatggaagtggggtttgtgggaaagatggagaatggaaaatggaaggttagttgtagaaagagggaaaatgatgatttgtttgctaatgaggagtatgatgctgttgtaatatgtaatggacactatactgaaccaagaattgctgatattcctggaatcgaagtatggcctggaaagcaaattcacagccacaactaccgtgttcctgacccttttcgagaccaagttgttgtgctgataggtggtgctgcaagtgctactgatatctccagggaaattgctgaagttgctaaagaggtccacatttcttctaggtcagctactagtggagttccgatgaagctgcctggttatgataatatttggctccataatatgattgaagctgttggcagtgatggtggcgtgaattttcaagatgggtcgaaaatccttgctgacatcatcctacactgcacagggtacaaatatcattttcctttcctcgaaactaacgggatagtgactgtggatgacaaccgtgttggtccactttacaagcacgttttcccaccagcctttgcaccaagcctttcatttgttgggctgccttggaaggttataccattcttcttgtgtgaattgcaaagcaagtggatcgctggtgttttatctggtcgaatttctctcccatcaaaggaagatatgaatgctgatattgaagctttctactcatccatggcagcctcttgcattccaaaacggtacactcacaatatggacgactctcagtttgactacgatgattggttggctgctcagtgtggatctacaccctttgaagaatggagaaaacaaatgtacttaatctcaagaaagaacaaaaggactctgcccgagacatatcgtgacgagtgggacgatgatgacttgatcattcaagctcatgaagacttcgtaaaatatattcctgaactagctcaagaacagaagctctcaagatgattaattttgttgttacatgaaaatatagctgaataaatcgagaagtactgtaaataaacaggaaattactcaattaatttcaattgcaactcttgccacatgaaaaaaaaaa

SEQ ID NO: 36 - protein, 477 aa, Solanum lycopersicum
MILKNVIFSNQPYTRTLVCHLPMSQNSSKNVAVIGAGSAGLVAARELQREGHRVVVFERENQLGGTWVYTPDTESDPVGIDPNREIVHSSLYSSLRVNLPREVMGFGDYPFVAKKKPGRDPRRYPSHGEVLEYLNDFAVDFGIIGVVRFGMEVGFVGKMENGKWKVSCRKRENDDLFANEEYDAVVICNGHYTEPRIADIPGIEVWPGKQIHSHNYRVPDPFRDQVVVLIGGAASATDISREIAEVAKEVHISSRSATSGVPMKLPGYDNIWLHNMIEAVGSDGGVNFQDGSKILADIILHCTGYKYHFPFLETNGIVTVDDNRVGPLYKHVFPPAFAPSLSFVGLPWKVIPFFLCELQSKWIAGVLSGRISLPSKEDMNADIEAFYSSMAASCIPKRYTHNMDDSQFDYDDWLAAQCGSTPFEEWRKQMYLISRKNKRTLPETYRDEWDDDDLIIQAHEDFVKYIPELAQEQKLSR

SEQ ID NO: 37 - DNA, 4956 bp, Solanum lycopersicum
atgattcttaaaaatgtcatcttttcaaaccaaccttatactcgtacacttgtatgccatttgcccatgtctcaaaattcctccaaaaacgtcgccgttattggtgcgggctccgcgggcctcgttgcggcccgagaactccaacgagaaggtcatagagtagttgtattcgaacgagaaaatcaattaggaggcacatgggtttacacgcccgatacagaatccgacccggttgggatcgacccgaatcgggagattgttcattcaagtctttattcatctctccgtgttaatcttccccgggaagtaatgggttttggggattacccgtttgtggccaagaaaaagcccggtagagacccgagaaggtatccgagtcatggggaggtgttggagtatttgaatgattttgctgttgattttgggattattggggttgtgaggtttgggatggaagtggggtttgtgggaaagatggagaatggaaaatggaaggttagttgtagaaagagggaaaatgatgatttgtttgctaatgaggagtatgatgctgttgtaatatgtaatggacactatactgaaccaagaattgctgatattcctggtaatttattgaaaaaatttgatctttatgttgaataaagtaccatatctcctttagtcactatcagatttttcgaaattatagattctttggtgaaataagtttaatgctgatgaacttttttacacttctgtgcttttagtaatccagttgtagtaccttttttttagcttttgtgcaccaggtatttgataactttgtcctaggacaaatggaaaaaatgattttaacttgacattttattgacctctagatcatgcccttgggtgcatccaattgtactttgatctttagttgttcatacttgaatagaatattatctccaggagttatgtttctctttgtagtaattgtagattcgtttagttgagggaataagtctaaagtgttgatgaaatttttgttttagtgataacgtttgatatgaagatttcaaacatctgtacattgtgctaatagttacatcctactaactgaaatataagttgtaccctgtgggcgagcaaagttgatctttttatgacaaagatcaaccttcttcttttctctttctgttgttaaggcagtgtccacgagaaagaatgaatattgtacagttaagattagtcttttttcagtggaaagattccagccttctttttggcttagacataattttgtttctcttatgtggcacacatatgttaagtaacttttttcctgttatggttttatacgttgtgagcagttcaaaattttgatttttatttgaattcgcacatcttttccattttctgtcttgttcagaacaaccatataacaaagtgaagtattctgaaattcaatagtgtcttttttaacttgaaagtgtggctgaaattttcttttttccattttaacatcattttgtggcaatactatatgattagctctgagaatatctgtgagttgccaaactattgacatactaagtcaatatgcagtttttgaagtttctaactggaggcctctagcctttcaggaatcgaagtatggcctggaaagcaaattcacagccacaactaccgtgttcctgacccttttcgagaccaagtatgtgtcaggttcattgtttctttaagatgttgatgtttctgaactaatactgttgtgactgaaatgcttgcagttacttcaattatgaatttgctctacttattggaagcgatcttttaattacttactaaaagtgttctgtgatattctattgcgtgaagtttcaggccagatataatagattattgaatggaacgttcttgaacaattctattattgaattcttacttttgcatgaagcactggatgtatgagtgatgacgccaaaaacaaagaaaactttaaattttaaatctaccttttcttcataaatgaatattaatagttagtacaagtatagtagtcctccttcctaggaagctgtttagaaacgatggactatacagaaaatgaacattatgtatatatgcttatatgagcctaggtatgacttgggaaaaatgctccttggttatatcagcttctaatatttgacaaatatagctcaagaaatagtatcatctttttgcctgatcccatggcataactaccaccctttgtaccaagttcagagtaaatgacaaatggttttttgccaggttgttgtgctgataggtggtgctgcaagtgctactgatatctccagggaaattgctgaagttgctaaagaggtccacatttcttctaggtcagctactagtggagttccgatgaagctgcctggttatgataatatttggctccataatatggtaaagtggttaataacttgtatattcatgtggaggtttgtcaacgcttgatgctgaatctgtacttcacatcccctggatactatttaatgaaatcctcttacgtcataaaaagaaaagaaaaaaacacatgatactgaatcgtcagttgcgtaggaaaaaatttatactagctaatgatgcagattgaagctgttggcagtgatggtggcgtgaattttcaagatgggtcgaaaatccttgctgacatcatcctacactgcacagggtgagtgattagtcctattgaaacttcccttttcgcctacggttaaagaaaatgaaagaaagctgacattgatagcctattctttttctttttgaggaaatgtgaagggtaatgcatgcctgtttttctgagggtcataatgatatgaaattgcgaggtgaactgaccttataaaatagcaaaagaatgatgaggtcaacagatagttaaagtaggaactggcatgataagggtagataatggatccaaaggcaagaaacagtaaataaagtagttgatctgctcttgagtaggcagtctcaaaatgaacctcccatattatatgtttaatttcataattgtaagcgtaaaaagatattctaatctataggttagaaacagtaaattaagtagttaatctccttgagtacgccaatctaaaaataaaccttgtagcctttaatacctttaggggctgtttggttgatgggatggcatagacaaggatatcccattggattattttgtcttaccttctacaagggataaaatatcccatcatttatactaaagtggtgggtcaaaataatacctatcaccaatcacaagataaaataaccacatgggatatatcgggattattatcgttatcccatctaccaaatgacccctaagtataagcttaaaggaatgtctattaatattgtcttatcccagacccctaaaagattgaagaatgtctattaatattttcatagcctataagaacatcagaaatccagtagttatctatttaagtattacagtttagtgaggcagatttggttagatattgtgaaggtagccaaagactaaggagtgtaaatttttctgttccgggttacagaaacgaagggaagttatcttcagctaaaatgaatactaatttatgtgagtttggtagtacaggcctcctaattcatagtaagatgtctctaacctttacgattgtaataaaaaataattgctagtaatcttacaaaataatcaaattgatgggagaataagtatcatgaatgtttcttatctttgtttcccaggtacaaatatcattttcctttcctcgaaactaacgggatagtgactgtggatgacaaccgtgttggtccactttacaagcacgttttcccaccagcctttgcaccaagcctttcatttgttgggctgccttggaaggtaaaatttagagggtttgtgctggggtttatagatttacattttaaatggtggacttgaacgtaatatttgtcctggttggcaccaggttataccattcttcttgtgtgaattgcaaagcaagtggatcgctggtgttttatctggtcgaatttctctcccatcaaaggaagatatgaatgctgatattgaagctttctactcatccatggcagcctcttgcattccaaaacggtacactcacaatatggacgactctcaggtataacatctgccaaaaaaccttgtcgaatggttttaggttttttgttttgttctatgggtaaagtgtggtagaaacagctgaacacttcatgcctacccgtaacatacagaagttcttggttacaccatcttagttaagttagaaaagaaaagaaagaaacagagaaaagaatcgaaactgaagaattagagtgtagtaagcttctgtcattaattcagtgcacgatcctgatttagttggggttccaatgtgggcttcaaacaccgggtgggaaacccaaaaagaaaagaaaagaaagaaagcgagtgtagagcttctgtcattgattaacatagactgctaatatgaacacatccaagttgggggttctttagcacggtgacattatagtcgtcctttatgagatatgaatctctgagcgtgttgagcatgtttactttctgtatatgctggctaaacaagattgttgcacagcaacaccaaaccaaatggctttgccccttttatacaagagtgtagctctggtgttcattatgttatgtattatagattaccaaaacagtattacatatagttcgtgcctcgtaaataatccctctctttacagtttgactacgatgattggttggctgctcagtgtggatctacaccctttgaagaatggagaaaacaaatgtacttaatctcaagaaagaacaaaaggactctgcccgagacatatcgtgacgagtgggacgatgatgacttgatcattcaagctcatgaagacttcgtaaaatatattcctgaactagctcaagaacagaagctctcaagatga

SEQ ID NO: 38 - protein, 213 aa, Solanum lycopersicum
MILKNVIFSNQPYTRTLVCHLPMSQNSSKNVAVIGAGSAGLVAARELQREGHRVVVFERENQLGGTWVYTPDTESDPVGIDPNREIVHSSLYSSLRVNLPREVMGFGDYPFVAKKKPGRDPRRYPSHGEVLEYLNDFAVDFGIIGVVRFGMEVGFVGKMENGKWKVSCRKRENDDLFANEEYDAVVICNGHYTEPRIADIPGNLLKKFDLYVE

SEQ ID NO: 39 - DNA, 2044 bp, Homo sapiens
caggacgtagacacacagaagaaaagaagacaaagaacgggttaccatggggaagaaagtggccatcattggagctggtgtgagtggcttggcctccatcaggagctgtctggaagaggggctggagcccacctgctttgagaagagcaatgacattgggggcctgtggaaattttcagaccatgcagaggagggcagggctagcatttacaaatcagtcttttccaactcttccaaagagatgatgtgtttcccagacttcccatttcccgatgacttccccaactttatgcacaacagcaagatccaggaatatatcattgcatttgccaaagaaaagaacctcctgaagtacatacaatttaagacatttgtatccagtgtaaataaacatcctgattttgcaactactggccagtgggatgttaccactgaaagggatggtaaaaaagaatcggctgtctttgatgctgtaatggtttgttccggacatcatgtgtatcccaacctaccaaaagagtcctttccaggactaaaccactttaaaggcaaatgcttccacagcagggactataaagaaccaggtgtattcaatggaaagcgtgtcctggtggttggcctggggaattcgggctgtgatattgccacagaactcagccgcacagcagaacaggtcatgatcagttccagaagtggctcctgggtgatgagccgggtctgggacaatggttatccttgggacatgctgctcgtcactcgatttggaaccttcctcaagaacaatttaccgacagccatctctgactggttgtacatgaagcagatgaatgcaagattcaagcatgaaaactatggcttgatgcctttaaatggagtcctgaggaaagagcctgtatttaacgatgagctcccagcaagcattctgtgtggcattgtgtccgtaaagcctaacgtgaaggaattcacagagacctcggccatttttgaggatgggaccatatttgagggcattgactgtgtaatctttgcaacagggtatagttttgcctaccccttccttgatgagtctatcatcaaaagcagaaacaatgagatcattttatttaaaggagtatttcctcctctacttgagaagtcaaccatagcagtgattggctttgtccagtcccttggggctgccattcccacagttgacctccagtcccgctgggcagcacaagtaataaagggaacttgtactttgccttctatggaagacatgatgaatgatattaatgagaaaatggagaaaaagcgcaaatggtttggcaaaagcgagaccatacagacagattacattgtttatatggatgaactctcctccttcattggggcaaagcccaacatcccatggctgtttctcacagatcccaaattggccatggaagtttattttggcccttgtagtccctaccagtttaggctggtgggcccagggcagtggccaggagccagaaatgccatactgacccagtgggaccggtcgttgaaacccatgcagacacgagtggtcgggagacttcagaagccttgcttctttttccattggctgaagctctttgcaattcctattctgttaatcgctgttttccttgtgttgacctaatcatcattttctctaggatttctgaaagttactgacaatacccagacaggggctttgctatttaaaaattaaaattttcacaccacctgcttttctattcagcatcttttgcagtactctgtagacattagtcagtaatacagtgttatttctaggctctgaaatagccactttaagaatcatgtcatgatcttaagagagcactaatcatttctgtttgagttccactaacacttcaaaatcagaactatgttctttatatctaacttaaatcatttcctgaaacattttgacatgattcctttttccttttaaacaatgtatgaaagatgtattttaaatctaaataaagagcaaattaagcagaataaaaaaaaaaaaaaaaaaaaaaaaaaaaaa

SEQ ID NO: 40 - protein, 532 aa, Homo sapiens
MGKKVAIIGAGVSGLASIRSCLEEGLEPTCFEKSNDIGGLWKFSDHAEEGRASIYKSVFSNSSKEMMCFPDFPFPDDFPNFMHNSKIQEYIIAFAKEKNLLKYIQFKTFVSSVNKHPDFATTGQWDVTTERDGKKESAVFDAVMVCSGHHVYPNLPKESFPGLNHFKGKCFHSRDYKEPGVFNGKRVLVVGLGNSGCDIATELSRTAEQVMISSRSGSWVMSRVWDNGYPWDMLLVTRFGTFLKNNLPTAISDWLYMKQMNARFKHENYGLMPLNGVLRKEPVFNDELPASILCGIVSVKPNVKEFTETSAIFEDGTIFEGIDCVIFATGYSFAYPFLDESIIKSRNNEIILFKGVFPPLLEKSTIAVIGFVQSLGAAIPTVDLQSRWAAQVIKGTCTLPSMEDMMNDINEKMEKKRKWFGKSETIQTDYIVYMDELSSFIGAKPNIPWLFLTDPKLAMEVYFGPCSPYQFRLVGPGQWPGARNAILTQWDRSLKPMQTRVVGRLQKPCFFFHWLKLFAIPILLIAVFLVLT

SEQ ID NO: 41 - DNA, 2190 bp, Oryctolagus cuniculus
tcacagcatccggcggctcgcagcggcgggacatcgcggacgttcgcgcaggcggactagtgacttccacgcaagacaggcgacgctgccgggagaccatggccgggaaaagagtggcggtgattggggcgggagcgagcgggctggcttgcatcaagtgctgcctggaagagggcttggagcccgtctgcttcgaaaggaccgacgacatcggaggtctgtggaggttccaggaaagtcccgacgaaggaagggccagtatctacaaatccgtgatcatcaacacctccaaggagatgatgtgcttcagtgactaccccatcccggatcattttcctaacttcatgcacaactcccaggtcctggagtacttcaggatgtacgccaaagaatttggtcttctgaagtatattcagtttaagaccactgtgtgcagtgtgaagaagcggcctgatttctccacgtcgggccaatgggaggtgctgactgagtgcgaagggaaaaaggagagtgctgtcttcgatggggtcctggtttgcaccggccatcacaccagtgctcacctgccactggaaagcttccctgggattgagaagttcaaagggcagtacttgcacagtcgagactataagaacccagagaaattcactggaaagagagtcattgtcattggcattgggaattctggaggggacctggctgtggagatcagccacacagccaagcaggtcttcctcagcaccaggagaggggcttggatcatgaatcgtgtcggcgaccatggatatcctattgatatactgctgtcttctcgatttagtcaatttttgaagaagattactggtgaaacaatagcaaattcatttttggaaagaaagatgaaccaaaggtttgaccatgcaatgtttggtctgaagcctaaacacagagctttgagtcaacacccaacagtaaatgatgacctgccaaatcgtatcatttctggctctgtcaagatcaaaggaaatgtgaaggaattcacagaaacagctgccatatttgaagatggctccagggaggatgacattgatgctgttatttttgccacaggctatagcttttcctttccttttcttgaagactctgtcaaagtggtgaaaaacaaggtatctctgtataaaaaggtcttcccccctaacctggaaaagccaactcttgcaatcataggcttgatccagcccctgggagccattatgcccatttcagagctccaagcacgatgggccaccctagtgtttaaagggctaaagactttaccctcacaaagtgaaatgatgacagaaatatctcaggttcaagagaaaatggcaaaaaggtatgtggagagccaacgccataccattcagggagactacatagagaccatggaagaaattgctgatttggtgggggtcaggccaaatttgctgtctctggctttcaccgaccccaggctggcattacaattacttttgggaccctgtactccagtccattatcgtctccagggccgtggaaagtgggatggggctcggaaaaccatccttaccgtagaagatcggatcaggaagcctctgatgacaagagtcacggaaagcagtaactctgtgacctcgatgatgacaatgggcaagtttatgctagctattgctttcttagccatagctgtggtttatttttagctgtccctttgtcattgcctctgctttcattgggaagcttaacttagagagagataccttcagaattttacaagatcaaatgaccctcctctttcaaattgccccatttctctttcaaaagcattaattctctcttcattttcctacagtgagatccaagcttttcatttgcactaagcatctcctcacctctcatgagccttcactttctctctccagagcagctcgggtactcttagtcatctttgtatgtccctagcagagtagttgacatttggctggtgtttaaccaatgtttggtgttgtggctcaaagtctgtttttgtatgggaaatgactgactgtataactctgcttgggatggaatttggttttccattatttttgtctttaacattataacaaatgtatgtttcctgagaaataagattaataatgaccttcgtaattgtagacaaataaatacttaagttactttgttctacatgcc

SEQ ID NO: 42 - protein, 533 aa, Oryctolagus cuniculus
MAGKRVAVIGAGASGLACIKCCLEEGLEPVCFERTDDIGGLWRFQESPDEGRASIYKSVIINTSKEMMCFSDYPIPDHFPNFMHNSQVLEYFRMYAKEFGLLKYIQFKTTVCSVKKRPDFSTSGQWEVLTECEGKKESAVFDGVLVCTGHHTSAHLPLESFPGIEKFKGQYLHSRDYKNPEKFTGKRVIVIGIGNSGGDLAVEISHTAKQVFLSTRRGAWIMNRVGDHGYPIDILLSSRFSQFLKKITGETIANSFLERKMNQRFDHAMFGLKPKHRALSQHPTVNDDLPNRIISGSVKIKGNVKEFTETAAIFEDGSREDDIDAVIFATGYSFSFPFLEDSVKVVKNKVSLYKKVFPPNLEKPTLAIIGLIQPLGAIMPISELQARWATLVFKGLKTLPSQSEMMTEISQVQEKMAKRYVESQRHTIQGDYIETMEEIADLVGVRPNLLSLAFTDPRLALQLLLGPCTPVHYRLQGRGKWDGARKTILTVEDRIRKPLMTRVTESSNSVTSMMTMGKFMLAIAFLAIAVVYF

SEQ ID NO: 43 - protein, 445 aa, Artificial Sequence
  Sequence is synthesized
MAPXIXLXTSRXVAVIGAGAAGLVAARELRREGHKVVVFERENQVGGTWVYTPEVESDPLGLDPNRTIVHSSLYXSLRTNLPREVMGFRDYPFVPREGEGRDPRRFPXHREVLXYLEDFAREFGIEELVRFGTEVVFXGLXDGKWRVKSRSEDGDXVXEIFDAVVVCNGHYTEPRVAEIPGIDAWPGKQMHSHNYRTPEPFRDQVVVLIGXSASAVDISRXIAGVAKEVHIASRSVEAETLEKLXGXDNMWLHSMIESVHKDGTVVFQDGSVVLADVILHCTGYKYHFPFLETNGIVTVDDNRVGPLYKHVFPPALAPGLSFVGLPWKVXPFPLFELQSKWIAGVLSGRIALPSXEEMMADVKAFYSSLEASGKPKHYTHNLGDSQXYDNWLAXQCGCPPVEEWRKQMYIATSKNKXARPETYRDEWDDDDLILZAYEDFAKYXX

SEQ ID NO: 44 - DNA, 1397 bp, Artificial Sequence
  Sequence is synthesized
atcatcacacaaaaaagatggcaccagcacgaacccgagtcaactcactcaacgtggcagtgatcggagccggagccgccggactcgtagctgcaagagagctccgccgcgagaatcacaccgtcgtcgttttcgaacgtgactcaaaagtcggaggtctctgggtatacacacctaacagcgaaccagacccgcttagcctcgatccaaaccgaaccatcgtccattcaagcgtctatgattctctccgaaccaatctcccacgagagtgcatgggttacagagacttccccttcgtgcctcgacctgaagatgacgaatcaagagactcgagaaggtaccctagtcacagagaagttcttgcttaccttgaagacttcgctagagaattcaaacttgtggagatggttcgatttaagaccgaagtagttcttgtcgagcctgaagataagaaatggagggttcaatccaaaaattcagatgggatctccaaagatgagatctttgatgctgttgttgtttgtaatggacattatacagaacctagagttgctcatgttcctggtatagattcatggccagggaagcagattcatagccacaattaccgtgttcctgatcaattcaaagaccaggtggtggtagtgataggaaattttgcgagtggagctgatatcagcagggacataacgggagtggctaaagaagtccatatcgcgtctagatcgaatccatctaagacatactcaaaacttcccgggtcaaacaatctatggcttcactctatgatagaaagtgtacacgaagatgggacgattgtttttcagaacggtaaggttgtacaagctgataccattgtgcattgcactggttacaaatatcacttcccatttctcaacaccaatggctatattactgttgaggataactgtgttggaccgctttacgaacatgtctttccgcctgcgcttgctcccgggctttccttcatcggtttaccctggatgacactgcaattctttatgtttgagctccaaagcaagtgggtggctgcagctttgtctggccgggtcacacttccttcagaagagaaaatgatggaagacgttaccgcctactatgcaaagcgtgaggctttcgggcaacctaagagatacacacatcgacttggtggaggtcaggttgattaccttaattggatagcagagcaaattggtgcaccgcccggtgaacaatggagatatcaggaaataaatggcggatactacagacttgctacacaatcagacactttccgtgataagtgggacgatgatcatctcatagttgaggcttatgaggatttcttgagacagaagctgattagtagtcttccttctcagttattggaatcttga

(19) United States
(12) Patent Application Publication
(10) Pub. No.: US 2016/0237450 A1
(43) Pub. Date: Aug. 18, 2016

(54) METHOD FOR ENHANCING DROUGHT TOLERANCE IN PLANTS

(71) Applicants: Plant Response Biotech S.L., Madrid (ES); Consejo Superior de Investigaciones Cientificas, Madrid (ES)

(72) Inventors: Marisé Borja, Madrid (ES); Julio Bonet-Gigante, Madrid (ES); Antonio Molina, Madrid (ES); Rafael Catalá, Madrid (ES); Julio Salinas, Madrid (ES)

(73) Assignees: Plant Response Biotech S.L., Madrid (ES); Consejo Superior de Investigaciones Cientificas, Madrid (ES)

(21) Appl. No.: 14/625,242

(22) Filed: Feb. 18, 2015

(51) Int. Cl. C12N 15/82

(52) U.S. Cl. CPC C12N 15/8273

(57) ABSTRACT

Methods of producing transgenic photosynthetic organisms and plants overexpressing an FMO protein are disclosed. The disclosure also relates to transgenic photosynthetic organisms and plants having between 4 and 37 fold greater expression of an FMO protein compared to wild-type plants, wherein said transgenic photosynthetic organisms and plants have between 1.1 and 3.4 fold greater trimethylamine N-oxide compared to wild-type, and wherein said transgenic photosynthetic organisms plants are drought tolerant. The disclosure further relates to DNA constructs and methods of producing DNA constructs having a promoter operably linked to one or more FMO protein coding sequences. The disclosure further relates to methods of producing drought tolerant plants and photosynthetic organisms by applying an effective amount of trimethylamine N-oxide di-hydrate.

The Sequence Listing associated with this application is filed in electronic format via EFS-Web and is hereby incorporated by reference into the specification in its entirety.

The claimed invention was made by parties to a joint research agreement, within the meaning of 35 U.S.C. 100(h), which was in effect before the effective filing date of the application, and the claimed invention was made as a result of activities undertaken within the scope of the joint research agreement. The parties of the joint research agreement are the State Agency Council for Scientific Research (CSIC), the Institute of National Agricultural Research and Technology and Food (INIA), and Plant Response Biotech, S.L.

### BACKGROUND

When plants are exposed to drought stress conditions brought about by reduced water content in the soil due to a shortage of rainfall or irrigation, physiological functions of cells may deteriorate and thus various disorders may arise in the plant. When subjected to such stress factors, plants may display a variety of mechanistic responses as protective measures, with a resultant adverse effect on growth, development, and productivity. Significant losses in quality and yield are commonly observed.

The foregoing examples of related art and limitations related therewith are intended to be illustrative and not exclusive, and they do not imply any limitations on the inventions described herein. Other limitations of the related art will become apparent to those skilled in the art upon a reading of the specification and a study of the drawings.

### BRIEF DESCRIPTION OF THE SEQUENCE LISTINGS

SEQ ID NO: 1 discloses the At FMO GS-OX5 nucleic acid sequence (NM_101086.4) (At1g12140).

SEQ ID NO: 2 discloses the At FMO GS-OX5 amino acid sequence (NM_101086.4) (At1g12140).

SEQ ID NO: 3 discloses the Br FMO GS-OX1 nucleic acid sequence (FJ376070.1).

SEQ ID NO: 4 discloses the Br FMO GS-OX1 amino acid sequence (FJ376070.1).

SEQ ID NO: 5 discloses the Cs FMO GS-OX3 nucleic acid sequence (XM_004150596.1) (LOC101212991).

SEQ ID NO: 6 discloses the Cs FMO GS-OX3 amino acid sequence (XM_004150596.1) (LOC101212991).

SEQ ID NO: 7 discloses the Cs FMO GS-OX3 nucleic acid sequence (XM_004150602.1) (LOC101220318).

SEQ ID NO: 8 discloses the Cs FMO GS-OX3 amino acid sequence (XM_004150602.1) (LOC101220318).

SEQ ID NO: 9 discloses the Cs FMO GS-OX3 nucleic acid sequence (XM_004170413.1) (LOC101220079).

SEQ ID NO: 10 discloses the Cs FMO GS-OX3 amino acid sequence (XM_004170413.1) (LOC101220079).

SEQ ID NO: 11 discloses the Cs FMO GS-OX3 nucleic acid sequence (XM_004164404.1) (LOC101227975).

SEQ ID NO: 12 discloses the Cs FMO GS-OX3 amino acid sequence (XM_004164404.1) (LOC101227975).

SEQ ID NO: 13 discloses the Mt FMO GS-OX5 nucleic acid sequence (XM_003611223.1) (MTR_5g012130).

SEQ ID NO: 14 discloses the Mt FMO GS-OX5 amino acid sequence (XM_003611223.1) (MTR_5g012130).

SEQ ID NO: 15 discloses the Os FMO nucleic acid sequence (NC_008403.2).

SEQ ID NO: 16 discloses the Os FMO amino acid sequence (NP_001065338.1).

SEQ ID NO: 17 discloses the Vv FMO GS-OX3-3 nucleic acid sequence (XM_003631392.1) (LOC100255688).

SEQ ID NO: 18 discloses the Vv FMO GS-OX3-3 amino acid sequence (XM_003631392.1) (LOC100255688).

SEQ ID NO: 19 discloses the Vv FMO GS-OX3-2 nucleic acid sequence (XM_003631391.1) (LOC100255688).

SEQ ID NO: 20 discloses the Vv FMO GS-OX3-2 amino acid sequence (XM_003631391.1) (LOC100255688).

SEQ ID NO: 21 discloses the Vv FMO GS-OX3-2 nucleic acid sequence (XM_003635084.1) (LOC100242032).

SEQ ID NO: 22 discloses the Vv FMO GS-OX3-2 amino acid sequence (XM_003635084.1) (LOC100242032).

SEQ ID NO: 23 discloses the Gh FMO-1 nucleic acid sequence (DQ122185.1).

SEQ ID NO: 24 discloses the Gh FMO-1 amino acid sequence (DQ122185.1).

SEQ ID NO: 25 discloses the Zm FMO nucleic acid sequence (NM_001157345.1).

SEQ ID NO: 26 discloses the Zm FMO amino acid sequence (NP_001150817.1).

SEQ ID NO: 27 discloses the Pt FMO GS-OX nucleic acid sequence (XM_002329873.1).

SEQ ID NO: 28 discloses the Pt FMO GS-OX amino acid sequence (XM_002329873.1).

SEQ ID NO: 29 discloses the Pt FMO GS-OX nucleic acid sequence (XM_002318967.1).

SEQ ID NO: 30 discloses the Pt FMO GS-OX amino acid sequence (XM_002318967.1).

SEQ ID NO: 31 discloses the Pt FMO GS-OX nucleic acid sequence (XM_002329874.1).

SEQ ID NO: 32 discloses the Pt FMO GS-OX amino acid sequence (XM_002329874.1).

SEQ ID NO: 33 discloses the Gm FMO nucleic acid sequence (NM_003538657.1).

SEQ ID NO: 34 discloses the Gm FMO amino acid sequence (XP_003538705.1).

SEQ ID NO: 35 discloses the Sl FMO GS-OX1 nucleic acid sequence (XM_004241959.1) (LEFL1075CA11).

SEQ ID NO: 36 discloses the Sl FMO GS-OX1 amino acid sequence (XP_004242007.1) (LEFL1075CA11).

SEQ ID NO: 37 discloses the Sl FMO GS-OX1 nucleic acid sequence (SGN-U584070) (Solyc06g060610).

SEQ ID NO: 38 discloses the Sl FMO GS-OX1 amino acid sequence (SGN-U584070) (Solyc06g060610).

SEQ ID NO: 39 discloses the Hs FMO-3 nucleic acid sequence (NC_000001.10 (171,060,018.171, 086,961)).

SEQ ID NO: 40 discloses the Hs FMO-3 amino acid sequence (NP_001002294.1).

SEQ ID NO: 41 discloses the Oc FMO-3 nucleic acid sequence (NC_013681.1).

SEQ ID NO: 42 discloses the Oc FMO-3 amino acid sequence (NP_001075714.1).

SEQ ID NO: 43 discloses the consensus sequence of the polypeptide SEQ ID No. from 2 to 38.

SEQ ID NO: 44 discloses the 5′UTR in combination with the DNA sequence of At FMO GS.

### BRIEF DESCRIPTION OF THE FIGURES

The accompanying figures, which are incorporated herein and form a part of the specification, illustrate some, but not the only or exclusive, example embodiments and/or features. It is intended that the embodiments and figures disclosed herein are to be considered illustrative rather than limiting.

FIG. 1A is a map of a DNA construct that may be used to produce transgenic plants and transgenic photosynthetic organisms for overexpression of a flavin-containing monooxygenase (FMO) protein.

FIG. 1B is a map of a DNA construct that may be used to produce transgenic plants and transgenic photosynthetic organisms for overexpression of two or more FMO proteins.

FIG. 2A is an alternate map of a DNA construct that may be used to produce transgenic plants and transgenic photosynthetic organisms for overexpression of an FMO protein.

FIG. 2B is an alternate map of a DNA construct that may be used to produce transgenic plants and transgenic photosynthetic organisms for overexpression of two or more FMO proteins.

FIG. 3A is a map of an example DNA construct that was used to produce Arabidopsis thaliana plants for constitutive overexpression of the RCI5 FMO protein.

FIG. 3B is a map of an example DNA construct that was used to produce Arabidopsis thaliana plants for stress inducible overexpression the RCI5 FMO protein.

FIG. 4A is a map of an example DNA construct that may be used to obtain Zea mays plants for constitutive overexpression of the Zm FMO protein.

FIG. 4B is a map of an example DNA construct that may be used to obtain Solanum lycopersicum plants for stress inducible overexpression of the Sl FMO GS-OX1 protein coding sequence.

FIG. 5A shows the relative amount of FMO GS-OX5 RNA in wild-type Arabidopsis thaliana and two transgenic lines, designated FMO3X and FMO8X.

FIG. 5B shows the micromolar amount of trimethylamine N-oxide (TMAO) per kilogram of fresh weight in wild-type Arabidopsis thaliana and two transgenic lines, designated FMO3X and FMO8X. As used herein, “fresh weight” means the entire plant, including the roots, stem, shoots, and leaves.

FIG. 6 shows photographs of plants before and after drought recovery. From the bottom, wild type Col-0 (labeled Col-0) Arabidopsis thaliana plants, in the middle (labeled FMO3X), transgenic Arabidopsis thaliana plants overexpressing Arabidopsis thaliana FMO GS-OX5, and in the upper panel (labeled FMO8X) transgenic Arabidopsis thaliana plants overexpressing Arabidopsis thaliana FMO GS-OX5.

FIG. 7 shows overexpression of FMO GS-OX5 activates stress induced gene expression. Bars represent the number of genes whose expression is increased (UP) or decreased (DOWN) in transgenic Arabidopsis plants overexpressing FMO GS-OX5 (RCI5-OE.FMO8X) compared to wild-type plants. It also shows the total number of cold, salt, and drought-inducible genes whose expression is increased in RCI5-OE.FMO8X.

FIG. 8 shows a phylogenetic tree based on protein similarities using the alignment-free algorithm, named CLUSS, for clustering protein families of the polypeptide sequences of FMO from Arabidopsis thaliana, grapevine, Populus trichocarpa, rice, soybean, melon, tomato, sorghum, corn, wheat, barley, human and rabbit.

FIG. 9 shows tomato plants after drought recovery. The plant on the left was irrigated with water and the plant on the right was irrigated with 5.5 g/L TMAO di-hydrate.

FIG. 10 shows the average weight in grams per inflorescence for TMAO di-hydrate constant irrigation of broccoli plants under limited water growing conditions.

FIG. 11 shows the average fresh weight in grams per pepper plant for TMAO di-hydrate spray or TMAO di-hydrate in constant irrigation of treated pepper plants under limited water growing conditions.

FIG. 12 shows the average weight in grams per pepper fruit for TMAO di-hydrate spray or TMAO di-hydrate in constant irrigation of treated pepper plants under limited water growing conditions.

### SUMMARY

The following embodiments and aspects thereof are described and illustrated in conjunction with products and methods, which are meant to be exemplary and illustrative, not limiting in scope. In various embodiments, one or more of the above-described problems have been reduced or eliminated, while other embodiments are directed to other improvements.

One embodiment discloses a method of producing a transgenic photosynthetic organism or plant overexpressing an FMO protein comprising transforming a photosynthetic organism, plant, plant cell, or plant tissue with a sequence encoding a FMO protein operably linked to a promoter, selecting for a photosynthetic organism, plant, plant cell, or plant tissue having said sequence stably integrated into said photosynthetic organism, plant, plant cell, or plant tissue genome, wherein said selecting comprises determining the level of expression of said FMO protein and selecting a photosynthetic organism having between 4 and 37 fold greater expression of said FMO protein compared to wild type, and producing a transgenic photosynthetic organism or plant overexpressing an FMO protein.

Another embodiment discloses a DNA construct comprising a promoter operably linked to a marker, and a promoter operably linked to one or more FMO protein coding sequences, wherein said promoter operably linked to one or more FMO protein coding sequences is selected from the group consisting of 35S, Pro_{RD29A}, and Ubiquitin, and wherein said one or more FMO protein coding sequences has between 90% and 100% identity to the sequence as shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41 or SEQ ID NO: 44.

As used herein, “marker” means any selectable marker or reporter gene.

Another embodiment discloses a drought tolerant transgenic plant having one or more DNA constructs stably integrated into said plants genome, wherein said DNA construct comprises an FMO protein coding sequence operably linked to a promoter, wherein said plant overexpresses said FMO protein between 4 and 37 fold greater than the level of FMO expression in non-transgenic plants, wherein said overexpression of said FMO protein catalyzes the oxidation of endogenous metabolites containing nucleophilic nitrogen, and wherein said transgenic plant has between 1.1 and 3.4 fold greater trimethylamine N-oxide.

Another embodiment discloses a method for producing a drought tolerant plant or photosynthetic organism comprising applying an effective amount of trimethylamine N-oxide di-hydrate to a plant, plant part, photosynthetic organism or seed, and growing the plant, plant part, photosynthetic organism or seed, wherein a drought tolerant plant or photosynthetic organism is produced.

Another embodiment discloses a drought tolerant plant or photosynthetic organism produced from applying an effective amount of TMAO di-hydrate to a plant, plant part, photosynthetic organism or a seed and growing the plant, plant part, photosynthetic organism or seed.

Another embodiment discloses a method for increasing the endogenous level of trimethylamine N-oxide in a plant or photosynthetic organism comprising applying an effective amount of trimethylamine N-oxide di-hydrate to produce a plant or photosynthetic organism having between 1.1 and 9.9 fold greater endogenous TMAO compared to a plant or photosynthetic organism that has not been treated with TMAO di-hydrate.

In addition to the exemplary aspects and embodiments described above, further aspects and embodiments will become apparent by study of the following descriptions.

### DETAILED DESCRIPTION

Embodiments include methods of producing a transgenic plant or transgenic photosynthetic organism overexpressing an FMO protein, wherein the method comprises transforming a plant, plant cell, plant tissue, or photosynthetic organism with a sequence encoding an FMO protein operably linked to a promoter, selecting for a plant, plant cell, plant tissue, or photosynthetic organism having said sequence stably integrated into said plant, plant cell, plant tissue, or photosynthetic organisms genome, wherein said selecting comprises determining the level of expression of said FMO protein and selecting a plant, plant cell, plant tissue, or photosynthetic organism having between 4 and 37 fold greater expression of said FMO protein compared to wild type, and producing a transgenic plant or transgenic photosynthetic organism overexpressing an FMO protein.

As used herein, “fold greater” or “fold increase” means the amount multiplied over the starting value. For example, if the starting value is 100, a 1.1 fold increase would yield a value of 110; a 1.2 fold increase would yield a value of 120, and likewise a 3.5 fold increase would yield a value of 350.

As used herein, “plants” means all monocotyledonous and dicotyledonous plants, and all annual and perennial dicotyledonous and monocotyledonous plants included by way of example, but not by limitation, to those of the genera Glycine, Vitis, Asparagus, Populus, Pennisetum, Lolium, Oryza, Zea, Avena, Hordeum, Secale, Triticum, Sorghum, Saccharum and Lycopersicum, and the class Liliatae. “Plants” also includes mature plants, seeds, shoots and seedlings, plant parts, propagation material, plant organs, tissue, protoplasts, callus and other cultures, for example cell cultures derived from the above, and all other types of associations of plant cells which give functional or structural units. “Mature plants” means plants at any developmental stage beyond the seedling stage. “Seedling” means a young, immature plant in an early developmental stage.

As used herein the term “photosynthetic organisms” may include, but is not limited to, organisms such as Arthrospira spp., Spirulina spp., Synechococcus elongatus, Synechococcus spp., Synechosystis spp., Synechosystis spp., Spirulina plantensis, Calothrix spp., Anabaena flos-aquae, Aphanizomenon spp., Anabaena spp., Gleotrichia spp., Oscillatoria spp. and Nostoc spp.; eukaryotic unicellular algae such as but not limited to Chaetoceros spp., Chlamydomonas reinhardtii, Chlamydomonas spp., Chlorella vulgaris, Chlorella spp., Cyclotella spp., Didymosphenia spp., Dunaliella tertiolecta, Dunaliella spp., Botryococcus braunii, Botryococcus spp., Gelidium spp., Gracilaria spp., Hantzschia spp., Hematococcus spp., Isochrysis spp., Laminaria spp., Nannochloropsis spp., Navicula spp., Nereocystis luetkeana, Pleurochrysis spp., Postelsia palmaeformis, and Sargassum spp.

As used herein, “transgenic plant’ and “transgenic photosynthetic organism” relates to plants and photosynthetic organisms which have been genetically modified to contain DNA constructs, as will be discussed further herein.

A variety of seeds or bulbs may be used in the methods described herein including but are not limited to plants in the families' Solanaceae and Cucurbitaceae, as well as plants selected from the plant genera Calibrachoa, Capsicum, Nicotiana, Nierembergia, Petunia, Solanum, Cucurbita, Cucumis, Citrullus, Glycine, such as Glycine max (Soy), Calibrachoa x hybrida, Capsicum annuum (pepper), Nicotiana tabacum (tobacco), Nierenbergia scoparia (cupflower), Petunia, Solanumlycopersicum (tomato), Solanum tuberosum (potato), Solanum melongena (eggplant), Cucurbita maxima (squash), Cucurbita pepo (pumpkin, zucchini), Cucumis metuliferus (Horned melon) Cucumis melo (Musk melon), Cucumis sativus (cucumber) and Citrullus lanatus (watermelon). Various monocotyledonous plants, in particular those which belong to the family Poaceae, may be used with the methods described herein, including but not limited to, plants selected from the plant genera Hordeum, Avena, Secale, Triticum, Sorghum, Zea, Saccharum, Oryza, Hordeum vulgare (barley), Triticum aestivum (wheat), Triticum aestivum subsp. spelta (spelt), Triticale, Avena sativa (oats), Secale cereale (rye), Sorghum bicolor (sorghum), Zea mays (maize), Saccharum officinarum (sugarcane) and Oryza sativa (rice).

Additional examples of plants in which drought tolerance may be produced using the methods described herein include the following crops: rice, corn, canola, soybean, wheat, buckwheat, beet, rapeseed, sunflower, sugar cane, tobacco, and pea, etc.; vegetables: solanaceous vegetables such as paprika and potato; cucurbitaceous vegetables; cruciferous vegetables such as Japanese radish, white turnip, horseradish, kohlrabi, Chinese cabbage, cabbage, leaf mustard, broccoli, and cauliflower, asteraceous vegetables such as burdock, crown daisy, artichoke, and lettuce; liliaceous vegetables such as green onion, onion, garlic, and asparagus; ammiaceous vegetables such as carrot, parsley, celery, and parsnip; chenopodiaceous vegetables such as spinach, Swiss chard; lamiaceous vegetables such as Perilla frutescens, mint, basil; strawberry, sweet potato, Dioscorea japonica, colocasia; flowers; foliage plants; grasses; fruits: pomaceous fruits (apple, pear, Japanese pear, Chinese quince, quince, etc.), stone fleshy fruits (peach, plum, nectarine, Prunus mume, cherry fruit, apricot, prune, etc.), citrus fruits (Citrus unshiu, orange, tangerine, lemon, lime, grapefruit, etc.), nuts (chestnuts, walnuts, hazelnuts, almond, pistachio, cashew nuts, macadamia nuts, etc.), berries (blueberry, cranberry, blackberry, raspberry, etc.), grape, kaki fruit, olive, Japanese plum, banana, coffee, date palm, coconuts, etc.; and trees other than fruit trees; tea, mulberry, flowering plant, roadside trees (ash, birch, dogwood, Eucalyptus, Ginkgo biloba, lilac, maple, Quercus, poplar, Judas tree, Liquidambar formosana, plane tree, zelkova, Japanese arborvitae, fir wood, hemlock, juniper, Pinus, Picea, and Taxus cuspidata).

An embodiment of the present disclosure further provides for transgenic photosynthetic organisms or plants having between 4.1 and 9.9 fold greater expression of an FMO protein compared to non-transformed plants and photosynthetic organisms.

An embodiment of the present disclosure further provides for transgenic photosynthetic organisms or plants having between 10 and 16.9 fold greater expression of an FMO protein compared to non-transformed plants and photosynthetic organisms.

An embodiment of the present disclosure further provides for transgenic photosynthetic organisms or plants having between 17 and 24.9 fold greater expression of an FMO protein compared to non-transformed plants and photosynthetic organisms.

An embodiment of the present disclosure further provides for transgenic photosynthetic organisms or plants having between 25 and 36.9 fold greater expression of an FMO protein compared to non-transformed plants and photosynthetic organisms.

### Gene Expression Analysis

There are a number of methods known in the art to examine the expression level of genes. For example, a northern blot is a technique wherein RNA samples are separated by size via electrophoresis and then specific sequences are detected with a hybridization probe. A northern blot enables the detection of and relative abundance of a particular RNA. Reverse transcriptase-PCR and Real-Time PCR also test for the presence of a particular RNA and enable quantification of gene expression. RNA-seq (RNA Sequencing), also called Whole Transcriptome Shotgun Sequencing, is a technology that uses the capabilities of next-generation sequencing to reveal a snapshot of RNA presence and quantity from a genome at a given moment in time; it is a technique that sequences the entire RNA transcriptome of an organism which also enables quantification of gene expression.

An embodiment of the present disclosure further provides methods of producing a transgenic plant or transgenic photosynthetic organism overexpressing an FMO protein, wherein said FMO protein catalyzes the oxidation of endogenous metabolites containing nucleophilic nitrogen.

An embodiment of the present disclosure further provides for DNA constructs comprising a promoter operably linked to a marker, and a promoter operably linked to one or more FMO protein coding sequences.

### Promoters

A promoter is a DNA region which includes sequences sufficient to cause transcription of an associated (downstream) sequence. A variety of promoters may be used in the methods described herein. Many suitable promoters for use in plants or photosynthetic organisms are well known in the art. The promoter may be regulated, for example, by a specific tissue or inducible by a stress, pathogen, wound, or chemical. It may be naturally-occurring, may be composed of portions of various naturally occurring promoters, or may be partially or totally synthetic. Also, the location of the promoter relative to the transcription start may be optimized.

The promoters can be selected based on the desired outcome. That is, the nucleic acids can be combined with constitutive, tissue-preferred, or other promoters for expression in the host cell of interest. The promoter may be inducible or constitutive.

### Constitutive Promoters

In another embodiment, the overexpression of the FMO protein coding sequences is driven by a constitutive promoter for constitutive overexpression of an FMO protein.

As used herein a “constitutive” promoter means those promoters which enable overexpression in numerous tissues over a relatively large period of a plants or photosynthetic organism's development. For example, a plant promoter or a promoter derived from a plant virus with the methods described herein including but not limited to the 35S transcript of the CaMV cauliflower mosaic virus (Franck et al. Cell 21, 285 (1980); Odell et al. Nature 313, 810 (1985); Shewmaker et al. Virology 140, 281 (1985); Gardner et al. Plant Mol Biol 6, 221 (1986)) or the 19S CaMV Promoter (U.S. Pat. No. 5,352,606; WO 84/02913; Benfey et al. EMBO J. 8, 2195-2202 (1989)). A further suitable constitutive promoter is the rubisco small subunit (SSU) promoter (U.S. Pat. No. 4,962,028), the promoter of Agrobacterium nopaline synthase, the TR double promoter, the Agrobacterium OCS (octopine synthase) promoter, the ubiquitin promoter (Holtorf S et al. Plant Mol Biol 29, 637 (1995)), the ubiquitin 1 promoter (Christensen et al. Plant Mol Biol 18, 675 (1992); Bruce et al. Proc Natl Acad Sci USA 86, 9692 (1989)), the Smas promoter, the cinnamyl-alcohol dehydrogenase promoter (U.S. Pat. No. 5,683,439), the promoters of vacuolar ATPase subunits or the promoter of a proline-rich protein from wheat (WO 91/13991), and further promoters of genes whose constitutive expression in plants is known to the skilled worker including the promoter of nitrilase-1 (nit1) gene from A. thaliana (GenBank Acc. No.: Y07648.2, Nucleotide 2456-4340, Hillebrand et al. Gene 170, 197 (1996)).

### Stress Induced Promoters

In another embodiment, the overexpression of the FMO protein coding sequences is driven by a stress-inducible promoter.

Stress induced promoters (for example RD29 (Singh et al. Plant Cell Rep 30:1019-1028 (2011)) may be selected from the group consisting of a promoter induced by: osmotic stress, drought stress, cold stress, heat stress, oxidative stress, nutrient deficiency, infection by a fungus, infection by an oomycete, infection by a virus, infection by a bacterium, nematode infestation, pest infestation, weed infestation, and herbivory.

Other promoters are those which are induced by biotic or abiotic stress, such as, for example, the pathogen-inducible promoter of the PRP1 gene (or gst1 promoter) from potato (WO 96128561; Ward et al. Plant Mol Biol 22, 361 (1993)), the heat-inducible hsp70 or hsp80 promoter from tomato (U.S. Pat. No. 5,187,267), the chill-inducible alpha-amylase promoter from potato (WO 96/12814) and the light-inducible PPDK promoter or the wounding-inducible pinII promoter (EP-A 0 375 091).

In another embodiment, the overexpression of the FMO protein coding sequence is driven by a drought stress inducible promoter. As used herein the term “drought stress” means plants under conditions where reduced water content in the soil, due to a shortage of rainfall or irrigation, leads to impaired or reduced water absorption by the plant or photosynthetic organism. Drought stress in plants may trigger a deterioration of physiological functions of cells, thereby leading to various disorders. While the conditions which induce drought stress may vary depending on the kind of the soil where the plants are cultivated, examples of the conditions include but are not limited to: a reduction in the water content in the soil of 7.5% by weight or less, more severely 10% by weight or less, and still more severely 15% by weight or less; or the pF value of the soil of 2.3 or more, more severely of 2.7 or more, and still more severely of 3.0 or more.

### Seed Specific Promoters

Seed-specific promoters may also be used. For example, the promoter of phaseolin (U.S. Pat. No. 5,504,200; Bustos et al. Plant Cell 1(9), 839 (1989)), of the 2S albumin gene (Joseffson et al. J Biol Chem 262, 12196 (1987)), of legumin (Shirsat et al. Mol Gen Genet 215(2), 326 (1989)), of the USP (unknown seed protein; Bäumlein et al. Mol Gen Genet 225(3), 459 (1991)), of the napin gene (U.S. Pat. No. 5,608,152; Stalberg et al. L Planta 199, 515 (1996)), of the gene coding for the sucrose binding protein (WO00/26388), the legumin B4 promoter (LeB4; Bäumlein et al. Mol Gen Genet 225, 121 (1991); Bäumlein et al. Plant Journal 2(2), 233 (1992); Fiedler et al. Biotechnology (NY) 13(10), 1090 (1995)), the oleosin promoter from Arabidopsis (WO 98/45461), or the Bce4 promoter from Brassica (WO 91/13980). Further suitable seed specific promoters are those of the glutenin gene (HMWG), gliadin gene, branching enzyme, ADP glucose pyrophosphatase (AGPase) or starch synthase. Further promoters may include those allowing seed specific expression in monocotyledons such as maize, barley, wheat, rye, rice, etc. It is also possible to employ the promoter of the Ipt2 or Ipt1 gene (WO 95/15389, WO 95/23230) or the promoters described in WO 99/16890 (promoters of the hordein gene, of the oryzin gene, of the prolamin gene, of the zein gene, of the kasirin gene or of the secalin gene).

### Tissue Specific Promoters

In another embodiment, the overexpression of the FMO protein coding sequences is driven by a tissue specific promoter, such as those controlling expression in tuber, storage root, or root specific promoters may also be utilized. For example, the patatin class I promoter (B33) or the promoter of the potato cathepsin D inhibitor. Leaf-specific promoters, for example, the promoter of the cytosolic FBPase from potato (WO 97/05900), the SSU promoter (small subunit) of the rubisco (ribulose-1.5-bisphosphate carboxylase) or the ST-LSI promoter from potato (Stockhaus et al. EMBO J. 8, 2445 (1989)).

Epidermis-specific promoters, for example the promoter of the OXLP gene (“oxalate oxidase like protein”; Wei et al. Plant Mol. Biol. 36, 101 (1998)) and a promoter consisting of the GSTA1 promoter and the WIR1a intron (WO 2005/035766) and the GLP4 promoter (WO 2006/1288832 PCT/EP 2006/062747, acc. AJ310534 (Wei, Plant Molecular Biology 36, 101 (1998)). Additional examples of epidermis-specific promoters are, WIR5 (=GstA1), acc. X56012 (Dudler & Schweizer, unpublished); GLP2a, acc. AJ237942 (Schweizer, Plant J. 20, 541 (1999).); Prx7, acc. AJ003141 (Kristensen, Molecular Plant Pathology 2 (6), 311 (2001)); GerA, acc. AF250933 (Wu, Plant Phys. Biochem. 38 or 685 (2000)); OsROC1, acc. AP004656; RTBV, acc. AAV62708, AAV62707 (Klöti, PMB 40, 249(1999)) and Cer3 (Hannoufa, Plant J. 10 (3), 459 (1996)).

In another embodiment, the methods described herein employ mesophyll-tissue-specific promoters such as, for example, the promoter of the wheat germin 9f-3.8 gene (GenBank Acc. No.: M63224) or the barley GerA promoter (WO 02/057412). The promoters are both mesophyll-tissue-specific and pathogen-inducible. Also suitable is the mesophyll-tissue-specific Arabidopsis CAB-2 promoter (GenBank Acc. No.: X15222), and the Zea mays PPCZm1 promoter (GenBank Acc.-No.: X63869) or homologs thereof.

Additional mesophyll-specific promoters include PPCZm1 (=PEPC; Kausch, Plant Mol. Biol. 45, 1 (2001)); OsrbcS (Kyozuka et al., Plant Phys. 102, 991-(1993)); OsPPDK, acc. AC099041; TaGF-2.8, acc. M63223 (Schweizer, Plant J. 20, 541 (1999)); TaFBPase, acc. X53957; TaWIS1, acc. AF467542 (US 20021115849); HvBIS1, acc. AF467539 (US 2002/115849); ZmMIS1, acc. AF467514 (US 2002/115849); HvPR1a, acc. X74939 (Bryngelsson et al., Molecular Plant-Microbe Interactions 7 (2), 267 (1994); HvPR1b, acc. X74940 (Bryngelsson et al., Molecular Plant-Microbe Interactions 7 (2), 267 (1994)); HvB1.3gluc; acc. AF479647; HvPrx8, acc. AJ276227 (Kristensen et al., Molecular Plant Pathology 2 (6), 311 (2001)); and HvPAL, acc. X97313 (Wei, Plant Molecular Biology 36, 101 (1998)).

Examples of other tissue specific promoters are: flower specific promoters, for example the phytoene synthase promoter (WO 92/16635) or the promoter of the Prr gene (WO 98/22593) and anther specific promoters, for example the 5126 promoter (U.S. Pat. Nos. 5,689,049 and 5,689,051), the glob-I promoter and the [gamma]-zein promoter.

Moreover, a person having ordinary skill in the art is capable of isolating further tissue specific suitable promoters by means of routine methods. Thus, the person skilled in the art can identify for example further epidermis-specific regulatory nucleic acid elements, with the aid of customary methods of molecular biology, for example with hybridization experiments or with DNA-protein binding studies. Here, a first step involves, for example, the isolation of the desired tissue from the desired organism from which the regulatory sequences are to be isolated, wherefrom the total poly(A)+RNA is isolated and a cDNA library is established. In a second step, those clones from the first library whose corresponding poly(A)+RNA molecules only accumulate in the desired tissue are identified by means of hybridization with the aid of cDNA clones which are based on poly(A)+RNA molecules from another tissue. Then, promoters with tissue-specific regulatory elements are isolated with the aid of these cDNAs thus identified. Moreover, a person skilled in the art has available further PCR-based methods for the isolation of suitable tissue-specific promoters.

### Chemically Inducible Promoters

Chemically inducible promoters (review article: Gatz et al. Annu. Rev. Plant Physiol Plant Mol Biol 48, 89 (1997)) through which expression of the exogenous gene in the plant can be controlled at a particular point in time may also be utilized. For example, the PRP1 promoter (Ward et al. Plant Mol Biol 22, 361 (1993)), a salicylic acid-inducible promoter (WO 95/19443), a benzenesulfonamide-inducible promoter (EP 0 388 186), a tetracycline-inducible promoter (Gatz et al. Plant J 2, 397 (1992)), an abscisic acid-inducible promoter (EP 0 335 528) and an ethanol- or cyclohexanone-inducible promoter (WO 93/21334) can likewise be used.

### Pathogen Inducible Promoters

Pathogen-inducible promoters may also be utilized, which make possible expression of a gene when the plant is attacked by pathogens. Pathogen-inducible promoters comprise the promoters of genes which are induced as a result of pathogen attack, such as, for example, genes of PR proteins, SAR proteins, [beta]-1.3-glucanase, chitinase, etc. (for example Redolfi et al. Neth J Plant Pathol 89, 245 (1983); Uknes, et al. Plant Cell 4, 645 (1992); Van Loon Plant Mol Viral 4, 111 (1985); Marineau et al. Plant Mol Bid 9, 335 (1987); Matton et al. Molecular Plant-Microbe Interactions 2, 325 (1987); Somssich et al. Proc Natl Acad Sci USA 83, 2427 (1986); Somssich et al. Mol Gen Genetics 2, 93 (1988); Chen et al. Plant J 10, 955 (1996); Zhang and Sing Proc Natl Acad Sci USA 91, 2507 (1994); Warner, et al. Plant J 3, 191 (1993); Siebertz et al. Plant Cell 1, 961 (1989)).

A source of further pathogen-inducible promoters may include the pathogenesis-related (PR) gene family. The nucleotide region of nucleotide −364 to nucleotide −288 in the promoter of PR-2d mediates salicylate specificity (Buchel et al. Plant Mol Biol 30, 493 (1996)). In tobacco, this region binds a nuclear protein whose abundance is increased by salicylate. The PR-1 promoters from tobacco and Arabidopsis (EP-A 0 332 104, WO 98/03536) are also suitable as pathogen-inducible promoters. Also useful, since particularly specifically induced by pathogens, are the “acidic PR-5”-(aPR5) promoters from barley (Schweizer et al. Plant Physiol 114, 79 (1997)) and wheat (Rebmann et al. Plant Mol Biol 16, 329 (1991)). aPR5 proteins accumulate within approximately 4 to 6 hours after attack by pathogens and only show very little background expression (WO 99/66057). One approach for obtaining an increased pathogen-induced specificity is the generation of synthetic promoters from combinations of known pathogen-responsive elements (Rushton et al. Plant Cell 14, 749 (2002); WO 00/01830; WO 99/66057).

Further pathogen-inducible promoters comprise the Flachs Fis1 promoter (WO 96/34949), the Vst1 promoter (Schubert et al. Plant Mol Biol 34, 417 (1997)) and the tobacco EAS4 sesquiterpene cyclase promoter (U.S. Pat. No. 6,100,451). Other pathogen-inducible promoters from different species are known to the skilled worker (EP-A 1 165 794; EP-A 1 062 356; EP-A 1 041 148; EP-A 1 032 684).

### Wounding Inducible Promoters

An additional promoter for the overexpression of an FMO protein as described herein may include wounding-inducible promoters such as that of the pinII gene (Ryan Ann Rev Phytopath 28, 425 (1990); Duan et al. Nat Biotech 14, 494 (1996)), of the wun1 and wun2 gene (U.S. Pat. No. 5,428,148), of the win1 and win2 gene (Stanford et al. Mol Gen Genet 215, 200 (1989)), of the systemin gene (McGurl et al. Science 225, 1570 (1992)), of the WIP1 gene (Rohmeier et al. Plant Mol Biol 22, 783 (1993); Eckelkamp et al. FEBS Letters 323, 73 (1993)), of the MPI gene (Corderok et al. Plant J 6(2), 141 (1994)) and the like.

Examples of additional promoters suitable for the expression of FMO proteins include fruit ripening-specific promoters such as, for example, the fruit ripening-specific promoter from tomato (WO 94/21794, EP 409 625). Development-dependent promoters include some of the tissue-specific promoters because the development of individual tissues naturally takes place in a development-dependent manner.

Constitutive, and leaf and/or stem-specific, pathogen-inducible, root-specific, mesophyll-tissue-specific promoters may be used in conjunction with constitutive, pathogen-inducible, mesophyll-tissue-specific and root-specific promoters. A further possibility for promoters which make expression possible in additional plant tissues or in other organisms such as, for example, E. coli bacteria, to be operably linked to the nucleic acid sequence to be expressed or overexpressed. All the promoters described above are in principle suitable as plant or photosynthetic organism promoters. Other promoters which are suitable for expression in plants are described (Rogers et al. Meth in Enzymol 153, 253 (1987); Schardl et al. Gene 61, 1 (1987); Berger et al. Proc Natl Acad Sci USA 86, 8402 (1989)).

The nucleic acid sequences present in the DNA constructs described herein may be operably linked to additional genetic control sequences. The term genetic control sequences has a wide meaning and means all sequences which have an influence on the synthesis or the function of the recombinant nucleic acid molecule of the invention. For example, genetic control sequences can modify transcription and translation in prokaryotic or eukaryotic organisms.

The DNA constructs may further comprise a promoter with an abovementioned specificity 5′-upstream from the particular nucleic acid sequence which is to be expressed transgenically, and a terminator sequence as additional genetic control sequence 3′-downstream, and if appropriate further conventional regulatory elements, in each case operably linked to the nucleic acid sequence to be expressed.

Genetic control sequences also comprise further promoters, promoter elements or minimal promoters capable of modifying the expression-controlling properties. It is thus possible, for example through genetic control sequences, for tissue-specific expression to take place additionally dependent on particular stress factors. Corresponding elements are described, for example, for drought stress, abscisic acid (Lam E and Chua N H, J Biol Chem 266(26): 17131 (1991)) and heat stress (Schoffl. F et al., Molecular & General Genetics 217(2-3): 246, 1989).

Genetic control sequences further comprise also the 5′-untranslated regions (5′-UTR), introns or noncoding 3′ region of genes such as, for example, the actin-1 intron, or the Adh1-S introns 1, 2 and 6 (generally: The Maize Handbook, Chapter 116, Freeling and Walbot, Eds., Springer, New York (1994)). It has been shown that these may play a significant function in the regulation of gene expression. It has thus been shown that 5′-untranslated sequences are capable of enhancing transient expression of heterologous genes. An example of a translation enhancer which may be mentioned is the 5′ leader sequence from the tobacco mosaic virus (Gallie et al. Nucl Acids Res 15, 8693 (1987)) and the like. They may in addition promote tissue specificity (Rouster J et al. Plant J 15, 435 (1998)), for example, the natural 5′-UTR of the At FMO GS-OX5 or Zm FMO gene.

The FMO family of proteins are present in a wide range of species, including but not limited to, rabbit, human, barley, wheat, corn, sorghum, tomato, melon, soybean, rice, grapevine, broadleaf trees, and species of the Brassicaceae family. By way of example, human FMO1 and FMO3 proteins have an identity of 53% and 84% with the FMO3 proteins from rabbit (see Lawton et al, 1994, Archives of Biochemistry and Biophysics, Vol. 308, 254-257).

“FMO protein” is understood as meaning a sequence which comprises an N-terminal domain, a flavin-monooxygenase domain and a C-terminal domain (Li et al., Plant Physiol. 148(3):1721-33 (2008). FMO proteins can increases endogenous TMAO levels via catalyzing the conversion of trimethylamine (TMA) to trimethylamine N-oxide (TMAO) in the presence of FAD and NADPH. The activity can be determined in an in vitro assay as shown, for instance, in example 2.2 of PCT application WO20100348262.

In another embodiment, the one or more FMO protein coding sequences comprises an amino acid sequence selected from SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO:16, SEQ ID NO:18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID, NO: 38 SEQ ID NO: 40, SEQ ID NO: 42 and SEQ ID NO: 43, and sequences coding for a functionally equivalent variant of the above sequences having between 40% and 49.99% identity, between 50% and 59.99% identity, between 60% and 69.99% identity, between 70% and 79.99% identity, between 80% and 89.99% identity, between 90% and 95.99% identity, and between 96% and 99.99% identity.

In another embodiment, the one or more FMO protein coding sequences comprises a nucleic acid sequence selected from SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41 or SEQ ID NO: 44, and sequences coding for a functionally equivalent variant of the above sequences having between 40% and 49.99% identity, between 50% and 59.99% identity, between 60% and 69.99% identity, between 70% and 79.99% identity, between 80% and 89.99% identity, between 90% and 95.99% identity, and between 96% and 99.99% identity.

The term “Functionally equivalent variant” as used herein means all those FMO sequence variants and proteins derived therefrom wherein the function is substantially maintained, particularly the ability to catalyze the conversion of TMA to TMAO. It is well known in the art that the genetic code is degenerate, meaning that more than one codon may code for the same amino acid. Indeed, all amino acids, with the exception of methionine and tryptophan, have at least two codons that code for them. For example, phenylalanine is coded for by codons UUU and UUC. Likewise, AAA and AAG both code for lysine. Serine, proline, threonine, alanine, valine, and glycine each have four different codons that code for them. Leucine and arginine are each coded for by 6 different codons. Thus, a genetic sequence may be manipulated by mutagenesis, or by natural evolution, to contain different nucleotides while still coding for the same amino acid sequence.

Further, many amino acids have similar structures and chemical properties. Therefore, one can exchange one amino acid for another having a similar structure and chemical property without disrupting the structure or function of the protein, thus creating a functionally equivalent variant.

### Mutation

As used herein the “modification” of nucleotide sequences or amino acid sequences comprises mutating them, or mutations. For the purposes described here, “mutations” means the modification of the nucleic acid sequence of a gene variant in a plasmid or in the genome of an organism. Mutations can be generated, for example as the consequence of errors during replication, or by mutagens. The spontaneous mutation rate in the cell genome of organisms is very low; however, the skilled person in the art knows a multiplicity of biological, chemical and physical mutagens and methods of mutating nucleotide sequences in a random or targeted manner, and therefore ultimately potentially also for modifying the amino acid sequences which they encode.

Mutations comprise substitutions, additions, and deletions of one or more nucleic acid residues. Substitutions are understood as meaning the exchange of individual nucleic acid bases, where one distinguishes between transitions (substitution of a purine base for a purine base, and of a pyrimidine base for a pyrimidine base) and transversions (substitution of a purine base for a pyrimidine base, or vice versa).

Addition or insertion is understood as meaning the incorporation of additional nucleic acid residues in the DNA, which may result in reading-frame shifts. In the case of such reading frame shifts, one distinguishes between in-frame insertions/additions and out-of-frame insertions. In the case of the in-frame insertions/additions, the reading frame is retained, and a polypeptide which is lengthened by the number of the amino acids encoded by the inserted nucleic acids is formed. In the case of out-of-frame insertions/additions, the original reading frame is lost, and the formation of a complete and functional polypeptide is in many cases no longer possible, which of course depends on the site of the mutation.

Deletions describe the loss of one or more base pairs, which likewise leads to in-frame or out-of-frame reading-frame shifts and the consequences which this entails with regard to the formation of an intact protein.

One skilled in the art would be familiar with the mutagenic agents (mutagens) which can be used for generating random or targeted mutations and both the methods and techniques which may be employed. Such methods and mutagens are described for example in van Harten A. M. (“Mutation breeding: theory and practical applications”, Cambridge University Press, Cambridge, UK (1998)), Friedberg E., Walker G., Siede W. (“DNA Repair and Mutagenesis”, Blackwell Publishing (1995)), or Sankaranarayanan K., Gentile J. M., Ferguson L. R. (“Protocols in Mutagenesis”, Elsevier Health Sciences (2000)).

Customary methods and processes of molecular biology such as, for example, the in vitro mutagenesis kit, “LA PCR in vitro Mutagenesis Kit” (Takara Shuzo, Kyoto), or PCR mutagenesis using suitable primers, may be employed for introducing targeted mutations. As mentioned herein, a multiplicity of chemical, physical and biological mutagens exists. Those mentioned herein below are given by way of example, but not by limitation.

Chemical mutagens may be divided according to their mechanism of action. Thus, there are base analogs (for example 5-bromouracil, 2-aminopurine), mono- and bifunctional alkylating agents (for example monofunctional agents such as ethyl methyl sulfonate (EMS), dimethyl sulfate, or bifunctional agents such as dichloroethyl sulfite, mitomycin, nitrosoguanidine-dialkyl nitrosamine, N-nitrosoguanidine derivatives) or intercalating substances (for example acridine, ethidium bromide).

Examples of physical mutagens are ionizing radiations. Ionizing radiations are electromagnetic waves or corpuscular radiations which are capable of ionizing molecules, i.e. of removing electrons from them. The ions which remain are in most cases highly reactive so that they, in the event that they are formed in live tissue, are capable of inflicting great damage to the DNA and thereby inducing mutations (at low intensity). Examples of ionizing radiations are gamma radiation (photon energy of approximately one mega electron volt MeV), X-ray radiation (photon energy of several or many kilo electron volt keV) or else ultraviolet light (UV light, photon energy of over 3.1 eV). UV light causes the formation of dimers between bases, thymidine dimers are most common, and these give rise to mutations.

Examples of the generation of mutants by treating the seeds with mutagenizing agents may include ethyl methyl sulfonate (EMS) (Birchler, J. A. and Schwartz, D., Biochem. Genet. 17 (11-12), 1173 (1979); Hoffmann, G. R., Mutat. Res. 75 (1), 63 (1980)) or ionizing radiation there has now been added the use of biological mutagens, for example transposons (for example Tn5, Tn903, Tn916, Tn1000, May B. P. et al., Proc. Natl. Acad. Sci USA. 100 (20), 11541 (2003)) or molecular-biological methods such as the mutagenesis by T-DNA insertion (Feldman, K. A., Plant Journal 1, 71 (1991), Koncz, C., et al., Plant Mol. Biol. 20: 963-76 (1992)).

Domains can be identified by suitable computer programs such as, for example, SMART or InterPRO, for example as described in Andersen P., The Journal of Biol. Chemistry, 279, 38 or 39053, (2004) or Mudgil, Y., Plant Physiology, 134, 59, (2004), and literature cited therein. The suitable mutants can then be identified for example by TILLING (for example as described by Henikoff, S., et al., Plant Physiol. 135: 630-6 (2004)).

Additionally, it is also possible to increase the endogenous overexpression or activity of these sequences in a plant or organism by mutating a UTR region, such as the 5′-UTR, a promoter region, a genomic coding region for the active center, for binding sites, for localization signals, for domains, clusters and the like, such as, for example, of coding regions for the N-terminal, the FMO protein or the C-terminal domains. The endogenous expression or activity may be increased in accordance with the invention by mutations which affect the secondary, tertiary or quaternary structure of the protein.

The introduction and overexpression of a sequence according to the methods described herein into a plant or photosynthetic organism, or increasing or modifying or mutating an endogenous sequence, if appropriate of one or both untranslated regions, in a plant or photosynthetic organism is combined with increasing the polypeptide quantity, activity or function of other resistance factors, such as a Bax inhibitor 1 protein (BI-1), from Hordeum vulgare (GenBank Acc.-No.: AJ290421), from, Nicotiana tabacum (GenBank Acc.-No.: AF390556), rice (GenBank Acc.-No.: AB025926), Arabidopsis (GenBank Acc.-No.: AB025927) or tobacco and oilseed rape (GenBank Acc.-No.: AF390555, Bolduc N et al. (2003) Planta 216, 377 (2003)) or of ROR2 (for example from barley (GenBank Acc.-No.: AY246906), SnAP34 (for example, from barley (GenBank Acc.-No.: AY247208) and/or of the lumenal binding protein BiP for example from rice (GenBank Acc.-No. AF006825). An increase can be achieved for example, by mutagenesis or overexpression of a transgene, inter alia.

### Selectable Markers

In another embodiment, DNA constructs comprising a promoter operably linked to one or more FMO proteins may further comprise a selectable marker operably linked to a promoter. Selectable markers which confer a resistance to a metabolism inhibitor such as 2-deoxyglucose 6-phosphate (WO 98/45456), antibiotics or biocides, herbicides, for example kanamycin, G 418, bleomycin, hygromycin or phosphinotricin, may be included in the DNA construct. For example, DNA sequences which code for phosphinothricin acetyltransferases (PAT), which inactivate glutamine synthase inhibitors (bar and pat gene), 5-enolpyruvylshikimate-3-phosphate synthase (EPSP synthase genes) which confer resistance to Glyphosat® (N-phosphonomethyl glycine), the gox gene, which codes for the Glyphosat®-degrading enzyme (glyphosate oxidoreductase), the deh gene (coding for a dehalogenase which inactivates dalapon), and bxn genes which code for bromoxynil-degrading nitrilase enzymes, the aasa gene, which confers a resistance to the antibiotic spectinomycin, the streptomycin phosphotransferase (SPT) gene, which makes possible a resistance to streptomycin, the neomycin phosphotransferase (NPTII) gene, which confers a resistance to kanamycin or geneticidin, the hygromycin phosphotransferase (HPT) gene, which mediates a resistance to hygromycin, the acetolactate synthase gene (ALS), which mediates a resistance to sulfonylurea herbicides (for example mutated ALS variants with, for example, the S4 and/or Hra mutation), and the acetolactate synthase gene (ALS), which mediates a resistance to imidazolinone herbicides.

### Reporter Genes

Reporter genes may also be included in the DNA construct. Reporter genes are genes which code for easily quantifiable proteins and ensure via an intrinsic color or enzymic activity an assessment of the transformation efficiency or of the location or timing of expression (Schenborn E. and Groskreutz D. Mol Biotechnol.; 13(1):29 (1999) Reporter genes may include, but are not limited to, the green fluorescence protein (GFP) (Sheen et al. Plant Journal 8(5):777 (1995); Haselhoff et al Proc Natl Acad Sci USA 94(6):2122 (1997); Reichel et al. Proc Natl Acad Sci USA 93(12):5888 (1996); Tian et al. Plant Cell Rep 16:267 (1997); WO 97/41228; Chui et al. Curr Biol 6:325 (1996); Leffel et al. Biotechniques. 23(5):912-8 (1997)), the chloramphenicoltransferase, a luciferase (Ow et al. Science 234:856 (1986); Millar et al. Plant Mol Biol Rep 10:324 (1992)), the aequorin gene (Prasher et al. Biochem Biophys Res Commun 126(3):1259 (1985)), the [beta]-galactosidase, the R-locus gene, which codes for a protein which regulates the production of anthocyanin pigments (red coloration) in plant tissue and thus makes possible the direct analysis of the promoter activity without the addition of additional adjuvants or chromogenic substrates (Dellaporta et al., In: Chromosome Structure and Function: Impact of New Concepts, 18th Stadler Genetics Symposium, 11:263, (1988), with [beta]-glucuronidase (Jefferson et al., EMBO J., 6, 3901, 1987).

### Transformation

The introduction into a plant or organism of a DNA construct comprising, for example, the FMO protein (SEQ ID NO: 1-44) into a photosynthetic organism, plant, or plant part such as plant cells, plant tissue, and plant organs such as chloroplasts and seeds, can be carried out using vectors (for example the pROK2 vector, or the pCAMBIA vector) which comprise the DNA construct. The vectors may take the form of, for example, plasmids, cosmids, phages, and other viruses or Agrobacterium containing the appropriate vector may be used.

A variety of methods (Keown et al., Methods in Enzymology 185, 527(1990)) are available for the introduction of a desired construct into a plant or organism, which is referred to as transformation (or transduction or transfection). Thus, the DNA or RNA can be introduced for example, directly by means of microinjection or by bombardment with DNA-coated microparticles. Also, it is possible to chemically permeabilize the cell, for example using polyethylene glycol, so that the DNA can reach the cell by diffusion. The DNA can also be introduced into the cell by means of protoplast fusion with other DNA-comprising units such as minicells, cells, lysosomes or liposomes. A further suitable method of introducing DNA is electroporation, where the cells are reversibly permeabilized by means of an electrical pulse. Examples of such methods have been described in Bilang et al., Gene 100, 247 (1991); Scheid et al., Mol. Gen. Genet. 228, 104 (1991); Guerche et al., Plant Science 52, 111 (1987); Neuhause et al., Theor. Appl. Genet. 75, 30 (1987); Klein et al., Nature 327, 70(1987); Howell et al., Science 208, 1265 (1980); Horsch et al., Science 227, 1229 (1985); DeBlock et al., Plant Physiology 91, 694 (1989); “Methods for Plant Molecular Biology” (Weissbach and Weissbach, eds.) Academic Press Inc. (1988); and “Methods in Plant Molecular Biology” (Schuler and Zielinski, eds.) Academic Press Inc. (1989).

Binary vectors are capable of replicating in a variety of organisms including but not limited to E. coli and in agrobacterium. They may comprise a selectable marker gene and a linker or polylinker flanked by the right and left T-DNA border sequence. They can be transformed directly into agrobacterium (Holsters et al., Mol. Gen. Genet. 163, 181 (1978)). The selection marker gene, for example the nptII gene, which mediates resistance to kanamycin, permits transformed agrobacteria to be selected. The agrobacterium acts as the host organism and may already comprise a helper Ti plasmid with the vir region, for transferring the T-DNA to the plant cell. An agrobacterium thus transformed can be used for transforming plant cells. The use of T-DNA for the transformation of plant cells has been studied and described (EP 120 516; Hoekema, in “The Binary Plant Vector System”, Offsetdrukkerij Kanters B. V., Alblasserdam, Chapter V; An et al. EMBO J. 4, 277 (1985)). Various binary vectors are known and in some cases are commercially available, such as, for example, pBI101.2 or pBIN19 (Clontech Laboratories, Inc. USA).

In the event that DNA or RNA is injected or electroporated into plant cells, the plasmid used need not meet particular requirements. Simple plasmids such as those from the pUC series may be used. If intact plants are to be regenerated from the transformed cells, an additional selection marker gene may be located on the plasmid. Additional methods are described in Jones et al. (“Techniques for Gene Transfer”, in “Transgenic Plants”, Vol. 1, Engineering and Utilization, edited by Kung S. D. and Wu R., Academic Press, p. 128-143 (1993), and in Potrykus, Annu. Rev. Plant Physiol. Plant Molec. Biol. 42, 205 (1991)).

In plants, the herein described methods for the transformation and regeneration of plants from plant tissue or plant cells are exploited for the purposes of transient or stable transformation. Suitable methods are mainly protoplast transformation by means of polyethylene-glycol-induced DNA uptake, the biolistic method with the gene gun, known as the particle bombardment method, electroporation, the incubation of dry embryos in DNA-comprising solution, and microinjection. Transformation may also be effected by bacterial infection by means of Agrobacterium tumefaciens or Agrobacterium rhizogenes. The methods are further described for example in Horsch et al. Science 225, 1229 (1985). If agrobacteria are used for transformation, the DNA construct may be integrated into specific plasmids, which may either be a shuttle or intermediate vector or a binary vector. If a Ti or Ri plasmid is used for the transformation, at least the right border, but in most cases both the right and the left border, of the Ti or Ri plasmid T-DNA as flanking region is linked with the DNA construct to be introduced.

Stably transformed cells, i.e. those which comprise the DNA construct integrated into the DNA of the host cell, can be selected from untransformed cells when a selection marker is present (McCormick et al, Plant Cell Reports 5, 81 (1986)). For example, any gene which is capable of mediating a resistance to antibiotics or herbicides (such as kanamycin, G 418, bleomycin, hygromycin or phosphinothricin) may act as a marker. Transformed cells which express such a marker gene are capable of surviving in the presence of concentrations of a suitable antibiotic or herbicide which destroy an untransformed wild-type cells. Examples include the bar gene, which mediates resistance to the herbicide phosphinothricin (Rathore et al., Plant Mol. Biol. 21 (5), 871 (1993)), the nptII gene, which mediates resistance to kanamycin, the hpt gene, which mediates resistance to hygromycin, or the EPSP gene, which mediates resistance to the herbicide glyphosate.

Stably transformed cells can be also be selected for stable integration of the DNA construct using methods known in the art, such as restriction analysis and sequencing.

When a transformed plant cell has been generated, an intact plant can be obtained using methods known to one skilled in the art. An example of a starting material used are callus cultures. The formation of shoot and root from this as yet undifferentiated cell biomass can be induced in a known manner. The plantlets obtained can be planted out and bred. A person skilled in the art also knows methods for regenerating plant parts and intact plants from plant cells. For example, methods described by Fennell et al., Plant Cell Rep, 11, 567 (1992); Stoeger et al., Plant Cell Rep. 14, 273 (1995); Jahne et al., Theor. Appl. Genet. 89, 525 (1994), are used for this purpose.

The resulting plants can be bred and hybridized in the customary manner. Two or more generations should be cultivated in order to ensure that the genomic integration is stable and hereditary.

The term “overexpression”, as used herein, means that a given cell produces an increased number of a certain protein relative to a normal cell. The original wild-type expression level might be zero, i.e. absence of expression or immeasurable expression. It will be understood that the FMO protein that is overexpressed in the cells according to the methods of this disclosure can be of the same species as the plant cell wherein the overexpression is being carried out or it may be derived from a different species. In the case wherein the endogenous (sequence from the same species) FMO protein, is overexpressed as a transgene, the levels of the FMO protein are between 4 and 37 fold greater with respect to the same polypeptide which is endogenously produced by the plant cell. In the case wherein a heterologous (sequence from a different species) FMO protein, is overexpressed as a transgene, the levels of the heterologous FMO protein are between 4 and 37 fold greater than the levels of the endogenous FMO protein.

FMO proteins catalyze the oxidation of endogenous metabolites containing nucleophilic nitrogen, such as oxidation of trimethylamine (TMA) to trimethylamine N-oxide TMAO. The levels of TMAO can be determined by methods known in the art, including, for instance, the method described on PCT application WO20100348262 based on the reduction of TMAO to TMA in the presence of TiCl3 and detecting the amount of TMA formed in the reaction.

In another embodiment, transgenic plants overexpressing an FMO protein have between 1.1 and 3.4 fold increase in TMAO compared to wild-type.

In another embodiment, drought tolerant transgenic plants may be generated having a DNA construct stably integrated into said plants genome, wherein said DNA construct comprises an FMO protein coding sequence operably linked to a promoter, wherein said plant overexpresses said FMO protein between 4 and 37 fold greater than the level of FMO expression in non-transgenic plants, wherein said overexpression of said FMO protein catalyzes the oxidation of endogenous metabolites containing nucleophilic nitrogen, and wherein said transgenic plant has between 1.1 and 3.4 fold greater trimethylamine N-oxide.

In another embodiment of the disclosure, the overexpression, either constitutive or induced, of an FMO protein in a plant or photosynthetic organism mediates increased TMAO and produces a drought tolerant plant or photosynthetic organism.

### Drought Stress

Drought stress in plants may be recognized or identified by comparing a change in plant phenotypes between plants which have been exposed to drought stress conditions and plants which have not been exposed to the same drought stress conditions. Drought stress in a plant or photosynthetic organism may be indicated by a change in one or more of the following plant phenotypes, which can serve as indicators of the drought stress in plants: (1) germination percentage, (2) seedling establishment rate, (3) number of healthy leaves, (4) plant length, (5) plant weight, (6) leaf area, (7) leaf color, (8) number or weight of seeds or fruits, (9) quality of harvests, (10) flower setting rate or fruit setting rate, (11) chlorophyll fluorescence yield, (12) water content, (13) leaf surface temperature, and (14) transpiration capacity. Other indicators not listed may also be included.

Drought stress may be quantified as the “intensity of stress” where intensity of stress is represented as following: “Intensity of stress”=100×“any one of plant phenotypes in plants which have not been exposed to drought stress”/“the plant phenotype in plants which have been exposed to drought stress”. The methods described herein are applied to plants that have been exposed to or to be exposed to drought stress conditions whose intensity of stress represented by the above equation is from 105 to 450. The description of a range should be considered to have specifically disclosed all the possible subranges as well as individual numerical values within that range. In a plant exposed to drought stress conditions, an influence may be recognized on at least one of the above phenotypes. That is, observed as: (1) decrease in germination percentage, (2) decrease in seedling establishment rate, (3) decrease in number of healthy leaves, (4) decrease in plant length, (5) decrease in plant weight, (6) decrease in leaf area increasing rate, (7) leaf color fading, (8) decrease in number or weight of seeds or fruits, (9) deterioration in quality of harvests, (10) decrease in flower setting rate or fruit setting rate, (11) decrease in chlorophyll fluorescence yield, (12) decrease in water content, (13) increase in leaf surface temperature, or (14) decrease in transpiration capacity, among others, and the magnitude of the drought stress in the plant can be measured using that as an indicator.

Another embodiment of the disclosure also relates to a transgenic tissue culture of cells produced from transgenic plants overexpressing an FMO protein, wherein the cells of the tissue culture are produced from a plant part chosen from leaves, pollen, embryos, cotyledons, hypocotyl, meristematic cells, roots, root tips, pistils, anthers, flowers, and stems, and wherein said tissue culture of cells overexpresses an FMO protein between 4 and 37 fold greater compared tissue cultures of cells derived from wild-type plants.

An additional embodiment of the disclosure relates to transgenic plants regenerated from tissue cultures of cells overexpressing an FMO protein between 4 and 37 fold greater compared to wild-type plants.

In an additional embodiment of the disclosure, transgenic plants overexpressing an FMO protein compared to wild-type plants have an increased biomass under non-stressed conditions compared to wild-type plants.

In an additional embodiment of the disclosure, transgenic plants overexpressing an FMO protein compared to wild-type plants have an increased seed yield as a total of the seed weight under non-stressed conditions compared to wild-type plants.

An additional embodiment of the disclosure include methods for producing plants or photosynthetic organisms tolerant to drought stress. These methods include the application of an effective amount of an organic compound such as trimethylamine N-oxide di-hydrate to plants or photosynthetic organisms to produce a plant or photosynthetic organism tolerant to drought stress.

One or more embodiments described herein may further provide methods for producing a drought tolerant plant or photosynthetic organism which comprises applying an effective enough amount of TMAO di-hydrate to a plant or organism that has been exposed to or to be exposed to drought stress conditions. This method may further include a seed treatment application, a spray treatment or an irrigation treatment of TMAO di-hydrate. As an example an effective amount of TMAO di-hydrate seed treatment may include a seed treatment of TMAO di-hydrate in an amount from 0.1 to 1000 g per 1 kg seed or 0.1 to 100 g per liter of spray treatment or irrigation treatment. When incorporated into the entire soil, an effective amount of TMAO di-hydrate may range from 0.1 to 1.000 g or 1 to 500 g, per 1.000 m² of soil. In the treatment of seedlings, an example of the weight of the TMAO di-hydrate per seedling may range from 0.01 to 20 mg, including 0.5 to 8 mg. In the treatment of the soil before or after sowing seedlings, the weight of the TMAO di-hydrate per 1.000 m² may range from 0.1 to 1000 g, including from 10 to 100 g.

TMAO di-hydrate may be applied to a variety of plants in various forms or sites, such as foliage, buds, flowers, fruits, ears or spikes, seeds, bulbs, stem tubers, roots and seedlings. As used herein, bulbs mean discoid stem, rhizomes, root tubers, and rhizophores. In the present disclosure, TMAO di-hydrate may also be applied to cuttings and sugar cane stem cuttings.

The following are examples of the growing sites of plants include soil before or after sowing plants. When TMAO di-hydrate is applied to plants or growing sites of plants, the TMAO di-hydrate is applied to the target plants once or more. TMAO di-hydrate may be applied as a treatment to foliage, floral organs or ears or spikes of plants, such as foliage spraying; treatment of seeds, such as seed sterilization, seed immersion or seed coating; treatment of seedlings; treatment of bulbs; and treatment of cultivation lands of plants, such as soil treatment. TMAO di-hydrate may be applied only to specific sites of plants, such as floral organ in the blooming season including before blooming, during blooming and after blooming, and the ear or spike in the earing season, or may be applied to entire plants.

TMAO di-hydrate may be applied as a soil treatment in the form a spray onto soil, soil incorporation, and perfusion of a chemical liquid into the soil (irrigation of chemical liquid, soil injection, and dripping of chemical liquid). The placement of TMAO di-hydrate during soil treatment includes but is not limited to planting hole, furrow, around a planting hole, around a furrow, entire surface of cultivation lands, the parts between the soil and the plant, area between roots, area beneath the trunk, main furrow, growing box, seedling raising tray and seedbed, seedling raising. TMAO di-hydrate soil treatment may be before seeding, at the time of seeding, immediately after seeding, raising period, before settled planting, at the time of settled planting, and growing period after settled planting.

Alternatively, an irrigation liquid may be mixed with the TMAO di-hydrate in advance and, for example, used for treatment by an appropriate irrigating method including the irrigation method mentioned above and the other methods such as sprinkling and flooding. TMAO di-hydrate may also be applied by winding a crop with a resin formulation processed into a sheet or a string, putting a string of the resin formulation around a crop so that the crop is surrounded by the string, and/or laying a sheet of the resin formulation on the soil surface near the root of a crop.

In another embodiment, TMAO di-hydrate may be used for treating seeds or bulbs as well as a TMAO di-hydrate spraying treatment for seeds in which a suspension of TMAO di-hydrate is atomized and sprayed on a seed surface or bulb surface. A smearing treatment may also be used in where a wettable powder, an emulsion or a flowable agent of the TMAO di-hydrate is applied to seeds or bulbs with a small amount of water added or applied as is without dilution. In addition, an immersing treatment may be used in which seeds are immersed in a solution of the TMAO di-hydrate for a certain period of time, film coating treatment, and pellet coating treatment.

TMAO di-hydrate may be used for the treatment of seedlings, including spraying treatment comprised of spraying the entire seedlings with a dilution having a proper concentration of active ingredients prepared by diluting the TMAO di-hydrate with water. As with seed treatment, an immersing treatment may also be used comprised of immersing seedlings in the dilution, and coating treatment of adhering the TMAO di-hydrate formulated into a dust formulation to the entire seedlings.

TMAO di-hydrate may be treated to soil before or after sowing seedlings including spraying a dilution having a proper concentration of active ingredients prepared by diluting TMAO di-hydrate with water and applying the mixture to seedlings or the soil around seedlings after sowing seedlings. A spray treatment of TMAO di-hydrate formulated into a solid formulation such as a granule to soil around seedlings at sowing seedlings may also be used.

In another embodiment, TMAO di-hydrate may be applied for efficient water usage, where normal yields are produced with less water input. The term “efficient water use” may be applied to a plant that is induced to produce normal yields under conditions where less water than is customary or average for an area or a plant is applied to a plant.

In another embodiment, TMAO di-hydrate may be applied allowing for the production of plants and photosynthetic organisms wherein the endogenous level of TMAO is between 1.1 and 9.9 fold greater when compared to photosynthetic organisms and plants that have not been treated with TMAO di-hydrate.

### Detection of Endogenous TMAO

There are a number of methods known in the art to detect and quantify the level of endogenous TMAO content in plants. For example, one may quantify TMAO by NMR spectrometry, such as, for example, using a Bruker Advance DRX 500 MHz spectrometer equipped with a 5 mm inverse triple resonance probe head. A known concentration of [3-(trimethylsilyl) propionic-2,2,3,3-d4 acid sod. salt, (TSP-d4)] can be used as an internal reference. Additional TMAO detection methods include, but are not limited to Trichloro acetic acid, 5% wt/v extraction using ferrous sulphate and EDTA (Wekell, J. C., Barnett, H., 1991. New method for analysis of trimethyl-amine oxide using ferrous sulphate and EDTA. J. Food Sci. 56, 132-138 . . . ) or using capillary gas chromatography-mass spectrometry (daCosta K A, Vrbanac J J, Zeisel S H. The measurement of dimethylamine, trimethylamine, and trimethylamine N-oxide using capillary gas chromatography-mass spectrometry (Anal. Biochem. 990; 187:234-239).

In another embodiment, TMAO di-hydrate may be applied allowing for the production of plants and photosynthetic organisms with more biomass when compared to plants and photosynthetic organisms that have not been treated with TMAO di-hydrate.

In another embodiment, TMAO di-hydrate may be applied allowing for the production of plants and photosynthetic organisms with greater survival rate compared to plants and photosynthetic organisms that have not been treated with TMAO di-hydrate.

In another embodiment, TMAO di-hydrate may be applied allowing for the production of plants with greater seed production compared to plants have not been treated with TMAO di-hydrate.

In another embodiment, TMAO di-hydrate may be applied allowing for the production of plants with greater fruit production compared to plants that have not been treated with TMAO di-hydrate.

In another embodiment, TMAO di-hydrate may be applied allowing for the production of plants with greater inflorescence weight compared to plants have not been treated with TMAO di-hydrate.

In another embodiment, TMAO di-hydrate may be applied allowing for the production of plants and photosynthetic organisms with greater yield compared to plants and photosynthetic organisms that have not been treated with TMAO di-hydrate.

In another embodiment, TMAO di-hydrate may be applied allowing for the production of plants having greater average dry weight compared to plants that have not been treated with TMAO di-hydrate.

In another embodiment, TMAO di-hydrate may be applied allowing for the production of plants and photosynthetic organisms with more chlorophyll compared to plants and photosynthetic organisms that have not been treated with TMAO di-hydrate.

### EXAMPLES

The following examples are provided to illustrate further the various applications and are not intended to limit the invention beyond the limitations set forth in the appended claims.

The recombinant nucleic acid molecules described herein comprise the following elements: regulatory sequences of a promoter which is active in plant cells, a DNA sequence in operative linkage therewith, if appropriate, regulatory sequences which, in the plant cell, may act as transcription, termination and/or polyadenylation signals in operable linkage therewith, and further comprising an FMO protein coding sequence in operable linkage with at least one genetic control element (for example a promoter) which enables overexpression in plants.

### Example 1

### DNA Constructs for the Overexpression of an FMO Protein

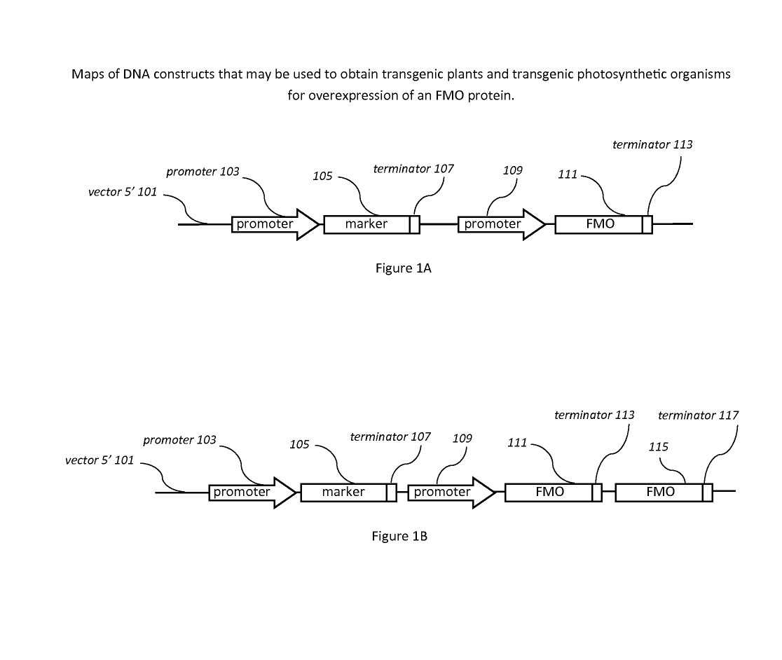

Shown in FIG. 1A is an example map of a DNA construct that may be used to obtain transgenic plants and transgenic photosynthetic organisms for overexpression of an FMO protein. A vector 101 holds the DNA construct comprising a promoter 103 operably linked to a marker 105 having a terminator sequence 107. Downstream is another promoter 109 operably linked to an FMO protein coding sequence 111 having a terminator sequence 113. As shown here, two different terminator sequences are used, but as will be understood by one skilled in the art, the same terminator sequences may also be used.

Shown in FIG. 1B is an example map of a DNA construct that may be used to obtain transgenic plants and transgenic photosynthetic organisms for overexpression of two or more FMO proteins. A vector 101 holds the DNA construct comprising a promoter 103 operably linked to a marker 105 having a terminator sequence 107. Downstream is another promoter 109 operably linked to two FMO protein coding sequences 111, 115 each having a terminator sequence 113, 117. As shown in FIG. 1B, two different FMO protein coding sequences are used, but as will be understood by one skilled in the art the FMO protein coding sequences may be the same or different.

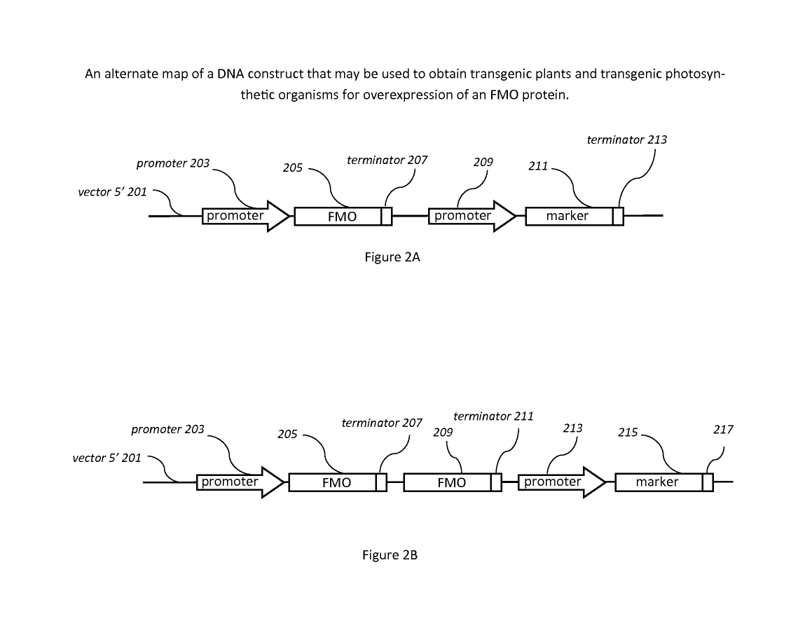

Shown in FIG. 2A is an example of an alternate map of a DNA construct that may be used to obtain transgenic plants and transgenic photosynthetic organisms for overexpression of an FMO protein. Here, the marker sequence is downstream of the FMO protein coding sequence. A vector 201 holds the DNA construct comprising a promoter 203 operably linked to an FMO protein coding sequence 205 having a terminator sequence 207. This is followed by a subsequent promoter 209 operably linked to a marker 211 having a terminator sequence 213. As shown here, two different terminator sequences are used, but as will be understood by one skilled in the art, the same terminator sequences may also be used.

Shown in FIG. 2B is an example of an alternate map of a DNA construct that may be used to obtain transgenic plants and transgenic photosynthetic organisms for overexpression of two or more FMO proteins. A vector 201 holds the DNA construct comprising a promoter 203 operably linked to two FMO protein coding sequences 205, 209 each having a terminator sequence 207, 211. This is followed by a subsequent promoter 213 operably linked to a marker 215 having a terminator sequence 217.

A variety of seeds or bulbs may be used in the methods described herein including but are not limited to plants in the families' Solanaceae and Cucurbitaceae, as well as plants selected from the plant genera Calibrachoa, Capsicum, Nicotiana, Nierembergia, Petunia, Solanum, Cucurbita, Cucumis, Citrullus, Glycine, such as Glycine max (Soy), Calibrachoa x hybrida, Capsicum annuum (pepper), Nicotiana tabacum (tobacco), Nierenbergia scoparia (cupflower), Petunia, Solanumlycopersicum (tomato), Solanum tuberosum (potato), Solanum melongena (eggplant), Cucurbita maxima (squash), Cucurbita pepo (pumpkin, zucchini), Cucumis metuliferus (Horned melon) Cucumis melo (Musk melon), Cucumis sativus (cucumber) and Citrullus lanatus (watermelon). Various monocotyledonous plants, in particular those which belong to the family Poaceae, may be used with the methods described herein, including but not limited to, plants selected from the plant genera Hordeum, Avena, Secale, Triticum, Sorghum, Zea, Saccharum, Oryza, Hordeum vulgare (barley), Triticum aestivum (wheat), Triticum aestivum subsp. spelta (spelt), Triticale, Avena sativa (oats), Secale cereale (rye), Sorghum bicolor (sorghum), Zea mays (maize), Saccharum officinarum (sugarcane) and Oryza sativa (rice).

Additional examples of plants in which drought stress tolerance may be produced using the methods described herein include the following crops: rice, corn, canola, soybean, wheat, buckwheat, beet, rapeseed, sunflower, sugar cane, tobacco, and pea, etc.; vegetables: solanaceous vegetables such as paprika and potato; cucurbitaceous vegetables; cruciferous vegetables such as Japanese radish, white turnip, horseradish, kohlrabi, Chinese cabbage, cabbage, leaf mustard, broccoli, and cauliflower, asteraceous vegetables such as burdock, crown daisy, artichoke, and lettuce; liliaceous vegetables such as green onion, onion, garlic, and asparagus; ammiaceous vegetables such as carrot, parsley, celery, and parsnip; chenopodiaceous vegetables such as spinach, Swiss chard; lamiaceous vegetables such as Perilla frutescens, mint, basil; strawberry, sweet potato, Dioscorea japonica, colocasia; flowers; foliage plants; grasses; fruits: pomaceous fruits (apple, pear, Japanese pear, Chinese quince, quince, etc.), stone fleshy fruits (peach, plum, nectarine, Prunus mume, cherry fruit, apricot, prune, etc.), citrus fruits (Citrus unshiu, orange, tangerine, lemon, lime, grapefruit, etc.), nuts (chestnuts, walnuts, hazelnuts, almond, pistachio, cashew nuts, macadamia nuts, etc.), berries (blueberry, cranberry, blackberry, raspberry, etc.), grape, kaki fruit, olive, Japanese plum, banana, coffee, date palm, coconuts, etc.; and trees other than fruit trees; tea, mulberry, flowering plant, roadside trees (ash, birch, dogwood, Eucalyptus, Ginkgo biloba, lilac, maple, Quercus, poplar, Judas tree, Liquidambar formosana, plane tree, zelkova, Japanese arborvitae, fir wood, hemlock, juniper, Pinus, Picea, and Taxus cuspidata).

### Example 2

### DNA Construct for the Constitutive Overexpression of the RCI5 FMO Protein in Arabidopsis thaliana Plants

For FMO protein overexpression, transgenic Arabidopsis plants overexpressing the FMO GS-OX5 gene (SEQ ID NO: 1 or SEQ ID NO: 2) and described as RCI5-OE (ES 2347399B1) (FMO3X and FMO8X) were obtained using the methods described below.

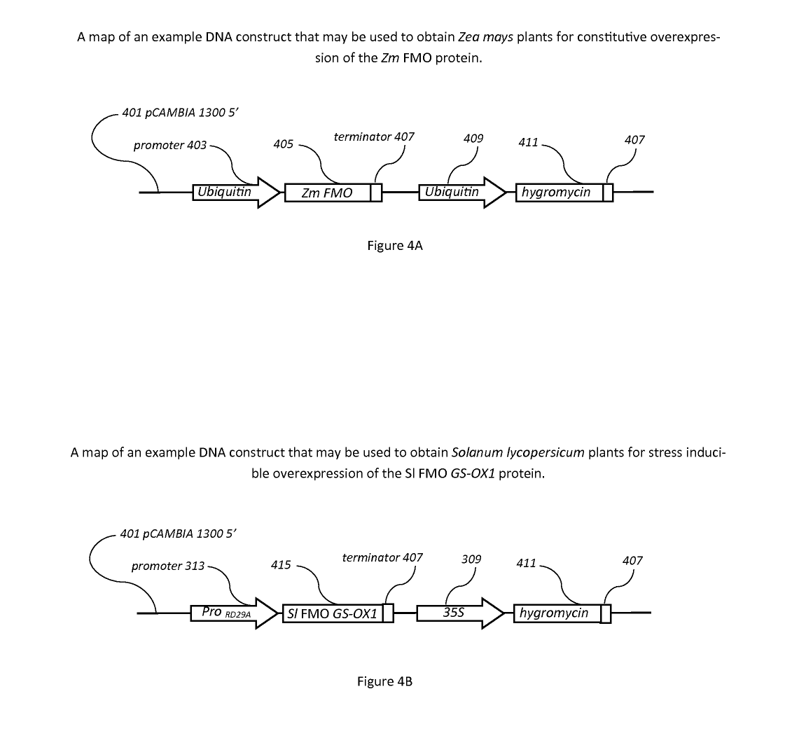

RCI5 cDNA was ligated downstream of the CaMv35S promoter in the pROK2 vector (Baulcombe et al., 1986) (shown in the construct of FIG. 4A), to obtain transgenic plants. Once the presence of the construct (such as the construct described in FIG. 4A and FIG. 4B) was verified in the recombinant plasmid by DNA sequencing, DNA constructs were introduced into the Agrobacterium tumefaciens strain C58C1 (Deblaere et al., 1985).

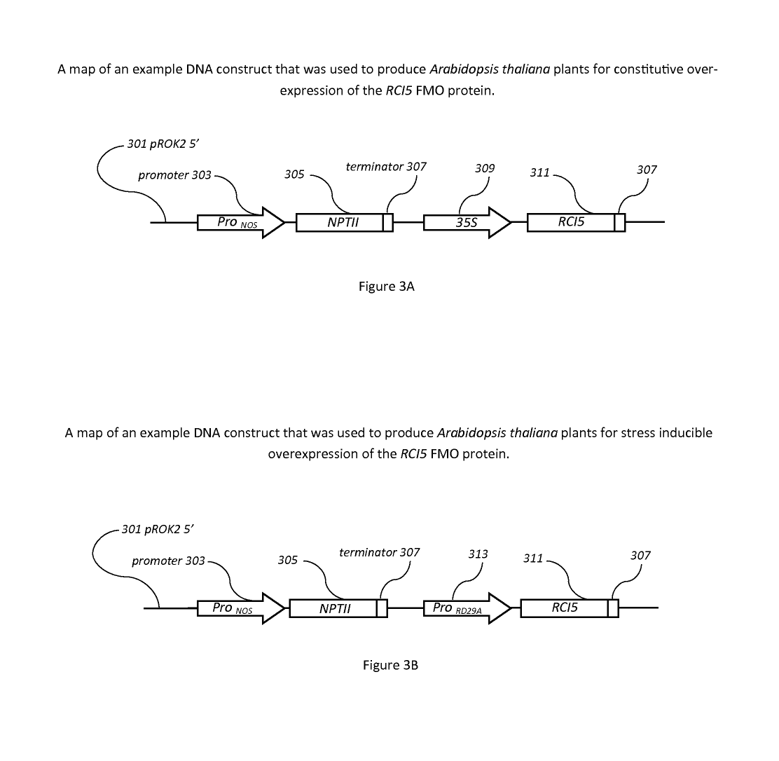

Shown in FIG. 3A is a map of a DNA construct that was used to produce Arabidopsis thaliana plants for constitutive overexpression of the RCI5 FMO protein. Staring at the 5′ end, a vector 301, pROK2 holds a DNA construct comprising a constitutive promoter coding sequence 303, PRO_{NOS}, operably linked to a selectable marker 305, NPTII having a terminator sequence 307 on the 3′end of the selectable marker 305. FMO protein RCI5 311 cDNA (SEQ ID NO: 1 or SEQ ID NO:2) was ligated downstream of and operably linked to the constitutive CaMv35S (35S) promoter 309. A transcription termination sequence 307 is present on the 3′end of the FMO RCI5 311.

Once the presence of the construct was verified in the recombinant plasmid by DNA sequencing, plasmids were introduced into the Agrobacterium tumefaciens strain C58C1 (Deblaere et al., 1985). Transformation of Arabidopsis Col was performed following the floral dip method (Clough and Bent, 1998).

The plants were sown in plastic pots containing the same amount of water saturated substrate. Trays containing 16 pots with 5 plants per pot were placed in a grow chamber under short-day light conditions until the plants developed 12 leaves. Then, the trays were transferred to the greenhouse under long-day light conditions and the pots were individually placed in transparent plastic glasses in order to avoid water spillage during irrigations. Normal irrigated plants for each genotype were also placed on the trays, as controls. A total of 4 trays were used, with differently distributed genotypes within each tray. Under normal growth conditions, no phenotypic differences were observed among genotypes.

RNA from three week old T2 plants grown at 20° C. was extracted and 20 μg of total RNA was loaded per lane for a northern hybridization with an RCI5 probe to screen for the highest levels of FMO expression in the T2 generation plants. As loading control a ribosomal RNA 18S gene probe was used. As used herein, T2 refers to the F₂ generation of transgenic plants.

### Example 3

### DNA Construct for Stress Inducible Overexpression of the RCI5 FMO Protein in Arabidopsis thaliana Plants

Shown in FIG. 3B is a map of a DNA construct that was used to produce Arabidopsis thaliana plants for stress inducible overexpression of the RCI5 FMO protein. Staring at the 5′ end, a vector 301, pROK2 holds a DNA construct comprising a constitutive promoter coding sequence 303, PRO_{NOS}, operably linked to a selectable marker 305, NPTII having a terminator sequence 307 on the 3′end of the selectable marker 305. A stress inducible promoter 313, Pro_{RD29A} is operably linked to FMO protein coding sequence 311 RCI5 (SEQ ID NO: 1 or SEQ ID NO: 2) having a transcription termination sequence 307 on the 3′end of the FMO protein coding sequence.

Once the presence of the construct was verified in the recombinant plasmid by DNA sequencing, plasmids were introduced into the Agrobacterium tumefaciens strain C58C1 (Deblaere et al., 1985). Transformation of Arabidopsis Col was performed following the floral dip method (Clough and Bent, 1998).

The plants were sown in plastic pots containing the same amount of water saturated substrate. Trays containing 16 pots with 5 plants per pot were placed in a grow chamber under short-day light conditions until the plants developed 12 leaves. Then, the trays were transferred to the greenhouse under long-day light conditions and the pots were individually placed in transparent plastic glasses in order to avoid water spillage during irrigations. Normal irrigated plants for each genotype were also placed on the trays, as controls. A total of 4 trays were used, with differently distributed genotypes within each tray. Under normal growth conditions, no phenotypic differences were observed among genotypes.

RNA from three week old T2 plants grown at 20° C. was extracted and 20 μg of total RNA was loaded per lane for a Northern hybridization with an RCI5 probe to screen for the highest levels of FMO expression in the T2 generation plants. As loading control a ribosomal RNA 18S gene probe was used.

### Example 4

### DNA Construct for Constitutive Overexpression of the Zm FMO Protein in Zea mays Plants

Shown in FIG. 4A is a map of a DNA construct that may be used to obtain Zea mays plants for constitutive overexpression of the Zm FMO protein. Staring at the 5′ end, a vector 401, pCAMBIA 1300 holds a DNA construct comprising a constitutive promoter coding sequence 403, Ubiquitin, operably linked to FMO protein coding sequence 405 Zm FMO (SEQ ID NO: 25 or SEQ ID NO: 26) having a transcription termination sequence 407 on the 3′end of the FMO protein coding sequence. This is followed by a constitutive promoter 409, Ubiquitin operably linked to a selectable marker 411, hygromycin having a terminator sequence 407 on the 3′end of the selectable marker 411.

Once the presence of the construct is verified in the recombinant plasmid by DNA sequencing, plasmids can be introduced into the Agrobacterium tumefaciens strain C58C1 (Deblaere et al., 1985). Transformation of Zea mays can be performed following the floral dip method (Clough and Bent, 1998).

The plants can be sown in plastic pots containing the same amount of water saturated substrate and placed in a grow chamber under short-day light conditions until the plants developed 12 leaves. Then, the trays can be transferred to the greenhouse under long-day light conditions and the pots can be individually placed in transparent plastic glasses in order to avoid water spillage during irrigations. Normal irrigated plants for each genotype can also be placed on the trays, as controls.

RNA from three week old T2 plants grown at 20° C. can be extracted and 20 μg of total RNA can be loaded per lane for a Northern hybridization with an RCI5 probe to screen for the highest levels of FMO expression in the T2 generation plants. As loading control a ribosomal RNA 18S gene probe can be used.

### Example 5

### DNA Construct for Stress Inducible Overexpression of the Sl FMO GS-OX1 Protein in Solanum lycopersicum Plants

Shown in FIG. 4B is a map of an example DNA construct that may be used to obtain Solanum lycopersicum plants for stress inducible overexpression of the Sl FMO GS-OX1 protein. Staring at the 5′ end, a vector 401, pCAMBIA 1300 holds a DNA construct comprising a stress inducible promoter coding sequence 313, Pro_{RD29A}, operably linked to FMO protein coding sequence 415 Sl FMO GS-OX1 (SEQ ID NO: 35, SEQ ID NO: 36, SEQ ID NO: 37 or SEQ ID NO: 38) having a transcription termination sequence 407 on the 3′end of the FMO protein coding sequence. This is followed by a constitutive promoter 309, 35S operably linked to a selectable marker 411, hygromycin having a terminator sequence 407 on the 3′end of the selectable marker 411.

Once the presence of the construct is verified in the recombinant plasmid by DNA sequencing, plasmids can be introduced into the Agrobacterium tumefaciens strain C58C1 (Deblaere et al., 1985). Transformation of Solanum lycopersicum can be performed following the floral dip method (Clough and Bent, 1998).

The plants can be sown in plastic pots containing the same amount of water saturated substrate and placed in a grow chamber under short-day light conditions until the plants developed 12 leaves. Then, the trays can be transferred to the greenhouse under long-day light conditions and the pots can be individually placed in transparent plastic glasses in order to avoid water spillage during irrigations. Normal irrigated plants for each genotype can also be placed on the trays, as controls.

RNA from three week old T2 plants grown at 20° C. can be extracted and 20 μg of total RNA can be loaded per lane for a northern hybridization with an RCI5 probe to screen for the highest levels of FMO expression in the T2 generation plants. As loading control a ribosomal RNA 18S gene probe can be used.

### Example 6

### Overexpression of an FMO Protein in Arabidopsis thaliana Plants

T2 plants were grown at 20° C. under long day conditions. RNA was extracted from three week old plants. 50 plants from each group, wild-type, FMO8X, and FMO3X, (150 plants total) were pooled and RNA was extracted from each pool of 50. 20 μg of total RNA was loaded per lane for a northern hybridization with an RCI5 probe to screen for the highest levels of FMO expression in the T2 generation plants. As loading control a ribosomal RNA 18S gene probe was used. Lines that exhibited high levels of RCI5 were further analyzed by real-time PCR.

cDNA Library Preparation and Real-Time PCR

Total RNA was extracted from Wild-type (Col) and RCI5-OE (lines FMO8X and FMO3X) 12-day-old plants, grown in MS medium supplemented with 1% sucrose, using the Purezol reagent (Bio-Rad) according to the manufacturer's protocol. RNA samples were treated with DNase I (Roche) and quantified with a Nanodrop spectrophotometer (Thermo 4943 Scientific). For real-time qPCRs, cDNAs were prepared with the iScript cDNA synthesis kit (Bio-Rad) and then amplified using the Bio-Rad iQ2 thermal cycler, the SsoFast EvaGreen Supermix (Bio-Rad), and gene-specific primers. The relative expression values were determined using the AT4G24610 gene as a reference. All reactions were realized in triplicate employing three independent RNA samples. Values were statistically analyzed using the GraphPad Prism6 (GraphPad Software) statistical analysis software.

Table 1 below shows the relative amount of FMO RCI5 GS-OX5 RNA determined by real-time PCR analysis in wild-type and two transgenic lines, FMO8X and FMO3X. Column one shows the genotype, column two shows the relative level of RCI5 RNA, column three shows the mean of the three repeated experiments, column four shows the standard error, and column 5 shows the standard deviation (S.D.).

TABLE 1
RC15 RNA levels in wild-type and transgenic
lines quantified by real-time PCR analysis

| Geno- | Relative | | | |
|---|---|---|---|---|
| type | RC15 RNA | Mean | S.E. | S.D. |
| WT | 1 | 1 | 0 | 0 |
| | 1 | | | |
| | 1 | | | |
| FMO8X | 29.34 | 32.22 | 1.5 | 2.5 |
| | 34.12 | | | |
| | 33.22 | | | |
| FMO3X | 19 | 15.24 | 3.0 | 5.2 |
| | 17.44 | | | |
| | 9.29 | | | |

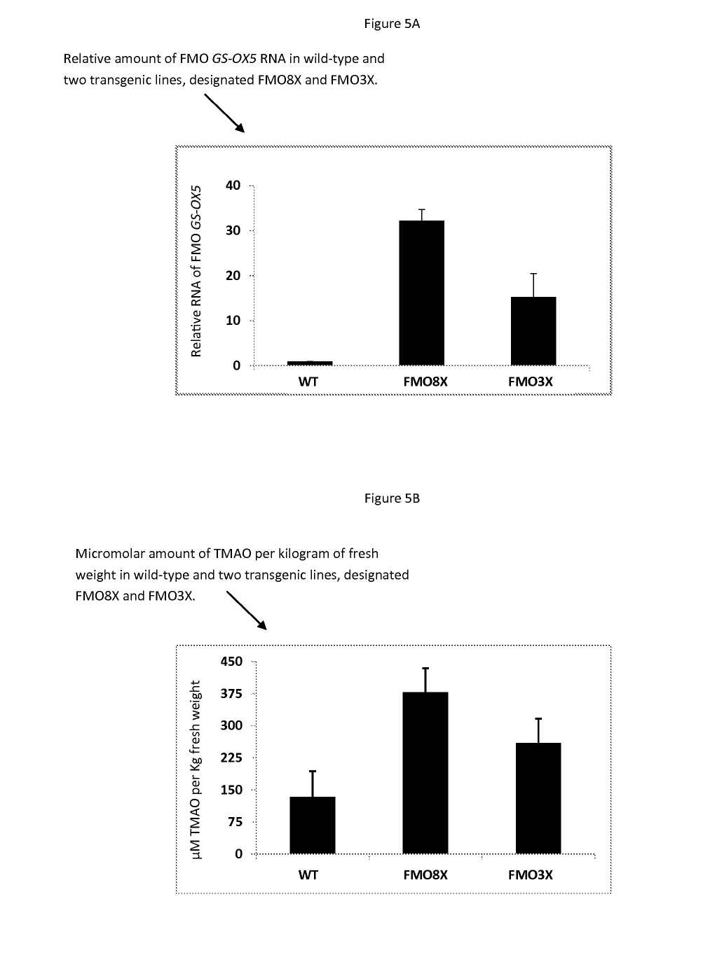

FIG. 5A shows a bar graph of the mean values represented in Table 1. As shown by Table 1 and FIG. 5A, transgenic lines FMO8X and FMO3X have an average fold increase in RC15 expression of 32.22 and 15.24, respectively. Taking into account the standard deviation, transgenic Arabidopsis plants of the present disclosure exhibit a range of between 4 and 37 fold increase in RC15 expression compared to wild-type.

### Example 7

### Overexpression of FMO Proteins Correlates with an Increase in TMAO

TMAO content in plants was determined by harvesting three leaves per treatment and freezing them in liquid nitrogen before the NMR determination. At least three independent plants were analyzed per experiment. TMAO content in plant extracts was quantified by NMR spectrometry using a Bruker Advance DRX 500 MHz spectrometer equipped with a 5 mm inverse triple resonance probe head. A known concentration of [3-(trimethylsilyl) propionic-2,2,3,3-d4 acid sod. salt, (TSP-d4)] was used as internal reference. All experiments were conducted at 298K and the data were acquired and processed using the same parameters. Spectra processing were performed on PC station using Topspin 2.0 software (Bruker).

Table 2 below shows that overexpression of FMO RC15 GS-OX5 in transgenic Arabidopsis increases constitutive levels of TMAO, and that this increase is dependent upon the level of FMO overexpression, as line FMO8X, which has a higher level of RC15 RNA (Table 1), exhibits a greater level of TMAO compared to line FMO3X and wild-type. Furthermore, line FMO3X, which has a higher level of RC15 RNA (Table 1) than wild-type, also exhibits a greater level of TMAO compared to wild-type. Three week old Arabidopsis plants were used for TMAO measurements. Data are expressed as the means of three independent experiments where 50 plants were pooled from each group: wild-type, FMO8X or FMO3X. Plants were grown at 20° C. under long day, non-stressed conditions. Column one shows the genotype, column two shows the concentration of TMAO expressed as micromole (μM) of TMAO per kilogram (kg) of fresh weight (FW), column three shows the average concentration of endogenous TMAO, column four shows the standard error (S.E.), column 5 shows the standard deviation (S.D.), and column 6 shows the mean fold change.

TABLE 2
TMAO levels in wild-type and transgenic lines quantified by NMR

| | [TMAO] | Mean [TMAO] | | | Mean fold |
|---|---|---|---|---|---|
| Genotype | uM | uM | S.E. | S.D. | change |
| WT | 128.10 | 134.03 | 3.5 | 6.00 | 1 |
| | 133.90 | | | | |
| | 140.10 | | | | |
| FMO8X | 313.68 | 377.80 | 32.5 | 56.23 | 2.82 |
| | 418.72 | | | | |
| | 401.00 | | | | |
| FMO3X | 206.58 | 260.08 | 32.6 | 56.55 | 1.94 |
| | 319.25 | | | | |
| | 254.40 | | | | |

FIG. 5B is a bar graph of the data represented in Table 2. As shown by Table 2 and FIG. 5B, wild-type plants have on average 134 μM TMAO per kg of fresh weight, whereas transgenic line FMO8X has an average 377.8 μM TMAO per kg of fresh weight, which is an average 2.82 fold increase, with a range of between 2.24 and 3.23 fold increase. Transgenic line FMO3X has an average 260.08 μM TMAO per kg of fresh weight, which is a 1.94 fold increase, with a range of between 1.47 and 2.49 fold increase. With the standard deviation, transgenic Arabidopsis lines of the present disclosure exhibit a range of between 150 μM TMAO per kg of fresh weight and 475 μM TMAO per kg of fresh weight, and have a range of between 1.1 and 3.4 fold increase in TMAO.

### Example 8

### Transgenic Arabidopsis Plants Overexpressing an FMO Protein are Drought Tolerant

To examine the drought stress tolerance of transgenic lines FMO3X and FMO8X, Arabidopsis plants were grown for 3 weeks under short day (10 hours light, 14 hours dark, 21° C. light and 20° C. at night, 65% humidity) conditions. After the 3 weeks the plants were not watered until the pots completely lost their moisture and the plants were extremely wilted. Then, they were watered, and the plants were left to lose their moisture completely again for three consecutive cycles of watering after wilting.

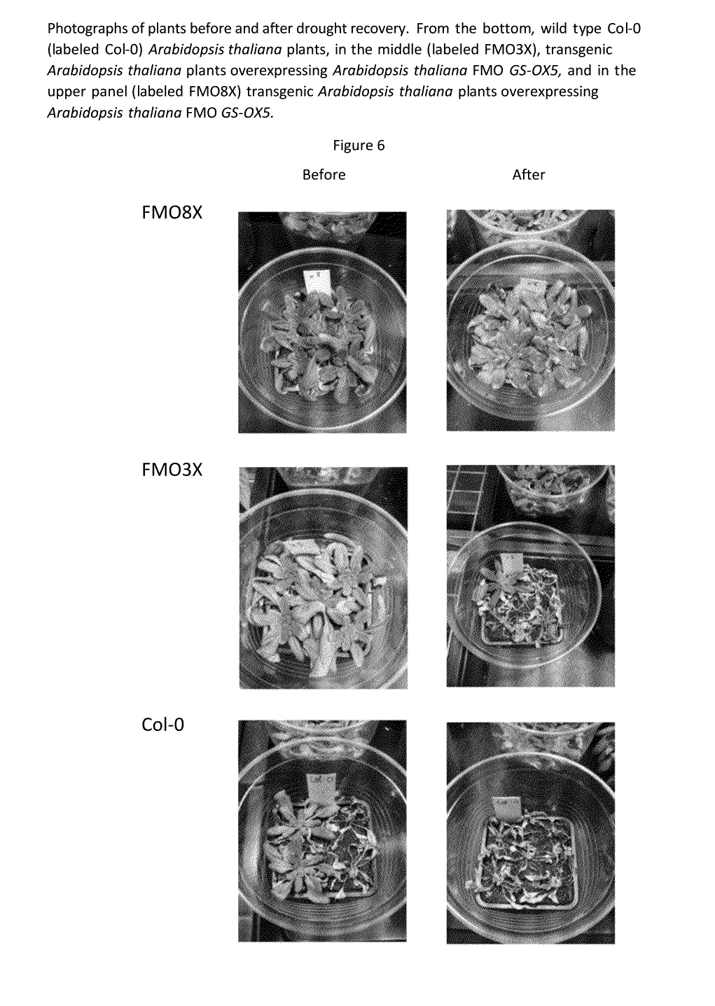

Shown in FIG. 6 are photographs of plants before and after the third drought recovery. From the bottom, wild-type Col-0 Arabidopsis thaliana plants (labeled Col-0), transgenic Arabidopsis thaliana T2 plants derived from line FMO3X (labeled FMO3X, middle), and transgenic Arabidopsis thaliana T2 plants derived from line FMO8X (labeled FMO8X, top) are shown before and after drought recovery. As shown in FIG. 6, transgenic Arabidopsis thaliana plants overexpressing of FMO RC15 GS-OX5 recover from drought stress better than wild-type plants.

### Example 9

### Overexpression of FMO RC15 GS-OX5 Results in Increased Biomass

In order to determine the plant biomass analysis, Arabidopsis plants were grown for three (3) weeks under short day (10 hours light, 14 hours dark, 21° C. light and 20° C. at night, 65% humidity) conditions. Fresh weight from individual rosettes was obtained, Col-0 (n=10) and RCI5-OE (ES 2347399B1) (FMO3X and FMO8X genotypes) two weeks after sowing (n=10). Seeds yield of fully grown plants that were grown for 3 weeks under short day conditions and then transferred for 3 additional weeks to long day conditions was recorded. Seeds were harvested 4 weeks later from individual plants (n=10).

As shown in Table 3 below, overexpression of FMO RC15 GS-OX5 in Arabidopsis thaliana results in a biomass mean weight increase in plants grown under no stress conditions. The increase in mean weight was significantly greater in FMO8X lines, when the level of RC15 expression was greater compared to the level of expression in wild-type. Column one shows the genotype, column two shows the number of plants (N), column three shows plant biomass evaluated as average weight (in grams) plus or minus the standard error (S.E.), and column four shows the ANOVA P-value.

TABLE 3
Biomass mean weight in FMO GS-OX5 transgenic Arabidopsis plants

| Genotype | N | Biomass Mean Weight Value ± S.E | ANOVA P-value |
|---|---|---|---|
| Col-0 | 10 | 2.0637 ± 0.2240 | |
| FMO3X | 10 | 1.9199 ± 0.1383 | 0.5917 |
| FMO8X | 10 | 2.5815 ± 0.1191 | 0.023* |

### Example 10

### Overexpression of FMO RC15 GS-OX5 Results in Increased Seed Yield as Measured by Seed Weight

As shown in Table 4 below, the seed mean weight also increased with increasing levels of FMO RC15 GS-OX5, being greater in the FMO8X line. Plant seed yield was evaluated for three different groups of seeds and siliques from Arabidopsis plants grown under no stress conditions. Column one shows the genotype, column two shows the number of plants (N), column three shows the total seed mean weight in mg plus or minus the standard error (S.E.), and column four shows the ANOVA P-value.

TABLE 4
Seed mean weight in FMO GS-OX5 transgenic Arabidopsis plants

| Genotype | N | Seed Mean Weight Value ± S.E | ANOVA P-value |
|---|---|---|---|
| Col-0 | 10 | 522.8 ± 22.64 | |
| FMO3X | 10 | 495.1 ± 37.22 | 0.5330 |
| FMO8X | 10 | 546.3 ± 35.09 | 0.5806 |

### Example 11

### Overexpression of FMO GS-OX5 Increases Plant Survival Under Drought Conditions

As shown in Table 5 below, transgenic plants overexpressing FMO RC15 GS-OX5 and wild-type plants treated with TMAO di-hydrate had a significantly higher fitness value than non-transgenic Arabidopsis plants under drought conditions. Transgenic FMO3X and FMO8X genotypes and wild type Col-0 seeds of Arabidopsis thaliana were sown, grown and treated as described above. For the control group of both wild-type and transgenic plants, six week old plants were irrigated with 40 ml of water twice in the week, while “drought” treated plants of both wild-type and transgenic plants were not irrigated until all the plants were wilted.

After the first cycle of wilting wild type plants were sprayed with 1 g/L TMAO di-hydrate to determine if the wilted wild type plants could recover and perform as well as the transgenic plants in the following cycles of wilting. Fitness values were assigned using the following criteria: 0: Dead plant; 1: Critically damaged plant symptoms; 2: Moderate damaged plant symptoms; 3: Slightly damaged plant symptoms; and 4: Healthy plant. Column one shows the genotype of the plant, column two shows the number of plants (N), column three shows the mean fitness value plus or minus the standard error (S.E.), and column four shows the ANOVA P-value.

TABLE 5
Mean fitness value in FMO GS-OX5 transgenic Arabidopsis plants

| Genotype | N | Mean Fitness Value ± S.E. | ANOVA P-value |
|---|---|---|---|
| Col-0 | 36 | 1.14 ± 0.17 | — |
| Col-0 + 1 g/L | 36 | 1.83 ± 0.21 | 0.0129* |
| Sprayed TMAO | | | |
| di-hydrate solution | | | |
| FMO3X | 36 | 2.67 ± 0.08 | 0.0000* |
| FMO8X | 36 | 2.64 ± 0.08 | 0.0000* |

### Example 12

### Overexpression of FMO GS-OX5 Increases Plant Fitness Under Limited Water Conditions

As shown in Table 6 below, overexpression of FMO GS-OX5 increases plant survival in Arabidopsis under limited water irrigation. Control plants (six weeks old) were irrigated with 40 ml of water twice in the week, while “limited water irrigation” treated plants were irrigated with 30 ml of water once a week. Transgenic (FMO3X and FMO8X genotypes) and wild type (Col-0) seeds of Arabidopsis thaliana were sown, grown and treated as described herein. The fitness value increased with increasing levels of FMO RC15 GS-OX5 expression, being greater in FMO8X lines. Fitness values were assigned using the following criteria: 0: Dead plant; 1: Critically damaged plant symptoms; 2: Moderate damaged plant symptoms; 3: Slightly damaged plant symptoms; 4: Healthy plant. Column one shows the genotype of the plant, column two shows the number of plants (N), column three shows the mean fitness value plus or minus the standard error (S.E.), and column four shows the ANOVA P-value. As shown in Table 6, the transgenic plants had a significantly higher fitness value than wild-type plants.

TABLE 6
Average fitness value for FMO GS-OX5 transgenic Arabidopsis plants

| Genotype | N | Mean Fitness Value ± S.E | ANOVA P-value |
|---|---|---|---|
| Col-0 | 60 | 1.75 ± 0.09 | — |
| FMO3X | 60 | 2.533 ± 0.09 | 0.0000* |
| FMO8X | 60 | 3.066 ± 0.09 | 0.0000* |

### Example 13

### Overexpression of FMO GS-OX5 in Arabidopsis Alters Gene Expression

Genome-wide transcriptome analysis of Arabidopsis transgenic plants overexpressing FMO GS-OX5 (RCI5-OE.FMO8X) and having increased TMAO levels shows that RC15 transgenic plants have altered gene expression. Wild-type (Col) and RCI5-OE (FMO8X) 12-day-old plants, grown in vitro in MS medium supplemented with 1% sucrose, were collected for RNA isolation. Total RNA was extracted using the RNeasy Mini Kit (Qiagen). Preparation of RNA-seq libraries and subsequent sequencing (Highseq 50SE) was performed by BGI (Shenzhen, China). The raw reads were aligned to the Arabidopsis genome (TAIR10, please see the Arabidopsis Information website, TAIR, and Ohio State University) by using TopHat program. The assembling of the reads and the calculation of transcript abundance were performed by Cufflinks. Transcripts that were differentially expressed (Pval<0.05 and FDR<0.001) in WT and RCI5-OE (FMO8X) were identified by Cuffdiff, a part of the Cufflinks package.

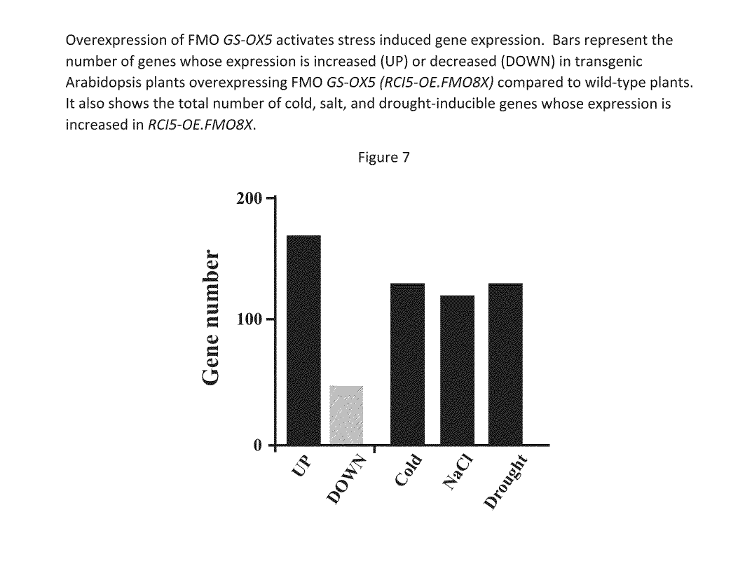

As shown in FIG. 7, transgenic plants had an increasing accumulation of a significant number of mRNAs (>150). Moreover, thirteen of these genes, including SUS4 and DIN10, which encode key enzymes in sucrose and raffinose biosynthesis, respectively, have been shown to be involved in drought tolerance (Maruyama et al., Plant Physiology 150: 1972, 2009).

### Example 14

### Phylogenetic Tree Based on FMO Protein Similarities

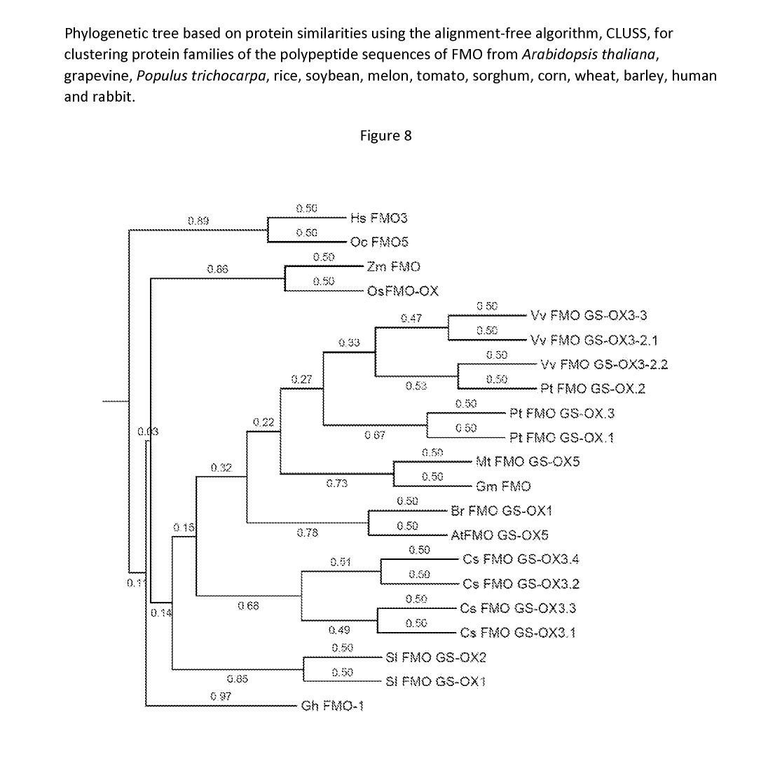

As discussed below, FIG. 8 provides a phylogenetic tree of the polypeptide sequences listed above of FMO proteins from Arabidopsis thaliana, grapevine, Populus trichocarpa, rice, soybean, melon, tomato, sorghum, corn, wheat, barley, human and rabbit.

Genes with high identity to FMO GS-OX5 mediate similar functions. Amino acid and nucleic acid sequences can be aligned using methods known in the art. As shown in FIG. 8 FMO proteins may have 40% or more identity, including but not limited to at least 50%, at least 60%, at least 70%, at least 80%, at least 85%, at least 90%, at least 95%, at least 97%, at least 98%, at least 99% or more identity, in comparison with the respective FMO RC15 GS-OX5 sequence of Arabidopsis (At1g12140) (SEQ ID NO: 1) [cDNA sequence with UTR] or the protein sequence SEQ ID NO.: 2). The genes with the highest homologies to At1g12140 from Solanum lycopersicum SlFMO GS-OX1 (Solyc06g060610) (SEQ ID GS-OX3-1 (SEQ ID NO: 21 and SEQ ID NO: 22) (LOC100242032), VvFMO GS-OX3 (LOC100255688) (SEQ ID NO: 19 SEQ ID NO: 20), VvFMO GS-OX3-3 (LOC100255688) (SEQ ID NO: 17 and SEQ ID NO: 18), Populus trichocarpa PtFMO-GS-OX3 (XM_002329873.1) (SEQ ID NO: 27 and SEQ ID NO: 28), PtFMO GS-OX2 (XM_002318967.1) (SEQ ID NO: 29 and SEQ ID NO: 30), PtFMO GS-OX1 (XP002318210.1), Oryza sativa OsFMO-OX (Os10g40570.1) (SEQ ID NO: 15 and SEQ ID NO: 16), Glycine max GmFMO (Glyma11g03390.1) (SEQ ID NO: 33 and SEQ ID NO: 34), Cucumus sativus CsFMO GS-OX3-1 (LOC101227975) (SEQ ID NO: 11 and SEQ ID NO: 12), CsFMO GS-OX3-2 (LOC101220079) (SEQ ID NO: 9 and SEQ ID NO: 10), CsFMO GS-OX3-3 (LOC101220318) (SEQ ID NO: 7 and SEQ ID NO: 8), CsFMO GS-OX3-4 (LOC101212991) (SEQ ID NO: 5 and SEQ ID NO: 6), Brassica rapa subsp. pekinensis BrFMO GS-OX1 (FJ376070.1), Medicago truncatula MtFMO GS-OX5 (MTR_5g012130) (SEQ ID NO: 13 and SEQ ID NO: 14), Zea mays Zm FMO (GRMZM2G089121_P01) (SEQ ID NO: 25 and SEQ ID NO: 26), Gossypium hirsutum GhFMO-1 (DQ122185.1) SEQ ID NO: 23 and SEQ ID NO: 24) Homo sapiens HsFMO-3 (NP_001002294.1) (SEQ ID NO: 39 and SEQ ID NO: 40) and Oryctolagus cuniculus OcFMO-5 (NP_001075714.1) SEQ ID NO: 41 and SEQ ID NO: 42) probably exert similar functions in the plant or photosynthetic organism as FMO GS-OX5 polypeptide from Arabidopsis (AtFMO GS-OX5).

As shown in FIG. 8, the equivalent expression of FMO proteins may be expected for sequences having 40% or more identity, including but not limited to at least 50%, at least 60%, at least 70%, at least 80%, at least 85%, at least 90%, at least 95%, at least 97%, at least 98%, at least 99% or more identity, in comparison with other FMO sequences such as the respective FMO GS-OX5 sequence of Arabidopsis.

### Biological Material and Growth Conditions for Greenhouse Drought or Limited Water Experiments

For each drought or limited water experiment 480 seeds (of either pepper, barley, tomato, cucumber or corn) were sown, producing 384 plants in 512 cm³ pots (4 plants per pot). Plants were grown under chamber conditions at 21° C. for 3 weeks. Then, the plants were moved to a greenhouse, where average temperature was 25° C. to 28° C. Spray and irrigation treatments as described herein were done when the plants had two extended leaves and the next pair of leaves were coming up.

Treatments: Twelve pots (containing 48 plants) were irrigated with 40 ml of either: water, 0.1 g/L TMAO di-hydrate solution, 1.0 g/L TMAO di-hydrate solution, or 5.5 g/L TMAO di-hydrate solution. Another set of 12 pots containing 48 plants were sprayed with 40 ml of either water (3.3 ml in average per pot), a solution containing 0.1 g/L TMAO di-hydrate solution, 1.0 g/L TMAO di-hydrate, or 5 g/L, or 10 g/L TMAO di-hydrate. Further sets of 12 pots containing 48 plants were both sprayed with each initial solution TMAO di-hydrate solution and further irrigated with the same TMAO di-hydrate solutions used in the control water sprayed plants. All pots were also watered with 40 ml of water. The sprayed plants were watered with the same volume of water as the “irrigated plants). The pots were located on plastic glass to maintain constant moisture and to avoid liquid spillage during watering. Trays containing the pots were located on greenhouse tables. The distribution of the trays on the table and the position on the pots in the tray was changed every week to avoid position effects.

### Extreme Drought Conditions

After the treatments described above, the plants were not watered until the pots completely lost their moisture, taking about 4 to 8 days depending on the season, at which point the plants were extremely wilted for the extreme drought experiments. The plants were then watered once with solutions containing the different amounts of TMAO di-hydrate (0.1 g/L, 1.0 g/L, 5 g/L, or 10 g/L) or just water, after which the plants were left to lose their moisture completely again for three consecutive cycles of watering after wilting. For the “extreme drought” experiments plants were allowed to wilt severely before watering and then the plant survival rate was recorded and analyzed.

### Limited Water Conditions

After the treatments described above, for the “limited water” experiments plants were watered with 20 ml of water or solution instead of 40 ml when the first plants started to wilt. The stem length was recorded as analyzed for the limited water experiments in which the plants are watered with 50-30% of the water that the plant requires.

### Example 15

### Tomato Plants Irrigated or Sprayed with TMAO Di-Hydrate Recover Better from Drought Stress than Plants Irrigated with Water

TMAO di-hydrate applied exogenously, which increases the endogenous content of TMAO as if the plants were overexpressing an FMO protein at least 4 times, increases tomato plant survival under extreme drought conditions, where plants were allowed to fully wilt after three water-wilt cycles. Moneymaker tomato seeds were sown, grown and treated as described above. No statistical differences between modes of application (sprayed or TMAO di-hydrate watered) were observed on this experiment.

As shown in Table 7 below, plants sprayed with 5 g/L TMAO di-hydrate and then irrigated with water resulted in the greatest plant survival rate, at 74.2%. At higher test rates, both treatments showed a clear increase of survival rate when compared with untreated plants.

TABLE 7
Average survival rate and ANOVA analysis for TMAO
di-hydrate treated tomato plants under drought conditions

| | | INITIAL SPRAY | | SURVIVAL | ANOVA |
|---|---|---|---|---|---|
| IRRIGATION | N | TREATMENT | | RATE (%) | P-value |
| ALL REGIMES | 384 | WATER | | 12.5 ± 4.1 | 0.0000 |
| | | 0.1 | g/L TMAO | 12.5 ± 4.1 | |
| | | 1 | g/L TMAO | 37.5 ± 4.1 | |
| | | 5 | g/L TMAO | 56.6 ± 4.1 | |
| WATER | 96 | WATER | | 16.6 ± 9.1 | 0.0000 |
| | | 0.1 | g/L TMAO | 29.1 ± 9.1 | |
| | | 1 | g/L TMAO | 62.5 ± 9.1 | |
| | | 5 | g/L TMAO | 74.2 ± 9.1 | |
| 0.1 g/L TMAO | 96 | WATER | | 16.6 ± 8.5 | 0.0000 |
| | | 0.1 | g/L TMAO | 12.5 ± 8.5 | |
| | | 1 | g/L TMAO | 41.6 ± 8.5 | |
| | | 5 | g/L TMAO | 68.9 ± 8.5 | |
| 1 g/L TMAO | 96 | WATER | | 4.1 ± 7.5 | 0.0013 |
| | | 0.1 | g/L TMAO | 0.0 ± 7.5 | |
| | | 1 | g/L TMAO | 29.1 ± 7.5 | |
| | | 5 | g/L TMAO | 33.3 ± 7.5 | |
| 5 g/L TMAO | 96 | WATER | | 8.3 ± 8.0 | 0.0015 |
| | | 0.1 | g/L TMAO | 12.5 ± 8.0 | |
| | | 1 | g/L TMAO | 16.6 ± 8.0 | |
| | | 5 | g/L TMAO | 50.0 ± 8.0 | |

In rows 1-4 the spray treatments are compared independently from the irrigation treatments. The survival rate after drought significantly increases with the concentration of the TMAO di-hydrate spray being the lowest in row 1 without TMAO di-hydrate (12.5%) and the highest in row 4 with 5 g/L of TMAO (56.6%). In rows 5-8 the spray treatments are compared when the plants are irrigated only with water. Survival rate after drought significantly increases with the concentration of the TMAO di-hydrate spray being the lowest in row 5 without TMAO di-hydrate (16.6%) and the highest in row 8 with 5 g/L of TMAO di-hydrate (74.2%).

In rows 9-12 the spray treatments are compared when the plants are irrigated with 0.1 g/L of TMAO di-hydrate. Survival rate after drought significantly increases with the highest concentrations of the TMAO spray being the lowest in rows 9 and 10, without TMAO di-hydrate (16.6%) and 0.1 g/L TMAO di-hydrate spray (12.5%) respectively, and the highest in row 12 with 5 g/L of TMAO di-hydrate (68.9%). In rows 13-16 the spray treatments are compared when the plants are irrigated with 1 g/L of TMAO di-hydrate. Survival rate after drought also significantly increases with the highest concentrations of the TMAO di-hydrate spray being the lowest in rows 13 and 14, without TMAO di-hydrate (4.1%) and 0.1 g/L TMAO di-hydrate spray (0%) respectively, and the highest in row 16 with 5 g/L of TMAO di-hydrate (33.3%) which is consistent with the fact that higher levels of FMO overexpression increases drought tolerance because the endogenous levels of TMAO are proportional to the level of overexpression. Increasing the TMAO di-hydrate irrigation treatment to 5 g/L (rows 17-20) improves the survival rates when compared to low dose irrigation treatments combined with spray treatments. Combining the highest doses of spray 5 g/L and irrigation 5 g/L renders a survival rate of 50% (row 20).

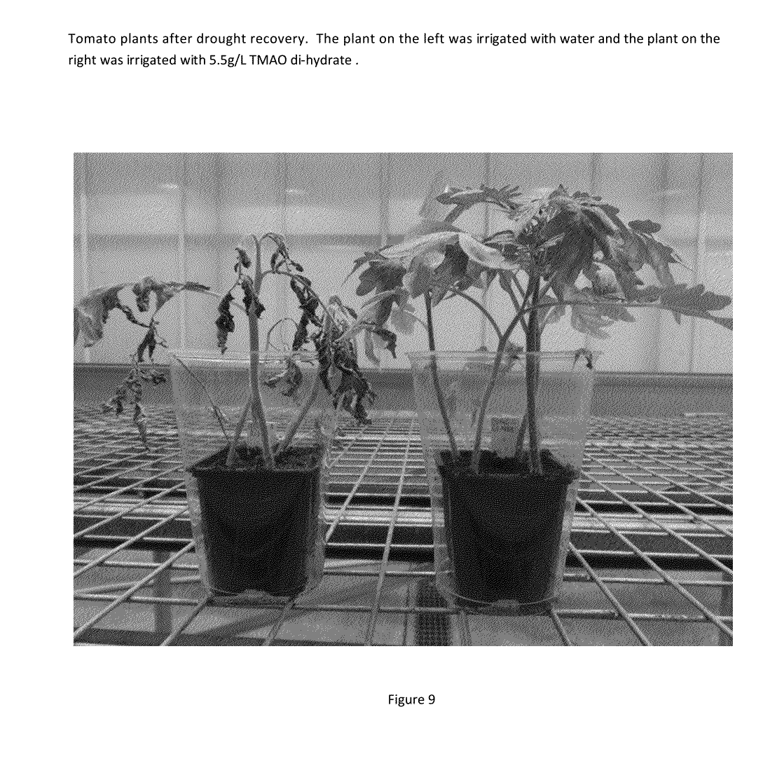

Additionally, TMAO di-hydrate treated plants appeared extremely healthy compared to untreated control plants (FIG. 9). As shown in FIG. 9, 5.5 g/L TMAO di-hydrate was used to irrigate the plant on the right-hand side, whereas on the left-hand side the control plant was irrigated with water. The plants are shown 24 hours after drought recovery.

### Example 16

### Tomato Plants Irrigated with TMAO Di-Hydrate have Longer Stem Size Compared to Plants Irrigated with Water

TMAO di-hydrate applied exogenously which increases the endogenous content of TMAO as if the plants were overexpressing an FMO protein at least 4 times increases plant stem size in tomato under limited water irrigation. ‘Moneymaker’ tomato seeds were sown, grown and treated as described. Both spray and irrigation treatments with TMAO di-hydrate increased significantly plant stem size.

TABLE 8
Average stem size and ANOVA analysis for TMAO and water
irrigated tomato plants under limited water growing conditions

| INITIAL | | | AVERAGE STEM | ANOVA |
|---|---|---|---|---|
| TREATMEN | N | IRRIGATIONS | SIZE (cm) | P-value |
| WATER | 94 | WATER | 10.57 ± 0.56 | 0.0000 |
| | | 1 g/L TMAO | 12.97 ± 0.55 | |
| 0.1 g/L TMAO | 93 | WATER | 11.06 ± 0.55 | 0.1034 |
| | | 1 g/L TMAO | 12.32 ± 0.56 | |
| 1 g/L TMAO | 96 | WATER | 11.59 ± 0.55 | 0.0000 |
| | | 1 g/L TMAO | 13.77 ± 0.55 | |
| 5 g/L TMAO | 92 | WATER | 14.2 ± 0.56 | 0.7230 |
| | | 1 g/L TMAO | 14.6 ± 0.55 | |

Table 8 shows that TMAO di-hydrate can be applied exogenously by spray and watering before the drought stress occurs increasing the stem biomass in the Solanaceae family, under limited drought stress conditions. In rows 1-2 the irrigation treatments are compared independently from the spray treatments. The stem length significantly increases after limited irrigation with 1 g/L TMAO di-hydrate spray being the shortest in row 1 without TMAO di-hydrate (10.57 cm) and the longest in row 2 with 1 g/L of TMAO di-hydrate spray (12.97 cm). In rows 1, 3, 5 and 7 the spray treatments are compared when the plants are irrigated only with water. Stem length after limited water irrigation significantly increases with the concentration of the TMAO di-hydrate spray being the shortest in row 1 without TMAO di-hydrate (10.57 cm) and the longest in row 7 with 5 g/L of TMAO di-hydrate (14.2 cm). In rows 2, 4, 6 and 8 the spray treatments are compared when the plants are irrigated with 1 g/L of TMAO di-hydrate. Again stem length significantly increases after limited water irrigation with the increasing concentrations of the TMAO di-hydrate spray being the shortest in row 2, without TMAO di-hydrate spray (12.97 cm) and the longest in row 8 when both treatments are combined with 5 g/L of TMAO spray treatment and 1 g/L irrigation treatment (14.6 cm).

### Example 17

### Tomato Plants Irrigated with TMAO Di-Hydrate have Larger Fruit Compared to Plants Irrigated with Water

TMAO di-hydrate applied exogenously which increases the endogenous content of TMAO as if the plants were overexpressing an FMO protein at least 4 times increases plant production in tomato under limited water irrigation. ‘Rio Grande’ tomato seeds were sown, grown and treated as described. Spray treatments with 1 g/L TMAO di-hydrate increased both fruit size and fruit production.

TABLE 9
Average fruit production and ANOVA analysis for TMAO di-hydrate
spray treated tomato plants under limited water growing conditions.

| | | INITIAL | AVERAGE WEIGHT | ANOVA |
|---|---|---|---|---|
| IRRIGATION | N | TREATMENT | (grams/fruit) | P-value |
| 100% WATER | 36 | WATER | 73.85 ± 17.84 | — |
| 30% WATER | 36 | WATER | 52.9 ± 17.28 | 0.4243 |
| 30% WATER | 36 | 1 g/L TMAO | 76.73 ± 17.67 | 0.3406 |

Table 9 shows that TMAO di-hydrate can be applied exogenously by spray which increases the endogenous content of TMAO as if the plants were overexpressing an FMO protein at least 4 times before the drought stress occurs increasing the average fruit production (i.e., increases both the weight of the fruit and the amount of fruit) in the Solanaceae family, under limited drought stress conditions. In row 2 it is shown that 30% water irrigation significantly lowers plant production (52.9 g/fruit) when compared with plants in row 1 under normal water irrigation (73.85 g/fruit). However, as shown in row 3, spray treatment with 1 g/L of TMAO di-hydrate applied exogenously every 4 weeks restores plant production with an increase of fruit production of 45% even under limited water irrigation (76.73 g/fruit) over the untreated plants with a 30% irrigation.

### Example 18

### Pepper Plants Irrigated with TMAO Di-Hydrate Recover Better from Drought Stress than Plants Irrigated with Water

TMAO di-hydrate applied exogenously, which increases the endogenous content of TMAO as if the plants were overexpressing an FMO protein at least 4 times increases plant survival in pepper plants under extreme drought conditions. ‘Murano’ pepper seeds were sown, grown and treated as described above. 0.1 g/L TMAO di-hydrate irrigation combined with 10 g/L TMAO di-hydrate sprayed resulted in 83.3% of plant survival while 100% plant survival rate was observed when plants were sprayed with 0.1 g/L or 1 g/L and irrigated with 5 g/L TMAO di-hydrate.

TABLE 10
Average survival rate and ANOVA analysis for TMAO
di-hydrate treated pepper plants under drought growing conditions

| | | INITIAL | | | |
|---|---|---|---|---|---|
| | | SPRAY | | SURVIVAL | ANOVA |
| IRRIGATION | N | TREATMENT | | RATE (%) | P-value |
| ALL | 384 | WATER | | 42.7 ± 3.6 | 0.0000 |
| REGIMES | | 0.1 | g/L TMAO | 51.0 ± 3.6 | |
| | | 1 | g/L TMAO | 62.5 ± 3.6 | |
| | | 10 | g/L TMAO | 71.8 ± 3.6 | |
| WATER | 96 | WATER | | 45.8 ± 8.1 | 0.0025 |
| | | 0.1 | g/L TMAO | 37.5 ± 8.1 | |
| | | 1 | g/L TMAO | 62.5 ± 8.1 | |
| | | 10 | g/L TMAO | 79.1 ± 8.1 | |
| 0.1 g/L TMAO | 96 | WATER | | 29.1 ± 8.3 | 0.0000 |
| | | 0.1 | g/L TMAO | 33.3 ± 8.3 | |
| | | 1 | g/L TMAO | 54.1 ± 8.3 | |
| | | 10 | g/L TMAO | 83.3 ± 8.3 | |
| 1 g/L TMAO | 96 | WATER | | 0.0 ± 7.7 | 0.0028 |
| | | 0.1 | g/L TMAO | 33.3 ± 7.7 | |
| | | 1 | g/L TMAO | 33.3 ± 7.7 | |
| | | 10 | g/L TMAO | 37.5 ± 7.7 | |
| 5 g/L TMAO | 96 | WATER | | 95.8 ± 3.8 | 0.0812 |
| | | 0.1 | g/L TMAO | 100 ± 3.8 | |
| | | 1 | g/L TMAO | 100 ± 3.8 | |
| | | 10 | g/L TMAO | 87.5 ± 3.8 | |

Table 10 shows that TMAO di-hydrate can be applied exogenously by spray and/or irrigation to increase the endogenous content of TMAO as if the plants were overexpressing an FMO protein at least 4 times before drought stress occurs increasing the plant survival rate under extreme drought stress conditions in a vegetable crop species. In rows 1-4 the spray treatments are compared independently from the irrigation treatments. The survival rate after drought significantly increases with the concentration of the TMAO di-hydrate spray being the lowest in row 1 without TMAO di-hydrate (42.7%) and the highest in row 4 with 10 g/L of TMAO di-hydrate (71.8%), which is consistent with the fact that higher levels of FMO overexpression increases drought tolerance because the endogenous levels of TMAO are proportional to the level of overexpression. In rows 5-8 the spray treatments are compared when the plants are irrigated only with water. Survival rate after drought significantly increases with the concentration of the TMAO di-hydrate spray being the lowest in row 5 without TMAO di-hydrate (45.8%) and the highest in row 8 with 10 g/L of TMAO di-hydrate (79.1%). In rows 9-12 the spray treatments are compared when the plants are irrigated with 0.1 g/L of TMAO di-hydrate. Survival rate after drought significantly increases with the concentration of the TMAO di-hydrate spray being the lowest in row 9 without TMAO di-hydrate (29.1%) and the highest in row 12 with 10 g/L of TMAO di-hydrate (83.3%). In rows 13-16 the spray treatments are compared when the plants are irrigated with 1 g/L of TMAO di-hydrate. Survival rate after drought also significantly increases with the concentration of the TMAO spray being the lowest in row 13 without TMAO di-hydrate (0%) and the highest in row 16 with 10 g/L of TMAO di-hydrate (37.5%). The best results are achieved when plants are irrigated with TMAO di-hydrate at 5 g/L (rows 17-20). Even without spray treatment the survival rate is 95.8% (row 17), which increases up to 100% survival with 0.1 g/L and 1 g/L spray treatments (rows 18-19).

### Example 19

### Cucumber Plants Irrigated with TMAO Di-Hydrate Recover Better from Drought Stress than Plants Irrigated with Water

TMAO di-hydrate applied exogenously which increases the endogenous content of TMAO as if the plants were overexpressing an FMO protein at least 4 times increases plant survival in cucumber under extreme drought conditions. ‘Marketer’ cucumber seeds were sown, grown and treated as described above.

TABLE 11
Average survival rate and ANOVA analysis for TMAO di-hydrate
treated cucumber plants under drought growing conditions

| | | INITIAL | | | |
|---|---|---|---|---|---|
| | | SPRAY | | SURVIVAL | ANOVA |
| IRRIGATION | N | TREATMENT | | RATE (%) | P-value |
| ALL | 384 | WATER | | 66.6 ± 3.4 | 0.0000 |
| REGIMES | | 0.1 | g/L TMAO | 80.1 ± 3.4 | |
| | | 1 | g/L TMAO | 92.7 ± 3.4 | |
| | | 5 | g/L TMAO | 94.7 ± 3.4 | |
| WATER | 96 | WATER | | 54.1 ± 7.2 | 0.0004 |
| | | 0.1 | g/L TMAO | 83.3 ± 7.2 | |
| | | 1 | g/L TMAO | 91.6 ± 7.2 | |
| | | 5 | g/L TMAO | 95.8 ± 7.2 | |

| | | | | | |
|---|---|---|---|---|---|
| 0.1 | g/L TMAO | 96 | WATER | 45.8 ± 7.4 | 0.0000 |

| | | |
|---|---|---|
| 0.1 | g/L TMAO | 82.9 ± 7.4 |
| 1 | g/L TMAO | 91.6 ± 7.4 |
| 5 | g/L TMAO | 95.8 ± 7.4 |

| | | | | | |
|---|---|---|---|---|---|
| 1 | g/L TMAO | 96 | WATER | 87.5 ± 5.9 | 0.0028 |

| | | |
|---|---|---|
| 0.1 | g/L TMAO | 91.6 ± 5.9 |
| 1 | g/L TMAO | 91.6 ± 5.9 |
| 5 | g/L TMAO | 91.6 ± 5.9 |

| | | | | | |
|---|---|---|---|---|---|
| 5 | g/L TMAO | 96 | WATER | 66.6 ± 7.2 | 0.0812 |

| | | |
|---|---|---|
| 0.1 | g/L TMAO | 75.0 ± 7.2 |
| 1 | g/L TMAO | 95.8 ± 7.2 |
| 5 | g/L TMAO | 95.8 ± 7.2 |

Table 11 shows that TMAO di-hydrate can be applied exogenously by spray and/or watering before the drought stress occurs increasing the plant survival rate in the Cucurbitaceae family, under extreme drought stress conditions, where plants were allowed to fully wilt after three water-wilt cycles. In rows 1-4 the spray treatments are compared independently from the irrigation treatments. The survival rate after drought significantly increases with the concentration of the TMAO di-hydrate spray being the lowest in row 1 without TMAO di-hydrate (66.6%) and the highest in row 4 with 5 g/L of TMAO (94.7%). In rows 5-8 the spray treatments are compared when the plants are irrigated only with water. Survival rate after drought significantly increases with the concentration of the TMAO di-hydrate spray being the lowest in row 5 without TMAO di-hydrate (54.1%) and the highest in row 8 with 5 g/L of TMAO di-hydrate (95.8%). In rows 9-12 the spray treatments are compared when the plants are irrigated with 0.1 g/L of TMAO di-hydrate. Survival rate after drought significantly increases with the concentration of the TMAO di-hydrate spray being the lowest in row 9 without TMAO di-hydrate (45.8%) and the highest in row 12 with 5 g/L of TMAO (95.8%). In rows 13-16 the spray treatments are compared when the plants are irrigated with 1 g/L of TMAO di-hydrate. Survival rate after drought also significantly increases with the any of the TMAO di-hydrate spray treatments being the lowest in row 13 without TMAO (87.5%) and higher in rows 14-16 with 0.1, 1 or 5 g/L of TMAO di-hydrate giving the same 91.6% survival rate. Plants irrigated with TMAO di-hydrate at 5 g/L (rows 17-20) showed the greatest survival rate. Even without spray treatment the survival rate is 66.6% (row 17), which increases up to 95.8% survival with 5 g/L spray treatment (row 20)

### Strawberries, Leek, Lettuce, Broccoli, Celery or Kohlrabi

In order to determine the plant yield productivity under normal conditions, ‘Sabrina’, ‘Candonga’ and ‘Fortuna’ strawberry varieties, leek, lettuce, “Iceberg” variety, broccoli “Parthenon” variety, celery or kohlrabi plants, were grown under standard production conditions and 120 plants of each variety per treatment (where the treatment was a control comprising standard watering or 1 g/L of TMAO di-hydrate spray every four weeks) were analyzed. Plants were located in four (4) different positions for each group of 30 plants from the same treatment. Fruits, leaves or roots were harvested from individual plants and total weight was determined for each plant.

### Example 20

### Exogenous Application of TMAO Di-Hydrate does not have Trade-Offs in Strawberry

Fruit yield was determined in ‘Sabrina’, ‘Candonga’ and ‘Fortuna’ strawberry plants treated with 1 g/l of TMAO di-hydrate or water as described above in order to evaluate the trade-off costs of the treatment with no drought stress. However, no significant difference was observed in the fruit production which was always slightly higher in the TMAO di-hydrate treated plants.

TABLE 12
Strawberry fruit production after TMAO di-hydrate spray treatments
every 4 weeks for 3 months

| Crop | % Control (2013) | % Control (2014) |
|---|---|---|
| Sabrina | 106 | 115 |
| Candonga | 102 | 106 |
| Fortuna | 101 | 105 |
| Total | 105 | 111 |

### Example 21

### TMAO Di-Hydrate Spray Treatment does not Negatively Affect Yield in Leek, Lettuce, Broccoli, Celery, Garlic, or Kohlrabi Crops

Exogenous application of TMAO di-hydrate which increases the endogenous content of TMAO as if the plants were overexpressing an FMO protein at least 4 times does not have trade-offs in leek, lettuce, broccoli, celery or kohlrabi. Root or leaves yield was determined in the plants treated with 1 g/l of TMAO di-hydrate or water as described above in order to evaluate the trade-off costs of the treatment with no drought stress. However, no significant difference was observed in the yield production which was in most cases slightly higher in the TMAO di-hydrate treated plants.

TABLE 13
Yield production after TMAO di-hydrate spray treatments every
4 weeks for 3 months

| Crop | % Control |
|---|---|
| Leek | 102 |
| Lettuce | 112 |
| Broccoli | 120 |
| Celery | 100 |
| Kohlrabi | 103 |
| Garlic | 109 |

Table 13 shows that TMAO di-hydrate can be applied exogenously at least 3 times for three months without a fitness cost. In row 1 the total production weight of leek plants treated with TMAO di-hydrate produced 102% when compared with water treated controls, in row 2 the total production weight of lettuce plants treated with TMAO di-hydrate produced 112% when compared with controls, in row 3 the total production weight of broccoli plants treated with TMAO di-hydrate produce 120% when compared with controls, while in row 4 the total production weight of the celery plants treated with TMAO di-hydrate produce the same as water treated controls, in row 5 kohlrabi plants produced 103% when compared with water treated controls, and finally in row 6 garlic plants produced 109% when compared with water treated controls.

### Example 22

### Broccoli Plants Treated with TMAO Di-Hydrate have Increased Inflorescence Production

TMAO di-hydrate applied exogenously which increases the endogenous content of TMAO as if the plants were overexpressing an FMO protein at least 4 times increases plant production in broccoli under limited water irrigation. ‘Parthenon’ broccoli seeds were sown, grown and treated as described above. Spray and irrigation treatments with 1 g/L TMAO di-hydrate increased plant production, as measured by the average weight of the crown plus stems in grams.

TABLE 14
Average inflorescence production and ANOVA analysis for
TMAO di-hydrate spray treated broccoli plants under limited water
growing conditions.

| | | | AVERAGE | | |
|---|---|---|---|---|---|
| | | | WEIGHT | ANOVA | |
| | | TREAT- | (grams/ | P- | % |
| IRRIGATION | N | MENT | inflorescence) | value | Control |
| 100% | 36 | WATER | 202.8 ± 17.5 | — | 250 |
| WATER | | | | | |
| 30% | 36 | WATER | 80.5 ± 8.9 | 0.4243 | — |
| WATER | | | | | |
| 30% | 36 | 1 g/L TMAO | 87.3 ± 6.7 | 0.3406 | 108 |
| WATER | | spray | | | |
| 30% | 36 | 1 g/L TMAO | 85.2 ± 4.6 | 0.3406 | 106 |
| WATER | | irrigation | | | |

Table 14 shows that TMAO di-hydrate can be applied exogenously by spray to increase the production of broccoli under limited drought stress conditions. In row 2 it is shown that 30% water irrigation significantly lowers plant production (80.5 g/plant) when compared with plants in row 1 under normal water irrigation (202.8 g/plant). However, as shown in rows 3 and 4, spray or irrigation treatment with 1 g/L of TMAO di-hydrate applied exogenously every 4 weeks partially restores plant production with an increase of inflorescence production of 8% or 6% respectively even under limited water irrigation (87.3 g/plant and 85.2 g/plant) over the untreated plants with a 30% irrigation.

### Corn, Barley and Sunflower Field Trials

In order to determine the drought or drought stress tolerance after seed treatments with TMAO di-hydrate and germination in the presence of TMAO di-hydrate, barley “Hispanic” seeds, corn “FAO700” seeds, and “Sambro” sunflower seeds were surface sterilized for 3 minutes in ethanol 70%, then rinsed twice and finally included in a pre-treatment solution of 1 g/L TMAO di-hydrate solution (or just water) under shaking for 3 hours at a dose of 1 litre per Kg of seeds. Then, the seeds were sown in randomized plots of 10 sqm in a surface of 2.000 sqm. Chlorophyll content was measured 1 month before harvest. In corn irrigation was applied in half of the plots while the other half only received an initial establishment watering. The barley plots received 200 l of rain per m² through the growing season. Some of the plots received a second spray treatment with 1 g/liter of TMAO. TMAO content was determined by harvesting 3 leaves per treatment and freezing them in liquid nitrogen before NMR determination. At least 3 independent plants were treated per experiment.

### Example 23

### Barley Plants Irrigated with TMAO have Greater Average Dry Weight than Plants Irrigated with Water

TMAO di-hydrate applied exogenously which increases the endogenous content of TMAO as if the plants were overexpressing an FMO protein at least 4 times increases plant survival and biomass in barley under limited water irrigation. ‘Bomi’ barley seeds were sown, grown and treated as described. Average dry weight includes the whole plant minus the stems.

TABLE 15
Average dry weight ± S.E. and ANOVA analysis for TMAO di-hydrate
and water irrigated barley plants under drought growing conditions

| INITIAL | | | AVERAGE | ANOVA |
|---|---|---|---|---|
| TREATMENT | N | IRRIGATIONS | DRY | P-value |
| CONTROL | 10 | WATER | 1017.7 ± 66.13 | — |
| 1 G/L | 12 | WATER | 1205.4 ± 60.37 | 0.0212* |
| SPRAYED | | | | |
| TMAO DI- | | | | |
| HYDRATE | | | | |
| SOLUTION | | | | |
| 1 G/L | 10 | WATER | 1371.4 ± 66.13 | 0.0073* |
| WATERED | | | | |
| TMAO DI- | | | | |
| HYDRATE | | | | |
| SOLUTION | | | | |
| — | 70 | CONTROL | 1109.3 ± 33.93 | — |
| — | 68 | 1 G/L TMAO DI- | 1216.1 ± 33.44 | 0.0265* |
| | | HYDRATE | | |

Table 15 shows that TMAO di-hydrate can be applied exogenously by spray and watering before the drought stress occurs increasing the plant survival rate and average dry weight in monocotyledonous plants, under extreme drought stress conditions. In the first three rows the initial treatments are compared, both 1 g/L TMAO di-hydrate spray (row 2) and 1 g/L TMAO di-hydrate irrigation treatments (row 3) significantly increase the mean dry weight per plant, under extreme drought conditions, after three cycles of wilt-watering, to 1205.4 mg and 1371.4 respectively when compared with water treated control plants in row 1 (1017.7 mg). Furthermore, similar results can be obtained when plants are only irrigated with 1 g/L TMAO di-hydrate (row 5: 1216.1 mg per plant) when compared with the same amount of limited irrigation with water without TMAO di-hydrate in row 4 (1109.3 mg).

### Example 24

### Corn Plants Treated with TMAO Di-Hydrate Recover Better from Drought Stress than Plants Irrigated with Water

TMAO di-hydrate applied exogenously which increases the endogenous content of TMAO as if the plants were overexpressing an FMO protein at least 4 times increases plant production in corn under limited water irrigation. Plants were irrigated with 30% of the water they normally require. ‘FAO700’ corn seeds were sown, grown and treated as described. Spray treatments with 1 g/L TMAO increased plant number of green leaves.

TABLE 16
Average number of green leaves and ANOVA analysis for TMAO
di-hydrate spray or seed treated corn plants under limited water
growing conditions

| | | | AVERAGE | |
|---|---|---|---|---|
| IRRIGATION | | | NUMBER OF | P- |
| REGIME | N | TREATMENT | GREEN LEAVES | VALUE |
| 100% | 30 | — | 11.03 ± 0.33 | — |
| WATER | | | | |
| 30% | 23 | — | 5.78 ± 0.38 | — |
| WATER | | | | |
| 30% | 53 | 1 g/L TMAO | 8.50 ± 0.25 | 0.0000 * |
| WATER | | SPRAY | | |
| 30% | 20 | 1 g/L TMAO | 8.50 ± 0.41 | 0.0001 * |
| WATER | | SEED | | |

Table 16 shows that TMAO can be applied exogenously by spray before the drought stress occurs, or by seed incubation, increasing the biomass production in the monocotyledonous plants, under limited drought stress conditions. In row 2 it is shown that 30% water irrigation significantly lowers the number of green leaves when compared with plants in row 1 under normal water irrigation. However, as shown in rows 3 and 4, spray treatment with 1 g/L of TMAO di-hydrate when applied exogenously every 4 weeks significantly restores the number of green leaves under limited water irrigation with a 47% increase in biomass production, shown in green leaf production over the untreated plants with a 30% irrigation.

### Example 25

### Corn Plants Treated with TMAO Recover Better from Drought Stress than Plants Irrigated with Water

TMAO di-hydrate applied exogenously increases plant production in corn which increases the endogenous content of TMAO as if the plants were overexpressing an FMO protein at least 4 times under limited water irrigation. ‘FAO700” corn seeds were sown, grown and treated as described above. As shown in Table 17, spray treatments with 1 g/L TMAO di-hydrate increased plant total chlorophyll content. After three months, leaf tissue samples of each plant were immersed for 18 hours in 80% ethanol. After this time, the absorbance of the suspension (OD₆₆₃) was determined as an indicator of chlorophyll concentration.

TABLE 17
Average chlorophyll content and ANOVA analysis for TMAO spray or
seed treated corn plants under limited water growing conditions

| | | | OD₆₆₃ | |
|---|---|---|---|---|
| IRRIGATION | | | ABSORVANCE | P- |
| REGIME | | TREATMENT | (CHLOROPHYLL a) | VALUE |
| 100% WATER | 0 | — | 0.9163 ± 0.052 | — |
| 30% WATER | 3 | — | 0.5194 ± 0.107 | — |
| 30% WATER | 3 | 1 g/L TMAO | 0.7278 ± 0.076 | 0.1214 |
| | | SPRAY | | |

Table 17 shows that TMAO di-hydrate can be applied exogenously by spray before the drought stress occurs, or by seed incubation, increasing the total chlorophyll content in corn plants, under limited drought stress conditions. In row 2 it is shown that 30% water irrigation significantly lowers total chlorophyll content when compared with plants in row 1 under normal water irrigation. However, as shown in rows 3 and 4, spray treatment with 1 g/L of TMAO di-hydrate when applied exogenously every 4 weeks significantly restores the chlorophyll content under limited water irrigation with an increase in biomass production between 40% and 72%, shown in chlorophyll content over the untreated plants with a 30% irrigation.

### Example 26

### Corn Plants Treated with TMAO Di-Hydrate Recover Better from Drought Stress than Plants Irrigated with Water

TMAO di-hydrate applied exogenously which increases the endogenous content of TMAO as if the plants were overexpressing an FMO protein at least 4 times increases plant production in corn under limited water irrigation. ‘FAO700’ corn seeds were sown, grown and treated as described above. Spray treatments with 1 g/L TMAO di-hydrate increased plant grain production.

TABLE 18
Average number of grains per cob and ANOVA analysis for TMAO
di-hydrate spray or seed treated corn plants under limited water
growing conditions

| | | | AVERAGE | |
|---|---|---|---|---|
| | | | NUMBER OF | |
| IRRIGATION | | | GRAINS PER | P- |
| REGIME | N | TREATMENT | COB | VALUE |
| 100% WATER | 30 | — | 533.95 ± 22.48 | — |
| 30% WATER | 23 | — | 429.13 ± 45.31 | — |
| 30% WATER | 53 | 1 g/L TMAO | 511.34 ± 19.70 | 0.0495 * |
| | | SPRAY | | |
| 30% WATER | 20 | 1 g/L TMAO | 542.89 ± 41.22 | 0.0757 |
| | | SEED | | |

Table 18 shows that TMAO di-hydrate can be applied exogenously by spray before the drought stress occurs, or by seed incubation, increasing the average number of grains per cob in corn plants, under limited water conditions. In row 2 it is shown that 30% water irrigation significantly lowers total number of grains per corn cob when compared with plants in row 1 under normal water irrigation. However, as shown in rows 3 and 4, spray treatment with 1 g/L of TMAO di-hydrate when applied exogenously every 4 weeks significantly restores the total number of grains per corn cob under limited water irrigation with an increase in the average number of grains per cob of between 19% and 27%. Of note, row 4 actually shows a 2% increase in the total number of grains per corn cob for corn plants under 30% water irrigation with a spray treatment of 1 g/L of TMAO di-hydrate when compared to corn plants with 100% water irrigation.

### Example 27

### Broccoli Plants Treated with TMAO Di-Hydrate in Irrigation Produce More than Plants Irrigated without TMAO

TMAO di-hydrate applied exogenously which increases the endogenous content of TMAO as if the plants were overexpressing an FMO protein at least 4 times increases plant production in broccoli. Parthenon broccoli seeds were sown, grown and treated as described above. Constant irrigation with 1 g/L TMAO di-hydrate increased plant inflorescence production.

TABLE 19
Average fresh weight in grams per inflorescence and ANOVA analysis
for TMAO di-hydrate constant irrigation broccoli plants under limited
water growing conditions

| | | | AVERAGE FRESH | |
|---|---|---|---|---|
| | | | WEIGHT | |
| IRRIGATION | | | (GRAMS) PER | |
| REGIME | N | TREATMENT | INFLORESCENCE | P-VALUE |
| 100% | 12 | — | 129.6 ± 16.2 | — |
| WATER | | | | |
| 100% | 15 | 1 g/L TMAO | 220.2 ± 16.6 | 0.0001* |
| WATER | | | | |

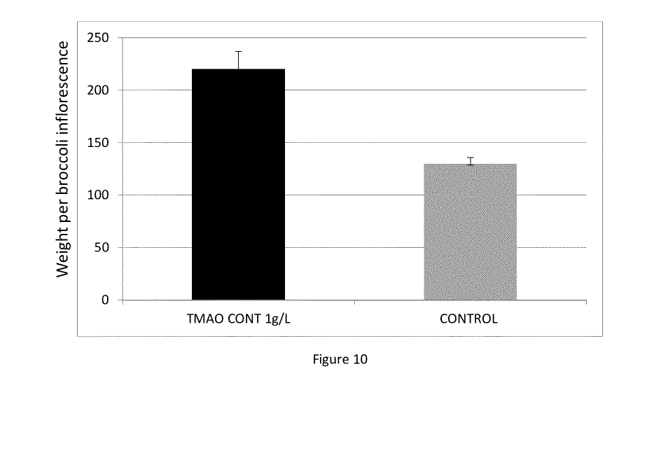

FIG. 10 is a bar graph of the data presented in Table 19. FIG. 10 and Table 19 show that TMAO di-hydrate can be applied exogenously by mixing it with the irrigation mixture even in the absence of stress, or by seed incubation, increasing the average broccoli inflorescence fresh weight. In row 2 it is shown that the constant irrigation with 1 g/L of TMAO di-hydrate significantly increases the broccoli inflorescence fresh weight by 70%.

### Example 28

### Pepper Plants Treated with TMAO Di-Hydrate in Irrigation or Spray Recover Better from Drought Stress than Plants Irrigated Only with Water and Fertilizer

TMAO di-hydrate applied exogenously which increases the endogenous content of TMAO as if the plants were overexpressing an FMO protein at least 4 times increases pepper production per plant and pepper fruit weight under limited water irrigation and under no stress. ‘Palermo’ pepper seeds were sown, grown and treated as described above. Constant irrigation with fertilization and spray treatments with 1 g/L TMAO or constant irrigation with fertilization mixed with 1 g/L TMAO treatment increased plant fruit production.

TABLE 20
Average fruit weight in grams production per pepper plant and
ANOVA analysis for TMAO di-hydrate spray or TMAO di-hydrate
in constant irrigation treated pepper plants under limited water
growing conditions

| | | | AVERAGE | |
|---|---|---|---|---|
| | | | FRUIT WEIGHT | |
| IRRIGATION | | | (GRAMS) PER | P- |
| REGIME | N | TREATMENT | PEPPER PLANT | VALUE |
| 100% | 14 | — | 481.2 ± 29.3 | — |
| WATER | | | | |
| 100% | 14 | 1 g/L TMAO | 567.4 ± 19.6 | 0.0216* |
| WATER | | IRRIGATION | | |
| 30% WATER | 28 | — | 361.9 ± 17.3 | — |
| 30% WATER | 28 | 1 g/L TMAO | 504.8 ± 46.4 | 0.001 * |
| | | SPRAY | | |
| 30% WATER | 28 | 1 g/L TMAO | 545.0 ± 36.4 | 0.001* |
| | | IRRIGATION | | |

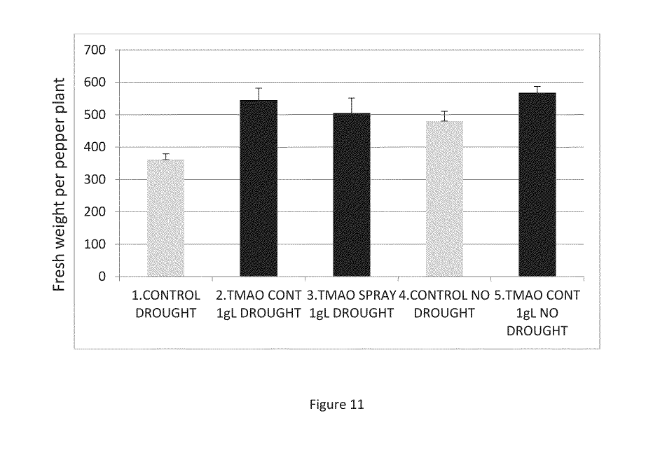

FIG. 11 is a bar graph of the data presented in Table 20. FIG. 11 and Table 20 show that TMAO di-hydrate can be applied exogenously by spray or added to the irrigation before the water stress occurs, increasing the average fruit weight production per pepper plant, under both limited water conditions and no stress conditions. In row 3 it is shown that a stress of 30% water irrigation significantly lowers total fruit weight production per pepper plant when compared with plants in row 1 under normal water irrigation. However, as shown in rows 4, spray treatment with 1 g/L of TMAO di-hydrate when applied exogenously every 4 weeks, and 5, irrigation treatment with 1 g/L of TMAO di-hydrate applied exogenously in every irrigation significantly restores the average fruit weight production per pepper plant under limited water irrigation with an increase in the average fruit weight production per pepper plant of between 39.5% and 50.6%. Of note, row 4 actually shows a 4.9% increase in the average fruit weight production per pepper plant for pepper plants under 30% water irrigation with a spray treatment of 1 g/L of TMAO di-hydrate and row 5 actually shows a 13.3% increase in the average fruit weight production per pepper plant for pepper plants under 30% water irrigation with an irrigation treatment with 1 g/L of TMAO di-hydrate applied exogenously in every irrigation when both are compared to pepper plants with no water stress or 100% irrigation in row 1. Furthermore as shown in row 2 the irrigation treatment with 1 g/L of TMAO di-hydrate applied exogenously in every irrigation, increases 17.9% in the average fruit weight production per pepper plant in the absence of stress at 100% water irrigation.

Table 21 shows that TMAO di-hydrate can be applied exogenously by spray or added to the irrigation before the water stress occurs, increasing the average weight per pepper fruit, under both limited water conditions and no stress conditions. In row 3 it is shown that a stress of 30% water irrigation significantly lowers average weight per pepper fruit when compared with plants in row 1 under normal water irrigation. However, as shown in rows 4, spray treatment with 1 g/L of TMAO di-hydrate when applied exogenously every 4 weeks, and 5, irrigation treatment with 1 g/L of TMAO di-hydrate applied exogenously in every irrigation significantly restores the average weight per pepper fruit under limited water irrigation with an increase in the average weight per pepper fruit t of between 24.9% and 40.7%. Of note, row 5 actually shows a 11.9% increase in the average weight per pepper fruit for pepper plants under 30% water irrigation with an irrigation treatment with 1 g/L of TMAO di-hydrate applied exogenously in every irrigation when are compared to pepper plants with no water stress or 100% irrigation in row 1.

TABLE 21
Average weight per pepper fruit and ANOVA analysis for TMAO
di-hydrate spray or TMAO di-hydrate in constant irrigation treated
pepper plants under limited water growing conditions

| | | | AVERAGE | |
|---|---|---|---|---|
| | | | WEIGHT | |
| IRRIGATION | | | (GRAMS) PER | |
| REGIME | N | TREATMENT | PEPPER FRUIT | P-VALUE |
| 100% | 155 | — | 33.7 ± 1.3 | — |
| WATER | | | | |
| 100% | 184 | 1 g/L TMAO | 35.7 ± 1.3 | 0.283* |
| WATER | | IRRIGATION | | |
| 30% WATER | 264 | — | 26.8 ± 0.8 | — |
| 30% WATER | 304 | 1 g/L TMAO | 33.5 ± 0.9 | 0.000 * |
| | | SPRAY | | |
| 30% WATER | 277 | 1 g/L TMAO | 37.7.0 ± 1.1 | 0.000* |
| | | IRRIGATION | | |

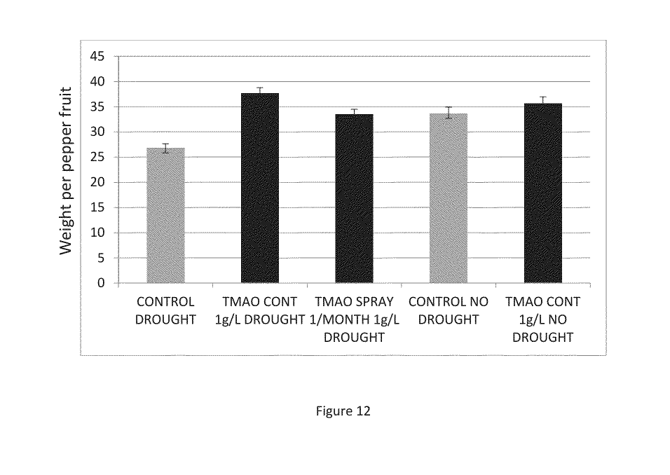

FIG. 12 is a bar graph of the data presented in Table 21. As shown in FIGS. 11 and 12 and Tables 20 and 21, TMAO di-hydrate can be applied exogenously by spray or added to the irrigation before the water stress occurs, increasing both the number of peppers per plant as well as the average weight per pepper fruit, under both limited water stress conditions and no stress conditions.

### Example 29

### Barley Seeds and Plants Treated with TMAO Di-Hydrate have an Increased Seed Production

TMAO di-hydrate applied exogenously which increases the endogenous content of TMAO as if the plants were overexpressing an FMO protein at least 4 times increases seed production in barley grown in the field without irrigation. ‘Hispanic” barley seeds were sown, grown and treated as described. Both, seed treatments (each Kg of seed was soaked in 1 liter of a 1 g/1 L TMAO di-hydrate solution, although smaller volumes of this solution are also effective) and a combination of seed and spray treatments with 1 g/L TMAO di-hydrate increased plant grain production. The field experienced 200 l/m² of rain water in total through the season.

TABLE 22
Average seed production in grams per square meter and ANOVA
analysis for TMAO seed or seed and spray treated barley plants grown
in the field without external irrigation

| | | Average number of | | |
|---|---|---|---|---|
| No. of | | Grams per square | | |
| samples | Treatment | meter | P-value | % Control |
| 8 | — | 190.63 ± 26.24 | — | — |
| 8 | 1 g TMAO/1 Kg | 225.98 ± 11.89 | 0.04615 * | 18 |
| | SEED | | | |
| 8 | 1 g TMAO/1 Kg | 256.36 ± 12.78 | 0.0438 * | 35 |
| | SEED + 1 g/L | | | |
| | TMAO spray | | | |

Table 22 shows that TMAO can be applied exogenously by spray before the drought stress occurs, or by seed incubation, increasing the seed production in barley plants grown in the open field without additional irrigation. Row one shows the number of samples (1 sqm/sample). In row 2 it is shown that seed treatment with 1 g of TMAO per 1 Kg of seeds significantly increases up to 18% the yield when compared with plants in row 1 without treatment. Furthermore, as shown in row 4, an additional spray treatment with 1 g/L of TMAO di-hydrate spray increases the total yield per square meter up to 35% when compared with the untreated control.

### Example 30

### Sunflower Seeds Treated with TMAO Produce Plants Having Increased Chlorophyll Content and Seed Production

TMAO di-hydrate applied exogenously which increases the endogenous content of TMAO as if the plants were overexpressing an FMO protein at least 4 times increases plant production in sunflower plants grown in the field without external irrigation. ‘Sambra” sunflower seeds were sown, grown and treated as described above. Seed treatment (1 g/l/Kg TMAO) increased plant chlorophyll content and seed production. Table 24 shows the chlorophyll content, weight of seeds and P-values for the ANOVA test. Both chlorophyll and weight differences between control and TMAO groups are statistically significant. Relative chlorophyll content values are obtained by optical absorbance in two different wavebands: 653 nm (chlorophyll) and 931 nm (Near Infra-Red).

TABLE 23
Effects of seed treatment with TMAO on plant fitness in sunflower
under natural stress conditions

| | | | | % GAIN/LOSS | |
|---|---|---|---|---|---|
| | | | AVERAGE | RESPECT TO THE | ANOVA |
| TRAIT | GROUP | N | VALUE | CONTROL | P-VALUE |
| CHLOROPHYLL | CONTROL | 100 | 16.28 ± 0.42 | 30% | 0.0000 |
| CONTENT | SEED | 100 | 21.17 ± 0.54 | | |
| (OD₆₆₃/OD₉₃₁ | TREATMENT | | | | |
| WEIGHT | CONTROL | 8 | 90.8 ± 9.0 | 77.7% | 0.0005 |
| (GRAMS) OF | SEED | 8 | 161.3 ± 13.1 | | |
| SEEDS FROM | TREATMENT | | | | |
| 1 PLANT | | | | | |

Table 23 shows that TMAO can be applied exogenously by seed treatment before the drought stress occurs, increasing the seed production in and oil bearing crop plants such as sunflower grown in the open field without additional irrigation. In column 5 it is shown that seed treatment with 1 g TMAO per 1 Kg seeds significantly increases up to 30% the chlorophyll content and the seed yield up to 77% when compared with control plants without treatment.

### Example 31

### TMAO Accumulates in Pepper and Barley after 1 Week Drought Stress

TMAO content in plants was determined by harvesting three leaves per treatment and freezing them in liquid nitrogen before the Nuclear Magnetic Resonance spectroscopy (NMR) determination. At least three independent plants were treated per experiment. TMAO content in plant extracts was quantified by NMR spectrometry using a Bruker Advance DRX 500 MHz spectrometer equipped with a 5 mm inverse triple resonance probe head. A known concentration of [3-(trimethylsilyl) propionic-2,2,3,3-d4 acid sod. salt, (TSP-d4)] was used as internal reference. All experiments were conducted at 298K and the data was acquired and processed using the same parameters. Spectra processing was performed on PC station using Topspin 2.0 software (Bruker).

‘Murano’ pepper and ‘Bomi’ barley seeds were sown and grown as described above. Control plants (six weeks old) were irrigated with 40 ml of water twice in the week, while “drought” treated plants were not irrigated. Leaves were harvested and TMAO was determined by NMR as described above. As shown in Table 24, TMAO levels increase almost three fold compared to the control in both pepper and barley after drought stress.

TABLE 24
TMAO accumulation after 1 week drought

| Crop | TMAO (μM) | SD | % Control |
|---|---|---|---|
| Pepper Control. | 446.68 | 215.86 | 100 |
| Pepper Drought 7 days | 1224.23 | 243.10 | 274 |
| Barley Control | 422.10 | 43.36 | 100 |
| Barley Drought 7 days | 1252.73 | 251.99 | 297 |

As shown in Table 24, in row 1, the control pepper shows 446.68 μM of TMAO, while in row 2 it is shown that 7 days of drought treatment increases TMAO levels in pepper 2.74 fold to 1224.23 μM. Similarly in row 3 control barley shows 422.10 μM of TMAO while in row 4 it is shown that 7 days of drought treatment increases TMAO levels in barley 2.97 fold to 1252.73 μM.

### Example 32

### TMAO Accumulates in Pepper and Barley when Applied Exogenously

‘Murano’ pepper seeds and ‘Bomi’ barley seeds were sown and grown as described above. Control plants (six weeks old) were sprayed with water and pepper treated plants were sprayed with 1 g/l of TMAO di-hydrate while barley plants were sprayed with 1 g/l of TMAO di-hydrate formulated with 0.1% of C8-C10 Alkylpolysaccharide. Leaves were harvested and TMAO was determined by NMR. The percentage of TMAO increase compared to untreated controls was determined for each time point.

TABLE 25
TMAO accumulation after TMAO di-hydrate spray treatments

| Crop | TMAO (μM) | SD | % Control |
|---|---|---|---|
| Pepper control | 331.8 | 78.3 | — |
| Pepper 1 day post spray | 1755.2 | 113.2 | 529 |
| Pepper 10 days post spray | 1237.6 | 138.4 | 373 |
| Pepper 20 days post spray | 948.9 | 166.7 | 286 |
| Pepper 30 days post spray | 449.2 | 251.99 | 135 |
| Pepper 40 days post spray | 709.4 | 152.9 | 213 |
| Barley control | 563.5 | 26.9 | — |
| Barley 1 day post spray | 4633.2 | 702.2 | 822 |

TMAO levels increase in pepper and barley with exogenous treatment of TMAO at 1 g/l to higher levels than drought treatment and furthermore, the TMAO levels are high up to 40 days post spray in pepper. As shown in Table 25, pepper and barley plants post TMAO di-hydrate spray exhibit between 1.1 and 9.9 fold greater level of endogenous TMAO compared to control plants that have not been treated with TMAO di-hydrate.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions, and sub-combinations as are within their true spirit and scope.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments for the purpose of streamlining the disclosure. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the disclosure.

The use of the terms “a,” “an,” and “the,” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

We claim:

1. A method of producing a transgenic photosynthetic organism or plant overexpressing an FMO protein, wherein the method comprises:
transforming a photosynthetic organism, plant, plant cell, or plant tissue with a sequence encoding a FMO protein operably linked to a promoter;
selecting for a photosynthetic organism, plant, plant cell, or plant tissue having said sequence stably integrated into said photosynthetic organism, plant, plant cell, or plant tissue genome, wherein said selecting comprises determining the level of expression of said FMO protein and selecting a photosynthetic organism having between 4 and 37 fold greater expression of said FMO protein compared to wild type; and
producing a transgenic photosynthetic organism or plant overexpressing an FMO protein.

2. The method of claim 1, wherein said selecting further comprising selecting for a photosynthetic organism, plant, plant cell, or plant tissue having two said sequences stably integrated into said photosynthetic organism, plant, plant cell, or plant tissue genome.

3. The method of claim 1 wherein the overexpression of said FMO protein is between 4.1 and 9.9 fold greater the level of expression compared to non-transformed plants and photosynthetic organisms.

4. The method of claim 1 wherein the overexpression of said FMO protein is between 10 and 16.9 fold greater the level of expression compared to non-transformed plants and photosynthetic organisms.

5. The method of claim 1 wherein the overexpression of said FMO protein is between 17 and 24.9 fold greater the level of expression compared to non-transformed plants and photosynthetic organisms.

6. The method of claim 1 wherein the overexpression of said FMO protein is between 25 and 36.9 fold greater the level of expression compared to non-transformed plants and photosynthetic organisms.

7. The method of claim 1, wherein the overexpression of said FMO protein catalyzes the oxidation of endogenous metabolites containing nucleophilic nitrogen.

8. The method of claim 1, wherein said FMO protein coding sequence comprises a nucleic acid molecule coding for a functionally equivalent variant of an FMO protein having at least 40% identity to the sequence as shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38 SEQ ID NO: 40, SEQ ID NO: 42 and SEQ ID NO: 43

9. The method of claim 8, wherein said FMO protein coding sequence comprises a nucleic acid molecule coding for a functionally equivalent variant of an FMO protein having between 90% and 100% identity to the sequence as shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38 SEQ ID NO: 40, SEQ ID NO: 42 and SEQ ID NO: 43.

10. The method of claim 1, wherein said FMO protein coding sequence comprises an amino acid molecule coding for a functionally equivalent variant of an FMO protein having at least 80% identity to the sequence as shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41 or SEQ ID NO: 44.

11. The method of claim 10, wherein said FMO protein coding sequence comprises a nucleic acid molecule coding for a functionally equivalent variant of an FMO protein having between 90% and 100% identity to the sequence as shown in SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38 SEQ ID NO: 40, SEQ ID NO: 42 and SEQ ID NO: 43.

12. The method of claim 1, wherein the promoter is a constitutive promoter.

13. The method of claim 1, wherein the promoter is a tissue specific promoter.

14. The method of claim 1, wherein the promoter is a stress inducible promoter.

15. The method of claim 14, wherein said stress inducible promoter is induced by drought stress.

16. The method of claim 1, further comprising a selectable marker operably linked to a promoter.

17. A transgenic plant produced by the method of claim 1, wherein said plant is drought tolerant.

18. A transgenic tissue culture of cells produced from the plant of claim 17, wherein the cells of the tissue culture are produced from a plant part chosen from leaves, pollen, embryos, cotyledons, hypocotyl, meristematic cells, roots, root tips, pistils, anthers, flowers, and stems, and wherein said tissue culture of cells overexpresses an FMO protein between 4 and 37 fold greater compared to non-transformed cells.

19. A transgenic plant regenerated from the tissue culture of claim 18.

20. A transgenic plant produced by the method of claim 1, wherein said plant has between 1.1 and 3.4 fold increase in trimethylamine N-oxide compared to wild-type.

21. A DNA construct comprising:
a promoter operably linked to a marker; and
a promoter operably linked to one or more FMO protein coding sequences, wherein said promoter operably linked to one or more FMO protein coding sequences is selected from the group consisting of 35S, Pro_{RD29A}, and Ubiquitin, and wherein said one or more FMO protein coding sequences has between 90% and 100% identity to the sequence as shown in SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, SEQ ID NO: 21, SEQ ID NO: 23, SEQ ID NO: 25, SEQ ID NO: 27, SEQ ID NO: 29, SEQ ID NO: 31, SEQ ID NO: 33, SEQ ID NO: 35, SEQ ID NO: 37, SEQ ID NO: 39, SEQ ID NO: 41 or SEQ ID NO: 44.

22. A drought tolerant transgenic plant having one or more DNA constructs stably integrated into said plants genome, wherein said DNA construct comprises an FMO protein coding sequence operably linked to a promoter, wherein said plant overexpresses said FMO protein between 4 and 37 fold greater than the level of FMO expression in non-transgenic plants, wherein said overexpression of said FMO protein catalyzes the oxidation of endogenous metabolites containing nucleophilic nitrogen, and wherein said transgenic plant has between 1.1 and 3.4 fold greater trimethylamine N-oxide.

23. The drought tolerant transgenic plant of claim 22, wherein said plant is a monocotyledonous or dicotyledonous plant.

24. The drought tolerant transgenic plant of claim 22, wherein said plant has an increased biomass under non-stressed conditions compared to wild-type plants.

25. The drought tolerant transgenic plant of claim 22, wherein said plant has an increased seed yield under non-stressed conditions compared to wild-type plants.
